SEQUENCE LISTING (38 sequences)

SEQ ID NO: 1 - protein, 194 aa, unidentified
  source 1..194: note = Humicola insolens
QLGAIENGLESGSANACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCTGTLIITPAHLSYTIEARGEAARFLRDRIRA

SEQ ID NO: 2 - protein, 298 aa, unidentified
  source 1..298: note = Caldimonas brevitalea
MPPDCVLPRRLAAAALLASATLVPLSAAAQTNPYQRGPDPTTRDLEDSRGPFRYASTNVRSPSGYGAGTIYYPTDVSGSVGAVAVVPGYLARQSSIRWWGPRLASHGFVVITLDTRSTSDQPASRSAQQMAALRQVVALSETRSSPIYGKVDPNRLAVMGWSMGGGGTLISARDNPSLKAAVPFAPWHNTANFSGVQVPTLVIACENDTVAPISRHASSFYNSFSSSLAKAYLEINNGSHTCANTGNSNQALIGKYGVAWIKRFVDNDTRYSPFLCGAPHQADLRSSRLSEYRESCPY

SEQ ID NO: 3 - protein, 290 aa, unidentified
  source 1..290: note = Ideonella sakaiensis strain NBRC 110686 / TISTR 2288 / 201-F6
MNFPRASRLMQAAVLGGLMAVSAAATAQTNPYARGPNPTAASLEASAGPFTVRSFTVSRPSGYGAGTVYYPTNAGGTVGAIAIVPGYTARQSSIKWWGPRLASHGFVVITIDTNSTLDQPSSRSSQQMAALRQVASLNGTSSSPIYGKVDTARMGVMGWSMGGGGSLISAANNPSLKAAAPQAPWDSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

SEQ ID NO: 4 - protein, 426 aa, unidentified
  source 1..426: note = Burkholderiales bacterium
MAVGSMLLSMAAQAQVVVFEETFSTGLGKFTAAGSVVTSSGAARLDGCYGCTDGSITSTAISTVDFTGLRLSFDRVTSGLDSGEAGIAEFSTNGSTYTAVESIRTASGRVTFNLPTSAENQSGLRLRFRINASLSSETYTVDNIRLEGTSGSGGGTTNPFEKGPDPTKTMLEASTGPFTYTTTTVSSTTASGYRQGTIYHPTNVTGPFAAVAVVPGYLASQSSINWWGPRLASHGFVVITIDTNSTSDQPPSRATQLMAALNQLKTFSNTSSHPIYRKVDPNRLGVMGWSMGGGGTLIAARDNPTLKAAIPFAPWNSSTNFSTVSVPTLIIACESDSTAPVNSHASPFYNSLPSTTKKAYLEMNNGSHSCANSGNSNAGLIGKYGVSWMKRFMDNDTRFSPYLCGAPHQADLSLTAIDEYRENCPY

SEQ ID NO: 5 - protein, 304 aa, unidentified
  source 1..304: note = Ideonella sakaiensis
MPFNKKSVLALCGAGALLFSMSALANNPAPTDPGDSGGGSAYQRGPDPSVSFLEADRGQYSVRSSRVSSLVSGFGGGTIYYPTGTTGTMGAVVVIPGFVSAESSIDWWGPKLASYGFVVMTIDTNTGFDQPPSRARQINNALDYLVSQNSRSSSPVRGMIDTNRLGVIGWSMGGGGTLRVASEGRIKAAIPLAPWDTTSYYASRSQAPTLIFACESDVIAPVLQHASPFYNSLPSSIDKAFVEINGGSHYCGNGGSIYNDVLSRFGVSWMKLHLDEDSRYKQFLCGPNHTSDSQISDYRGNCPY

SEQ ID NO: 6 - protein, 292 aa, unidentified
  source 1..292: note = Rhizobacter gummiphilus
MFGKLPFARASLAVGALLLSAAAVAQTNPYQRGPDPTVSSLEATRGPFSTSSFTVSRPSGYGAGTVYYPTNAGGKVGAIAVVPGYTARQSSINWWGPRLASHGFVVITIDTNSTLDQPSSRSSQQMAALRQVVSLAGTSSSPIYNKVDTARLGVMGWSMGGGGSLISAKNNPSLRAAAPQAPWAQESFSSVTVPTLIVSCENDSIAPNSSHSFPFYNQMTRNKKANLVINGGSHSCANSGNSDAGLIGKYGVAWMKRFMDDDTRYSKFLCGAEHQADLSKRAVEAYKENCPY

SEQ ID NO: 7 - protein, 293 aa, unidentified
  source 1..293: note = bacterium HR29
MQVVLGRVRSAGLLAALLALAAWALVWASPSAEAQSNPYQRGPNPTRSALTTDGPFSVATYSVSRLSVSGFGGGVIYYPTGTTLTFGGIAMSPGYTADASSLAWLGRRLASHGFVVIVINTNSRLDFPDSRASQLSAALNYLRTSSPSAVRARLDANRLAVAGHSMGGGATLRISEQIPTLKAGVPLTPWHTDKTFNTPVPQLIVGAEADTVAPVSQHAIPFYQNLPSTTPKVYVELDNATHFAPNSPNAAISVYTISWMKLWVDNDTRYRQFLCNVNDPALSDFRSNNRHCQ

SEQ ID NO: 8 - protein, 186 aa, Streptomyces scabiei
MAACTDIDVVSARGTFEPGTLGFIVGDPVYAALQKKVAGKSLSSYKVNYPADLSPTSAAQGNADLVNHVRSQAASCPNQRFVLVGYSQGANVVDNSIGISSAGAVVGSPIVATLPAALEPRVSAVLLFGNPIRAIGKSVTGTYQSRTIDFCAAGDPVCENGGGDVGAHLGYRANADAAAAFAATKI

SEQ ID NO: 9 - protein, 480 aa, Clostridium botulinum
  source 1..480: note = strain Hall / ATCC 3502 / NCTC 13319 / Type A
MKKNLNKIATIILLVFSMTLTNFSMIVRAAEPKAQGTQKVESSTTKKEVKDAEETIKIPTLEDIDNLIDSAEEVKSEEDINKMPPLKFPVEFPEVNTRSIIGGNNYPIVLVHGFMGFGRDELLGYKYWGGVVDLQEKLNASGHETYTATVGPVSSNWDRACELYAYIVGGTVDYGEAHAKKFKHNRYGRTYPGIYKNISNENKIHLIGHSMGGQTIRTLTQLLSEGSEEEINCGQENISPLFEGGKHWIHSVSTISTPNDGTTLSDLMPAKDLISYTFGVLGTITGKNKLFSSIYDLKLDQWGLKKQNGESQRDYIERVLDSNIWNSTKDIATYDLSTEGAQELNTWVKAQPDVYYFSWTTQATKESILTGHSVAQIGPMNPIFYPTANLMGRYSRNQKDLPIIDKKWFPNDGVVNCISQDGPKLGSNDVIEQYNGGVKIGQWNAMPRIINTDHMDIVGTFGNVKDWYMDYASFLSNLSR

SEQ ID NO: 10 - protein, 308 aa, unidentified
  source 1..308: note = uncultured bacterium
MPITARNTLASLLLASSALLLSGTAFAANPPGGDPDPGCQTDCNYQRGPDPTDAYLEAASGPYTVSTIRVSSLVPGFGGGTIHYPTNAGGGKMAGIVVIPGYLSFESSIEWWGPRLASHGFVVMTIDTNTIYDQPSQRRDQIEAALQYLVNQSNSSSSPISGMVDSSRLAAVGWSMGGGGTLQLAADGGIKAAIALAPWNSSINDFNRIQVPTLIFACQLDAIAPVALHASPFYNRIPNTTPKAFFEMTGGDHWCANGGNIYSALLGKYGVSWMKLHLDQDTRYAPFLCGPNHAAQTLISEYRGNCPY

SEQ ID NO: 11 - protein, 292 aa, Thermomonospora curvata
  source 1..292: note = strain ATCC 19995 / DSM 43183 / JCM 3096 / KCTC 9072 / NBRC 15933 / NCIMB 10081 / Henssen B9
MKRTLKRALSLLPAAALAASALVAASPAQAAANPYQRGPNPTEASITAARGPFNTAEITVSRLSVSGFGGGKIYYPTTTSEGTFGAIAISPGFTAYWSSLEWLGHRLASQGFVVIGIETNTTLDQPDQRGQQLLAALDYLTQRSAVRDRVDASRLAVAGHSMGGGGSLEAAKARTSLKAAIPLAPWNLDKTWPEVRTPTLIIGGELDAVAPVATHSIPFYNSLSNAPEKAYLELDNASHFFPNITNTQMAKYMIAWMKRFIDDDTRYTQFLCPPPSTGLLSDFSDARFTCPM

SEQ ID NO: 12 - protein, 289 aa, Thermomonospora curvata
  source 1..289: note = strain ATCC 19995 / DSM 43183 / JCM 3096 / KCTC 9072 / NBRC 15933 / NCIMB 10081 / Henssen B9
MSLRKSFGLLSATAALVAGLVAAPPAQAAANPYQRGPDPTESLLRAARGPFAVSEQSVSRLSVSGFGGGRIYYPTTTSQGTFGAIAISPGFTASWSSLAWLGPRLASHGFVVIGIETNTRLDQPDSRGRQLLAALDYLTQRSSVRNRVDASRLAVAGHSMGGGGTLEAAKSRTSLKAAIPIAPWNLDKTWPEVRTPTLIIGGELDSIAPVATHSIPFYNSLTNAREKAYLELNNASHFFPQFSNDTMAKFMISWMKRFIDDDTRYDQFLCPPPRAIGDISDYRDTCPHT

SEQ ID NO: 13 - protein, 296 aa, Thermobifida alba
MSVTTPRREASLLSRAVAVAAAAAATVALAAPAQAANPYERGPNPTESMLEARSGPFSVSEERASRLGADGFGGGTIYYPRENNTYGAIAISPGYTGTQSSIAWLGERIASHGFVVIAIDTNTTLDQPDSRARQLNAALDYMLTDASSSVRNRIDASRLAVMGHSMGGGGTLRLASQRPDLKAAIPLTPWHLNKSWRDITVPTLIIGADLDTIAPVSSHSEPFYNSIPSSTDKAYLELNNATHFAPNITNKTIGMYSVAWLKRFVDEDTRYTQFLCPGPRTGLLSDVDEYRSTCPF

SEQ ID NO: 14 - protein, 489 aa, Bacillus subtilis
MTHQIVTTQYGKVKGTTENGVHKWKGIPYAKPPVGQWRFKAPEPPEVWEDVLDATAYGPICPQPSDLLSLSYTELPRQSEDCLYVNVFAPDTPSQNLPVMVWIHGGAFYLGAGSEPLYDGSKLAAQGEVIVVTLNYRLGPFGFLHLSSFNEAYSDNLGLLDQAAALKWVRENISAFGGDPDNVTVFGESAGGMSIAALLAMPAAKGLFQKAIMESGASRTMTKEQAASTSAAFLQVLGINEGQLDKLHTVSAEDSLKAADQLRIAEKENIFQLFFQPALDPKTLPEEPEKAIAEGAASGIPLLIGTTRDEGYLFFTPDSDVHSQETLDAALEYLLGKPLAEKAADLYPRSLESQIHMMTDLLFWRPAVAYASAQSHYAPVWMYRFDWHPKKPPYNKAFHALELPFVFGNLDGLERMAKAEITDEVKQLSHTIQSAWITFAKTGNPSTEAVNWPAYHEETRETLILDSEITIENDPESEKRQKLFPSKGE

SEQ ID NO: 15 - protein, 262 aa, Thermobifida fusca
MANPYERGPNPTDALLEASSGPFSVSEENVSRLSASGFGGGTIYYPRENNTYGAVAISPGYTGTEASIAWLGGRIASHGFVVITIDTITTLDQPDSRAEQLNAALNHMINRASSTVRSRIDSSRLAVMGHSMGGGGTPRLASQRPDLKAAIPLTPWHLNKNRSSVTVPTLIIGADLDTIAPVATHAKPFYNSLPSSISKAYLELDGATHFAPNIPNKIIGKYSVAWLKRFVDNDTRYTQFLCPGPRDGLFGEVEEYCSTCPF

SEQ ID NO: 16 - protein, 262 aa, Thermobifida cellulosilytica
MANPYERGPNPTDALLEASSGPFSVSEENVSRLSASGFGGGTIYYPRENNTYGAVAISPGYTGTEASIAWLGERIASHGFVVITIDTITTLDQPDSRAEQLNAALNHMINRASSTVRSRIDSSRLAVMGHSMGGGGTLRLASQRPDLKAAIPLTPWHLNKNWSSVTVPTLIIGADLDTIAPVATHAKPFYNSLPSSISKAYLELDGATHFAPNIPNKIIGKYSVAWLKRFVDNDTRYTQFLCPGPRDGLFGEVEEYRSTCPF

SEQ ID NO: 17 - protein, 262 aa, Thermobifida cellulosilytica
MANPYERGPNPTDALLEARSGPFSVSEERASRFGADGFGGGTIYYPRENNTYGAVAISPGYTGTQASVAWLGERIASHGFVVITIDTNTTLDQPDSRARQLNAALDYMINDASSAVRSRIDSSRLAVMGHSMGGGGTLRLASQRPDLKAAIPLTPWHLNKNWSSVRVPTLIIGADLDTIAPVLTHARPFYNSLPTSISKAYLELDGATHFAPNIPNKIIGKYSVAWLKRFVDNDTRYTQFLCPGPRDGLFGEVEEYRSTCPF

SEQ ID NO: 18 - protein, 300 aa, Thermobifida alba
MSVTTPRRETSLLSRALRATAAAATAVVATVALAAPAQAANPYERGPNPTESMLEARSGPFSVSEERASRFGADGFGGGTIYYPRENNTYGAIAISPGYTGTQSSIAWLGERIASHGFVVIAIDTNTTLDQPDSRARQLNAALDYMLTDASSAVRNRIDASRLAVMGHSMGGGGTLRLASQRPDLKAAIPLTPWHLNKSWRDITVPTLIIGAEYDTIASVTLHSKPFYNSIPSPTDKAYLELDGASHFAPNITNKTIGMYSVAWLKRFVDEDTRYTQFLCPGPRTGLLSDVEEYRSTCPF

SEQ ID NO: 19 - protein, 228 aa, unidentified
  source 1..228: note = Thermothielavioides terrestris (strain ATCC 38088 / NRRL 8126) (Thielavia terrestris)
MKFLPILCAAGLAAAAPTQPAGEAAVEARQLFSDTANDLENGVSSNCPKVIFICARGSTETGNLGSSVCPEVANGLKNYYPNQLWVQGVGGAYTADLASNALPGGTSTAAMQEAANMFNLAQQKCPNASVAAGGYSQGTAVVAGGIQSLSAAAKDQIKGVVLFGYTQAQQNHDTIPNFPVDKTMIFCAQGDLVCNGTLIVTAAHFSYITNGDASTKGPAWLHEKIGDA

SEQ ID NO: 20 - protein, 293 aa, synthetic construct
  source 1..293: note = Unknown prokaryotic organism
MDGVLWRVRTAALMAALLALAAWALVWASPSVEAQSNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELDNASHFAPNSNNAAISVYTISWMKLWVDNDTRYRQFLCNVNDPALSDFRTNNRHCQ

SEQ ID NO: 21 - protein, 262 aa, Thermobifida halotolerans
MANPYERGPNPTNSSIEALRGPFRVDEERVSRLQARGFGGGTIYYPTDNNTFGAVAISPGYTGTQSSISWLGERLASHGFVVMTIDTNTTLDQPDSRASQLDAALDYMVEDSSYSVRNRIDSSRLAAMGHSMGGGGTLRLAERRPDLQAAIPLTPWHTDKTWGSVRVPTLIIGAENDTIASVRSHSEPFYNSLPGSLDKAYLELDGASHFAPNLSNTTIAKYSISWLKRFVDDDTRYTQFLCPGPSTGWGSDVEEYRSTCPF

SEQ ID NO: 22 - protein, 230 aa, unidentified
  source 1..230: note = Fusarium vanettenii (Neocosmospora pisi)
MKFFALTTLLAATASALPTSNPAQELEARQLGRTTRDDLINGNSASCRDVIFIYARGSTETGNLGTLGPSIASNLESAFGKDGVWIQGVGGAYRATLGDNALPRGTSSAAIREMLGLFQQANTKCPDATLIAGGYSQGAALAAASIEDLDSAIRDKIAGTVLFGYTKNLQNRGRIPNYPADRTKVFCNTGDLVCTGSLIVAAPHLAYGPDARGPAPEFLIEKVRAVRGSA

SEQ ID NO: 23 - protein, 319 aa, unidentified
  source 1..319: note = Moraxella sp. (strain TA144)
MFIMIKKSELAKAIIVTGALVFSIPTLAEVTLSETTVSSIKSEATVSSTKKALPATPSDCIADSKITAVALSDTRDNGPFSIRTKRISRQSAKGFGGGTIHYPTNASGCGLLGAIAVVPGYVSYENSIKWWGPRLASWGFVVITINTNSIYDDPDSRAAQLNAALDNMIADDTVGSMIDPKRLGAIGWSMGGGGALKLATERSTVRAIMPLAPYHDKSYGEVKTPTLVIACEDDRIAETKKYANAFYKNAIGPKMKVEVNNGSHFCPSYRFNEILLSKPGIAWMQRYINNDTRFDKFLCANENYSKSPRISAYDYKDCP

SEQ ID NO: 24 - protein, 576 aa, unidentified
  source 1..576: note = Melanocarpus albomyces
MKGIAALVTAALLGRAVAAPPEPPTNVVEKRAAPTVEISTGTIVGTTRLATEAFNGIPFALPPVGQLRLKPPVRLNSSLGVFDASGIAPACPQFLADSDSNEFLAQVINTVTGLPFFQKALKISEDCLTINVIRPKGTKAGDKLPVLFWIYGGGFELGWSSMYDGGPLVTNAIGFGKPFIFVAVNYRVAGFGFMPGKEILADGAANLGHLDQRMGLEWVADNIAAFGGDPDKVTIWGESAGAISVLNQMALFDGDHTYKGKPLFRGAIMNSGSIVPADPVDCPKGQEIYDQVVAKAGCAGASDTLACLRELPYEKFLDAANSVPAILSYNSVALSYLPRPDGKVLTKSPDVLIKEGKYAAVPMIIGDQEDEGTLFSLFQPNITNSEKLVTYLKDLFFHGASREQLEGLVDRYPARISAGSPFRTGLLNEIYPGFKRLAAILGDLVFTLTRRVFLEDATRVNPTVPAWSYLASYDYGTPILGTFHGSDLLQVFFGILPNYASRSIQSYYANFVYNLDPNDASGGTSGKSKVAEEWPRWTADDRTLIQFFANRNGYLKDDFRSEAAEYIGAHVDYLHI

SEQ ID NO: 25 - protein, 301 aa, Thermobifida fusca
MAVMTPRRERSSLLSRALRFTAAAATALVTAVSLAAPAHAANPYERGPNPTDALLEARSGPFSVSEERASRFGADGFGGGTIYYPRENNTYGAVAISPGYTGTQASVAWLGKRIASHGFVVITIDTNTTLDQPDSRARQLNAALDYMINDASSAVRSRIDSSRLAVMGHSMGGGGSLRLASQRPDLKAAIPLTPWHLNKNWSSVRVPTLIIGADLDTIAPVLTHARPFYNSLPTSISKAYLELDGATHFAPNIPNKIIGKYSVAWLKRFVDNDTRYTQFLCPGPRDGLFGEVEEYRSTCPF

SEQ ID NO: 26 - protein, 310 aa, Oleispira antarctica
  source 1..310: note = Oleispira antarctica RB-8
MNKSILKKLSFGTSVLLVSMNALSWTPSPTPNPDPIPDPTPCQDDCDFTRGPNPTPSSLEASTGPYSVATRSVASSVSGFGGGTLHYPTNTTGTMGAIAVVPGFLLQESSIDFWGPKLASHGFVVITISANSGFDQPASRATQLGRALDYVINQSNGSNSPISGMVDTTRLGVVGWSMGGGGALQLASGDRLSAAIPIAPWNQGGNRFDQIETPTLVIACENDVVASVNSHASPFYNRIPSTTDKAYLEINGGSHFCANDGGSIGGLLGKYGVSWMKRFIDNDLRYDAFLCGPDHAANRSVSEYRDTCNY

SEQ ID NO: 27 - protein, 304 aa, Saccharomonospora viridis
MRIRRQAGTGARASMARAIGVMTTALAVLVGAVGGVAGAEVSTAQDNPYERGPDPTEDSIEAIRGPFSVATERVSSFASGFGGGTIYYPRETDEGTFGAVAVAPGFTASQGSMSWYGERVASQGFIVFTIDTNTRLDQPGQRGRQLLAALDYLVERSDRKVRERLDPNRLAVMGHSMGGGGSLEATVMRPSLKASIPLTPWNLDKTWGQVQVPTFIIGAELDTIASVRTHAKPFYESLPSSLPKAYMELDGATHFAPNIPNTTIAKYVISWLKRFVDEDTRYSQFLCPNPTDRAIEEYRSTCPY

SEQ ID NO: 28 - protein, 230 aa, unidentified
  source 1..230: note = Fusarium oxysporum f. sp. raphani 54005
MKFSIISTLLAATASALPAGQDAAALEARQLGGSITRNDLANGNSGSCPGVIFIYARGSTESGNLGTLGPRVASKLEAKYGKNGVWIQGVGGAYRATLGDNALPRGTSSAAIREMLGHFSDANQKCPDAVLIAGGYSQGAALAAASVTDVDAGIREKIAGVVLFGYTKNLQNRGKIPSYPEDRTKVFCNTGDLVCTGSLIVAAPHLAYQSAASGAAPEFLIQKADAAGAA

SEQ ID NO: 29 - protein, 302 aa, unidentified
  source 1..302: note = Halopseudomonas bauzanensis
MINKNLSQSLLAMMAAGALLLSSSAFAVNPPTDGPTDPDQAYERGPDPSVAFLEAPTGPHSVRTSRVSGLVSGFGGGTIHYPTGTTGTMAAIVVIPGFVSAESSIEWWGPKLASHGFVVMTIDTNTGFDQPPSRARQINNALDYLVSQNTSRTSPVNGMIDTERLGVIGWSMGGGGTLRVASEGRIKAAIPLAPWDTTRFRGVQAPTLIFACESDLIAPVRSHASPFYNQLPDDIDKAYVEINNGSHYCANGGGLNNDVLSRFGVSWMKRFLDNDTRYSQFLCGPNHESDRNISEYRGNCPY

SEQ ID NO: 30 - protein, 302 aa, unidentified
  source 1..302: note = Pseudomonas saudimassiliensis
MINKKLSQTLLSMAAAGALMFSASVFATNPPTDEPTNPGQSYERGPDPTVAFLEASSGPYSVRTSRVSGLVSGFGGGTIHYPTGTTGTMAAIVVIPGFVSAESSIDWWGPKLASHGFVVMTIDTNTGFDQPPSRARQINNALDYLVDQNSSRTSPVNGMIDTERLGVIGWSMGGGGTLRVASEGRIKAAIPLAPWDTTRFSGVQAPTLIFACESDLIAPVRSHASPFYNQLPNDIDKAYVEINNGSHYCANGGGLNNDVLSRFGVSWMKRFLDNDTRYSQFLCGPRHESDRKISEYRGNCPY

SEQ ID NO: 31 - protein, 298 aa, Vibrio gazogenes
MMNVLTKCKLALGIIAIFFSLPSFAVPCSDCSNGFERGQVPRVDQLESSRGPYSVKTINVSRLARGFGGGTIHYSTESGGQQGIIAVVPGYVSLEGSIKWWGPRLASWGFTVITIDTNTIYDQPDSRASQLSAAIDYVIDKGNDRSSPIYGLVDPNRVGVIGWSMGGGGSLKLATDRKIDAVIPQAPWYLGLSRFSSITSPTMIIACQADVVAPVSVHASRFYNQIPGTTPKAYFEIALGSHFCANTGYPSEDILGRNGVAWMKRFIDKDERYTQFLCGQNFDSSLRVSEYRDNCSYY

SEQ ID NO: 32 - protein, 315 aa, unidentified
  source 1..315: note = Deinococcus maricopensis (strain DSM 21211 / LMG 22137 / NRRL B-23946 / LB-34)
MSNGYSACPAPYDHRGMRKHASLLAALPVLLAACGTTTSTLTTPAVTPQTLSGAATNPYERGPAPTTALLEAARGPYATASTTVPRSSVSDFGGATIYYPTSTADGTFGGVAISPGYTGTQASVAWLGPRLASHGFVVIVIDTLSRYDYPSSRGDQLRAALRYLTTSSAVRTRVDATRLAVMGHSMGGGGALEAAKDNPALKAAIPLTPWNTDKSWPELTTPTLIFGAQNDSVAPVSSHAIPFYTSLASTLPKAYLELRGASHGAPTSTNTTIAKYAVAWLKRFEDADRRYDPFLCPTPAVSTLLSDARSTCPFN

SEQ ID NO: 33 - protein, 342 aa, unidentified
  source 1..342: note = Pseudozyma antarctica (Yeast) (Candida antarctica)
MKLLSLTGVAGVLATCVAATPLVKRLPSGSDPAFSQPKSVLDAGLTCQGASPSSVSKPILLVPGTGTTGPQSFDSNWIPLSTQLGYTPCWISPPPFMLNDTQVNTEYMVNAITALYAGSGNNKLPVLTWSQGGLVAQWGLTFFPSIRSKVDRLMAFAPDYKGTVLAGPLDALAVSAPSVWQQTTGSALTTALRNAGGLTQIVPTTNLYSATDEIVQPQVSNSPLDSSYLFNGKNVQAQAVCGPLFVIDHAGSLTSQFSYVVGRSALRSTTGQARSADYGITDCNPLPANDLTPEQKVAAAALLAPAAAAIVAGPKQNCEPDLMPYARPFAVGKRTCSGIVTP

SEQ ID NO: 34 - protein, 319 aa, Thermobifida fusca
  source 1..319: note = Thermobifida fusca (strain YX)
MPPHAARPGPAQNRRGRAMAVITPRRERSSLLSRALRFTAAAATALVTAVSLAAPAHAANPYERGPNPTDALLEARSGPFSVSEERASRFGADGFGGGTIYYPRENNTYGAVAISPGYTGTQASVAWLGERIASHGFVVITIDTNTTLDQPDSRARQLNAALDYMINDASSAVRSRIDSSRLAVMGHSMGGGGTLRLASQRPDLKAAIPLTPWHLNKNWSSVRVPTLIIGADLDTIAPVLTHARPFYNSLPTSISKAYLELDGATHFAPNIPNKIIGKYSVAWLKRFVDNDTRYTQFLCPGPRDGLFGEVEEYRSTCPF

SEQ ID NO: 35 - protein, 304 aa, Acidovorax delafieldii
  source 1..304: note = Acidovorax delafieldii (Pseudomonas delafieldii)
MHLPRSRWDIPFKEETTMTHHFSVRALLAAGALLASAAVSAQTNPYERGPAPTTSSLEASRGPFSYQSFTVSRPSGYRAGTVYYPTNAGGPVGAIAIVPGFTARQSSINWWGPRLASHGFVVITIDTNSTLDQPDSRSRQQMAALSQVATLSRTSSSPIYNKVDTSRLGVMGWSMGGGGSLISARNNPSIKAAAPQAPWSASKNFSSLTVPTLIIACENDTIAPVNQHADTFYDSMSRNPREFLEINNGSHSCANSGNSNQALLGKKGVAWMKRFMDNDRRYTSFACSNPNSYNVSDFRVAACN

SEQ ID NO: 36 - protein, 201 aa, Pseudomonas pseudoalcaligenes
  source 1..201: note = Pseudomonas pseudoalcaligenes (strain CECT 5344)
MRAWWLSGALALMFWAQGAVAGTLLVVGDSISAAFGLDSRQGWVALLEKRLSEEGFEHSVVNASISGDTSAGGAARLSALLAEHKPELVIIELGGNDGLRGQPPAQLQQNLASMVEQSQQAGAKVLLLGMKLPPNYGVRYTTAFAQVFTDLAEQKQVSLVPFFLEGVGGVPGMMQADGIHPAEAAQEILLDNVWPTLKPML

SEQ ID NO: 37 - protein, 259 aa, unidentified
  source 1..259: note = Compost metagenome, hydrolase gene, clone 3
MENPYERGPDPTESSIEAVRGPFAVAQTTVSRLQADGFGGGTIYYPTDTSQGTFGAVAISPGFTAGQESIAWLGPRIASQGFVVITIDTITRLDQPDSRGRQLQAALDHLRTNSVVRNRIDPNRMAVMGHSMGGGGALSAAANNTSLEAAIPLQGWHTRKNWSSVRTPTLVVGAQLDTIAPVSSHSEAFYNSLPSDLDKAYMELRGASHFVSNTPDTTTAKYSIAWLKRFVDNDLRYEQFLCPAPDDFAISEYRATCPF

SEQ ID NO: 38 - protein, 259 aa, unidentified
MANPYERGPDPTESSIEAVRGPFAVAQTTVSRLQADGFGGGTIYYPTDTSQGTFGAVAISPGFTAGQESIAWLGPRIASQGFVVITIDTITRLDQPDSRGRQLQAALDHLRTNSVVRNRIDPNRMAVMGHSMGGGGALSAAANNTSLEAAIPLQGWHTRKNWSSVRTPTLVVGAQLDTIAPVSSHSEAFYNSLPSDLDKAYMELRGASHLVSNTPDTTTAKYSIAWLKRFVDDDLRYEQFLCPAPDDFAISEYRSTCPF

(19) United States
(12) Patent Application Publication
(10) Pub. No.: US 2024/0352064 A1
(43) Pub. Date: Oct. 24, 2024

(54) METHODS AND COMPOSITIONS FOR THE SEPARATION OF PARTICLES FROM A FLUID

(71) Applicant: Protein Evolution Inc., Guilford, CT (US)

(72) Inventors: Brian Mansaku, Amston, CT (US); Benjamin Gibbs, North Haven, CT (US)

(73) Assignee: Protein Evolution Inc., Guilford, CT (US)

(21) Appl. No.: 18/610,932

(22) Filed: Mar. 20, 2024

Related U.S. Application Data
(60) Provisional application No. 63/491,520, filed on Mar. 21, 2023.

(51) Int. Cl. C07K 1/36; C07K 1/30; C07K 1/34

(52) U.S. Cl. CPC C07K 1/36; C07K 1/30; C07K 1/34

(57) ABSTRACT

Certain aspects of this disclosure are generally related to methods for separating a plurality of particles from a fluid. In some embodiments, the fluid comprises one or more polypeptides and a plurality of particles. In some embodiments, the method comprises at least partially denaturing a polypeptide in the fluid to form a denatured polypeptide. In some embodiments, the method comprises separating a mixture of the denatured polypeptide and at least 10% of a plurality of particles from the fluid. Without wishing to be bound by any particular theory, the denaturation of the polypeptide in the fluid may allow for some of the plurality of particles to associate with the denatured polypeptide, and/or agglomerate with the denatured polypeptide. The mixture comprising the denatured polypeptide and the plurality of particles may settle (e.g., precipitate) in the fluid and may therefore be separated from the fluid.

### RELATED APPLICATIONS

This application claims priority under 35 U.S.C. § 119 (e) to U.S. Provisional Patent Application No. 63/491,520, filed Mar. 21, 2023, and entitled “ORGANIC FLOCCULATION,” which is incorporated herein by reference in its entirety for all purposes.

### REFERENCE TO AN ELECTRONIC SEQUENCE LISTING

The contents of the electronic sequence listing (P118370018US01-SEQ-TJO.xml; Size: 50,189 bytes; and Date of Creation: Mar. 18, 2024) is herein incorporated by reference in its entirety.

### TECHNICAL FIELD

Methods and compositions related to the separation of particles from a fluid are generally described.

### BACKGROUND

The widespread use of plastics, such as polyethylene terephthalate (PET), makes the disposal of such materials particularly challenging. Often, large quantities of plastics and/or post-consumer/industrial polymeric material (PC/IPM) are disposed of in landfills that contaminate and/or pollute the environment. Microplastics are especially difficult to remove from water sources due to relatively small particles size. While the enzymatic degradation of polymeric materials has shown to be a promising solution for the disposal, repurposing, and/or recycling of polymeric materials, it is generally challenging to implement on commercial scales. Accordingly, scalable technologies to recycle materials and/or remove contaminants are needed.

### SUMMARY

Methods and compositions related to the separation of particles from a fluid are generally described. The subject matter of the present disclosure involves, in some cases, interrelated products, alternative solutions to a particular problem, and/or a plurality of different uses of one or more systems and/or articles.

One aspect is generally directed to a method for separating a plurality of particles from a fluid. In some embodiments, the method comprises, in a fluid comprising one or more polypeptides and a plurality of plastic particles, at least partially denaturing a polypeptide in the fluid; and separating a mixture comprising denatured polypeptide and at least 10% of the plastic particles from the fluid. In some embodiments, the method comprises separating a mixture comprising a plurality of plastic particles and one or more denatured polypeptides, from a supernatant comprising a fluid, wherein: the concentration of at least one of the polypeptides in the supernatant is less than or equal to 1×10⁻³ M.

Another aspect is generally directed to a composition related to the separation of a plurality of particles from a fluid. In some embodiments, the composition comprises one or more denatured polypeptides; and a plurality of plastic particles, having an average maximum dimension less than or equal to 2 mm, wherein the composition is the product of at least partial isolation from a solution or suspension comprising the plastic particles and the polypeptide. In some embodiments, the composition comprises a plurality of plastic particles, having an average maximum dimension less than or equal to 2 mm; and a polypeptide associated with the plurality of plastic particles, wherein at least 50% of the polypeptide is denatured. In some embodiments, the composition comprises a plurality of plastic particles, having an average maximum dimension less than or equal to 2 mm; and a denatured polypeptide associated with the plurality of plastic particles, wherein: the plastic particles, in the absence of the denatured polypeptide, are in suspension in a fluid, and the particles agglomerate and settle in the fluid in the presence of the denatured polypeptide, and the composition is the product of at least partial isolation, of the particles and polypeptide, from the fluid.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A is a cross-sectional schematic diagram of a fluid comprising a plurality of particles and a polypeptide, according to some embodiments.

FIG. 1B is a cross-sectional schematic diagram of an agglomerate separated from a fluid, according to some embodiments.

FIG. 2A is a cross-sectional schematic diagram of a fluid comprising a first polypeptide, a second polypeptide, and a plurality of particles, according to some embodiments.

FIG. 2B is a cross-sectional schematic diagram of an agglomerate separated from a fluid comprising a second polypeptide suspended in the fluid, according to some embodiments.

FIG. 3 depicts a particle size distribution (PSD) of PET particles prior to exposure to a polymer-degrading enzyme, according to some embodiments.

FIG. 4 depicts a particle size distribution (PSD) of PET particles after exposure to a polymer-degrading enzyme, according to some embodiments.

FIG. 5 depicts the OD600 of fluids separated from the plurality of particles via centrifugation, according to some embodiments.

FIG. 6A depicts an image of a vessel comprising PET particles suspended in a fluid, according to some embodiments.

FIG. 6B depicts the dosage of NaOH into a fluid over time, according to some embodiments.

FIG. 7 depicts the size distribution of particles within a supernatant, according to some embodiments.

FIG. 8 depicts an image showing limited separation between a plurality of particles and a fluid via centrifugation, according to some embodiments.

FIG. 9 depicts the addition of a flocculation (coagulant) agent into a fluid, according to some embodiments.

FIG. 10 depicts an image showing various examples of flocculation agents, according to some embodiments.

FIG. 11 depicts various filtration steps that may facilitate the separation of a plurality of particles from a fluid, according to some embodiments.

FIG. 12 is an image showing the isolation of a plurality of particles and a denatured polypeptide after exposure to a relatively high pH and a relatively high temperature, according to some embodiments.

FIG. 13 is an image showing the separation of a plurality of particles, associated with a denatured polypeptide, from the fluid, according to some embodiments.

FIG. 14 depicts the relative particle concentration in a fluid prior to separation, and after separation induced by exposing a polypeptide to relatively high pH and a relatively high temperature, according to some embodiments.

FIG. 15 is an exemplary determination of melting temperatures for the HiC 51032 enzyme, according to some embodiments.

FIG. 16 is a flow chart of a typical process and process implementing methods described herein, according to some embodiments.

### DETAILED DESCRIPTION

This disclosure generally relates to methods for separating a plurality of particles, including but not limited to plastic particles, from a fluid, and related compositions. The method involves exposing the particles to one or more polypeptides, then separating a combination of the particles and one or more polypeptides (optionally with some fluid included) from the bulk of the fluid. In some embodiments, the method involves at least partially denaturing polypeptide molecules (some but not necessarily all of those in the fluid), then separating a mixture of denatured polypeptide and at least some of the particles from the fluid (e.g., 10% of the particles, or more). Without wishing to be bound by any particular theory, the denaturation of polypeptide in the fluid may allow for some of the plurality of particles to associate with the denatured polypeptide, and/or agglomerate with the denatured polypeptide, for example by “like” interactions between accessible portions (e.g., hydrophobic portions) of denatured polypeptide and hydrophobic particles, or other “like/like” interactions which typically involved hydrogen bonding, van der Waals interactions, weak interactions, or the like. The mixture comprising the denatured polypeptide and the plurality of particles may settle (e.g., precipitate) in the fluid and may therefore be separated from the fluid, and/or can be removed by filtration, centrifugation, adsorption, or any technique which those of ordinary skill in the art, with their knowledge and this disclosure, can implement.

“Separate,” “separation,” and related terms, in the context of this disclosure, means moving a generally solid combination, for example particles and denatured polypeptide, from the bulk of a fluid. In such a case, some of the fluid may carry along with the generally solid mixture and, at a molecular level, the solid mixture remains in association with some fluid but at a greater concentration than had been the case prior to separation, and what is left behind is the bulk fluid with lower (in most cases much lower) concentration of the particles then had been the case prior to the separation. In some embodiments, nearly all or virtually all of the fluid is separated from the particles, in the disclosed method.

This disclosure generally relates to compositions as well. The compositions involve one or more denatured polypeptides and, in some cases, a plurality of particles (e.g., plastic particles). In certain instances, the composition may be a product of at least partial isolation, such as via filtration and/or centrifugation, from a solution or suspension (e.g., a fluid) comprising the plurality of plastic particles and the polypeptide. Some of the polypeptide may be at least partially denatured. The particles, in some cases, agglomerate and settle in the fluid in the presence of the denatured polypeptide and may be isolated (e.g., via filtration) to form the product. In some embodiments, the plastic particles, in the absence of the denatured polypeptide, are in suspension in a fluid.

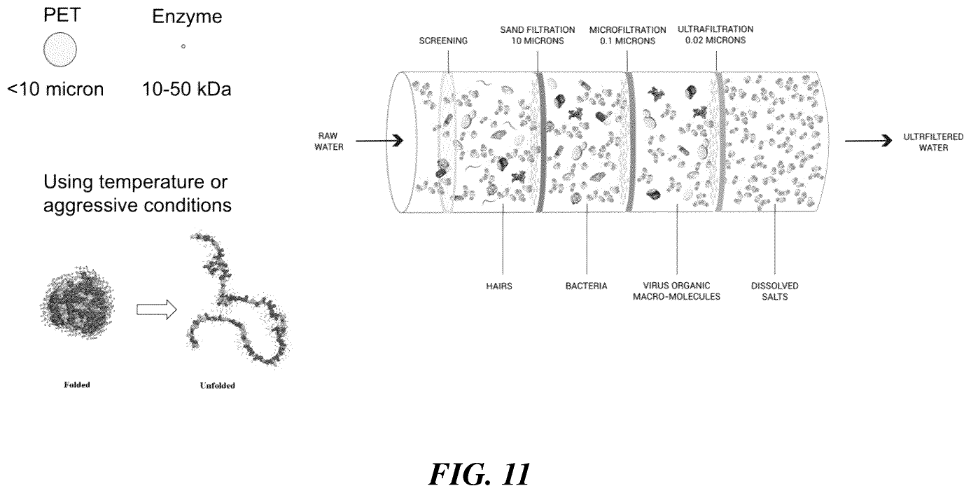

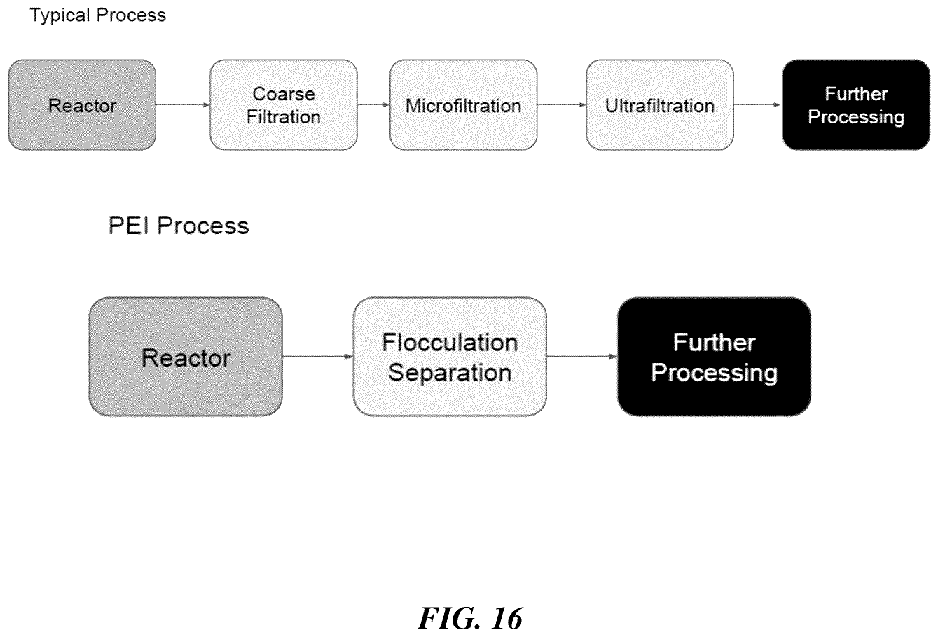

The separation of suspended particles (e.g., plastic particles) in a fluid is generally a difficult challenge to overcome in many industries. For example, the enzymatic degradation of post-consumer/industrial polymeric materials (e.g., PC/IPM) often necessitates the exposure of a plurality of particles to a polymer-degrading enzyme in a fluid. As the polymer-degrading enzyme degrades (e.g., depolymerizes) the plurality of particles, the average maximum dimensions of the particles may decrease allowing them to be suspended in the fluid. To separate the fluid, which may comprise commercially valuable degradation products, from the particles, filtration and centrifugation methods are typically implemented. However, these methods generally have low throughput and/or are not feasible at commercial scales. Particles (e.g., plastic particles) having a relatively small maximum dimension are particularly difficult to separate from a fluid. These relatively small particles have a tendency to clog filters and/or otherwise inhibit filtration of the fluid, and while the use of several filtration steps may implemented to promote filtration (FIG. 11 and FIG. 16), the throughput of the resulting filtration process is low and may not be suitable at commercial scales. These relatively small particles may be separated from the fluid via centrifugation using relatively high speeds (e.g., 12,000 rpm), but such centrifugation conditions are not feasible using commercially available centrifuges at large scales.

The presence of microplastics in fluids further demonstrate the need to separate particles from fluids. Microplastics are a form of plastic debris that is often found in the environment, include but not limited to water sources, and are typically difficult to remove due to their relatively small particle size. Microplastics, having a maximum dimension of less than or equal to 5 mm and greater than or equal to 1 nm, may be suspended in fluids that are destined for consumption and/or use in agricultural applications and pose significant health risks when consumed directly and/or indirectly. However, typical filtration and/or centrifugation process are may not effectively remove microplastics from fluids. Accordingly, there is a need for improved methods to separate a plurality of particles from a fluid.

As noted above, certain embodiments are related to methods for separating a plurality of particles (e.g., plastic particles) from a fluid. Without wishing to be bound by any particular theory, it was observed, in a fluid comprising the plurality of particles and one or more polypeptides, that denaturing one or more polypeptides may expose hydrophobic portions of the denatured polypeptide and allow the denatured polypeptides to associate with some of the plurality of particles suspended in the fluid. Upon association, a mixture comprising the particles associated with the denatured polypeptide may advantageously agglomerate and settle in the fluid thereby separating the particles from the fluid. Once separated, filtration methods, in some embodiments, may be implemented to isolate a mixture from the fluid. Surprisingly, the mixture may be separated and isolated from the fluid with relatively high throughput, and allow the fluid (e.g., supernatant) to have a relatively low concentration of particles and/or a relatively low concentration of the one or more polypeptides.

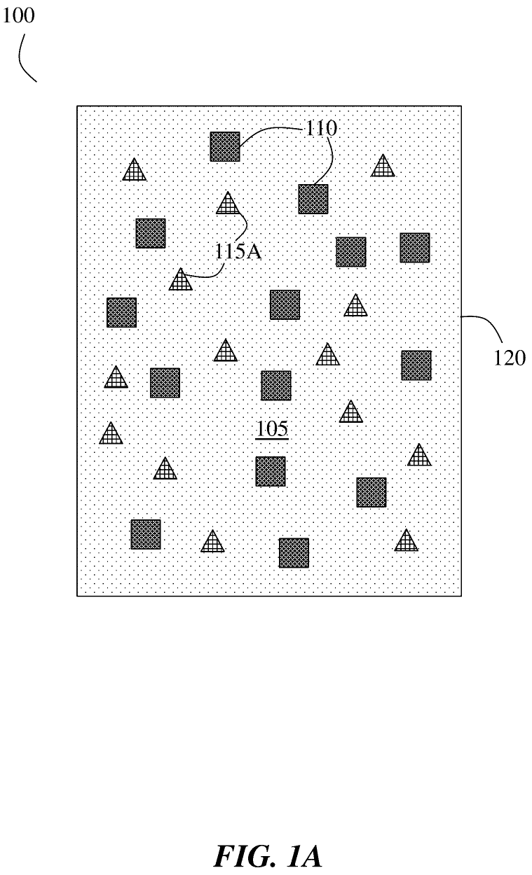

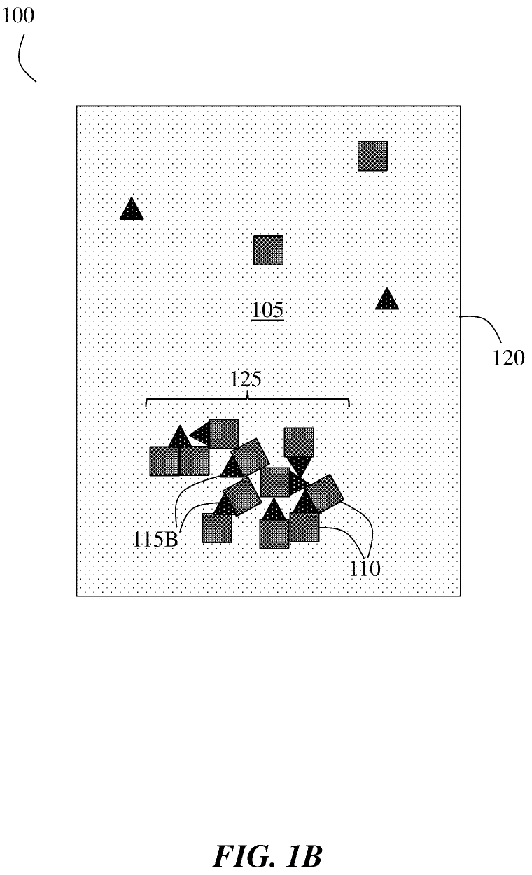

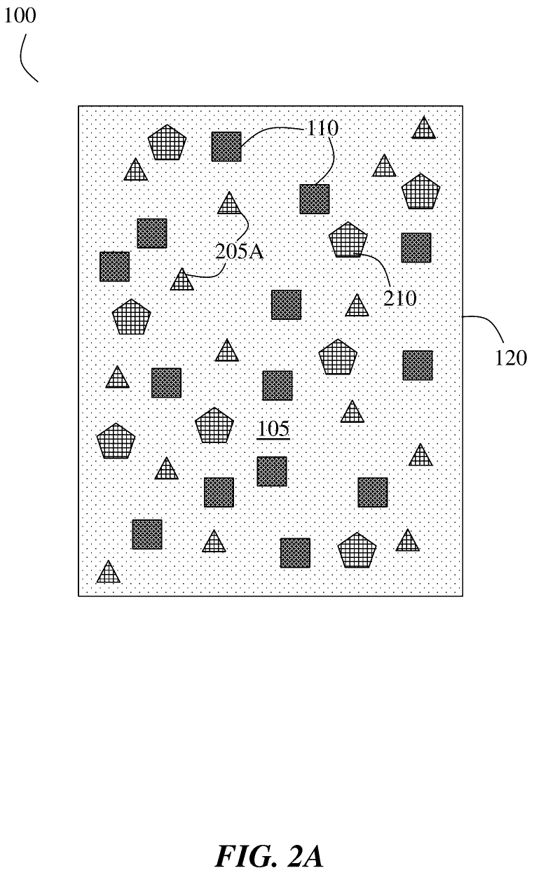

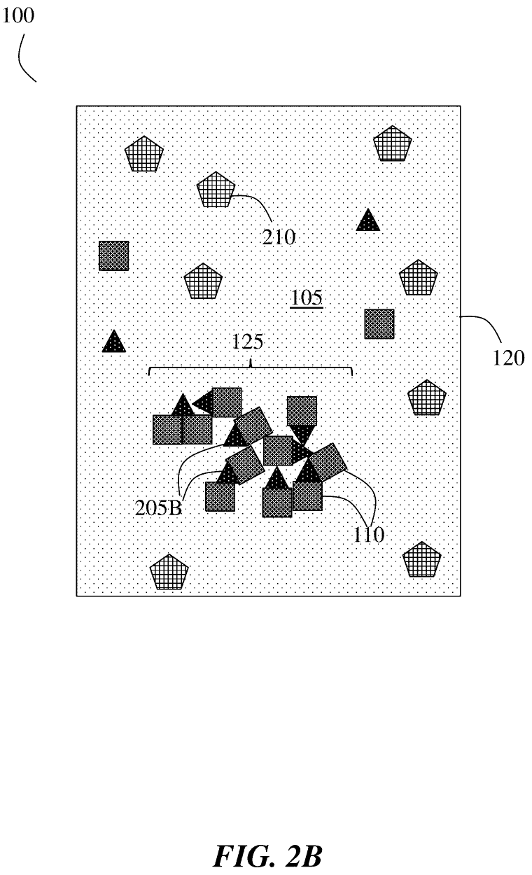

In some embodiments, the fluid comprises a polypeptide. For example, in FIG. 1A, system 100 comprises fluid 105 comprising polypeptide 115A. In some embodiments, the fluid comprises one or more polypeptides. For example, in FIG. 2A, fluid 105 comprises first polypeptide 205A and second polypeptide 210A. As used herein, the term “polypeptide” refers to any polymeric compound comprising two or more amino acid monomers in sequence, joined in chain via one or more peptide bonds. In some embodiments, a polypeptide comprises a relatively long chain of amino acids (e.g., 25 or more amino acids). The polypeptide can have any known structure such as a secondary, tertiary, and/or quaternary structure. In some embodiments, the secondary, tertiary, and/or quaternary structure may be altered after denaturation, in which case the denatured polypeptide or fragments thereof is still considered to be a “polypeptide” in this disclosure. In some embodiments, each of the one or more polypeptides comprises a distinct amino acid sequence. That is, the one or more polypeptides may comprise an enzyme, a protein, and/or another polypeptide each having a distinct sequence of amino acids.

In some embodiments, one or more polypeptides comprises a polymer-degrading enzyme or a fragment thereof. That is, one or more polypeptides in the fluid may be a polymer-degrading enzyme. In some embodiments, one or more polypeptides is derived from the polymer-degrading enzyme. That is, one or more polypeptides may be related to the polymer-degrading enzyme but was altered in some manner. For example, the polymer-degrading enzyme may be denatured, and accordingly, the resulting denatured enzyme is a polypeptide and is derived from the polymer-degrading enzyme. In some embodiments, the polypeptide comprises a fragment of the polymer-degrading enzyme.

As used herein, the term “denature” refers to the act and/or the process of at least partially altering the three-dimensional configuration of a polypeptide. In some embodiments, denaturation of a polypeptide may result in it having a quaternary structure, tertiary structure, and/or secondary structure relative to its pre-denatured state. Any process and/or chemistry/biochemistry available to those of ordinary skill in the art can be used to denature a polypeptide in accordance with this disclosure, and those of ordinary skill in the art are aware of a wide variety of processes and chemistries/biochemistries and can determine appropriate conditions and/or processes that might be used. For example, a polypeptide maybe denatured upon exposure to various temperatures, pressures, stresses, radiation, pH, and/or other agents, such as detergents, that may at least in-part induce the denaturing of a polypeptide. As one example, a polypeptide can denature when the temperature of the polypeptide is at or exceeds the melting temperature of the polypeptide. Those of ordinary skill in the art can readily determine the melting temperature of a polypeptide using any of a variety of method available in the art. In some cases, the solubility of a polypeptide may be affected upon denaturation. Denaturation of the polypeptide can in some cases result in partial or even full loss of function (e.g., enzymatic activity) of the polypeptide, but this is not a necessary outcome in all cases.

In some embodiments, at least one of the polypeptides, prior to denaturation, is a polymer-degrading enzyme. Without wishing to be bound by any particular theory, the polymer-degrading enzyme may facilitate the degradation of the plurality of particles (e.g., plastic particles) into one or more degradation products that may have commercial value. Accordingly, it may be necessary to remove the polymer-degrading enzyme and/or other polypeptides in the fluid to isolate the degradation products. In some embodiments, the polymer-degrading enzymes can be denatured to form a denatured polypeptide such that a mixture comprising the denatured polypeptide and at least some of the plurality of particles are separated from the fluid. For example, in FIG. 1A, fluid 105 comprises polypeptide 115A and plurality of particles 110 suspended in fluid 105, but once polypeptide 115A is denatured to form denatured polypeptide 115B, as shown in FIG. 1B, plurality of particles 110 associate with denatured polypeptide 115B to form agglomerate 125 such that agglomerate 125 settles in the fluid.

In some embodiments, the one or more polypeptides comprise a sacrificial polypeptide. In some embodiments, the sacrificial polypeptide has a lower melting temperature than the melting temperature of the polymer-degrading enzyme. Without wishing to be bound by any particular theory, the sacrificial polypeptide may allow for the separation of the plurality of particles from the fluid without altering and/or imparting damage onto the polymer-degrading enzyme and/or another polypeptide in the fluid. Accordingly, the polymer-degrading enzyme and/or other polypeptides may advantageously be reused and/or repurposed. By having a melting temperature lower than the melting temperature of the polymer-degrading enzyme, the sacrificial polypeptide may be denatured without altering the polymer-degrading enzyme and/or another polypeptide in the fluid as the sacrificial polypeptide may be denatured at a temperature lower than the melting temperature of the polymer-degrading enzyme. For example, in FIG. 2A, first polypeptide 205A, acting as the sacrificial polypeptide in this example, has a lower melting temperature than second polypeptide 210. Accordingly, when fluid 105 is heated to a temperature greater than the melting temperature of first polypeptide 205A but lower than that of second polypeptide 210, as shown in FIG. 2B, first polypeptide 205A denatures into denatured polypeptide 205B and agglomerates with plurality of particles 110 to form agglomerate 125. However, second polypeptide 210 is not altered nor denatured and remains in fluid 105. In some embodiments, the one or more polypeptides comprise the sacrificial polypeptide and the polymer-degrading enzyme. In some embodiments, the sacrificial polypeptide comprises bovine serum albumin and/or ovalbumin. In some embodiments, the one or more polypeptides comprise amphipathic helixes, heat shock proteins, chaperones, ribosomal proteins, albumin, collagen, fibronectin, hydrophobins, carbohydrate binding proteins, and/or lipases.

The sacrificial polypeptide described herein may have any of a variety of suitable properties. In some embodiments, the sacrificial polypeptide has a melting temperature. In some embodiments, the sacrificial polypeptide has a melting temperature greater than or equal to 50 degrees Celsius, greater than or equal to 55 degrees Celsius, greater than or equal to 60 degrees Celsius, greater than or equal to 65 degrees Celsius, greater than or equal to 70 degrees Celsius, greater than or equal to 75 degrees Celsius, greater than or equal to 80 degrees Celsius, greater than or equal to 85 degrees Celsius, greater than or equal to 90 degrees Celsius, or greater than or equal to 95 degrees Celsius. In some embodiments, the sacrificial polypeptide has a melting temperature less than or equal to 95 degrees Celsius, less than or equal to 90 degrees Celsius, less than or equal to 85 degrees Celsius, less than or equal to 80 degrees Celsius, less than or equal to 75 degrees Celsius, less than or equal to 70 degrees Celsius, less than or equal to 65 degrees Celsius, less than or equal to 60 degrees Celsius, less than or equal to 55 degrees Celsius, or less than or equal to 50 degrees Celsius. Combinations of these ranges are also possible (e.g., greater than or equal to 50 degrees Celsius and less than or equal to 95 degrees Celsius). Other ranges are also possible.

In some embodiments, the melting temperature of sacrificial polypeptide is relatively lower than the melting temperature of the polymer-degrading enzyme. In some embodiments, the melting temperature of the sacrificial polypeptide is at least 1 degree Celsius, at least 2 degrees Celsius, at least 3 degrees Celsius, at least 4 degrees Celsius, at least 5 degrees Celsius, at least 10 degrees Celsius, at least 15 degrees Celsius, at least 20 degrees Celsius, at least 30 degrees Celsius, at least 40 degrees Celsius, or at least 50 degrees Celsius lower than the melting temperature of the polymer-degrading enzyme.

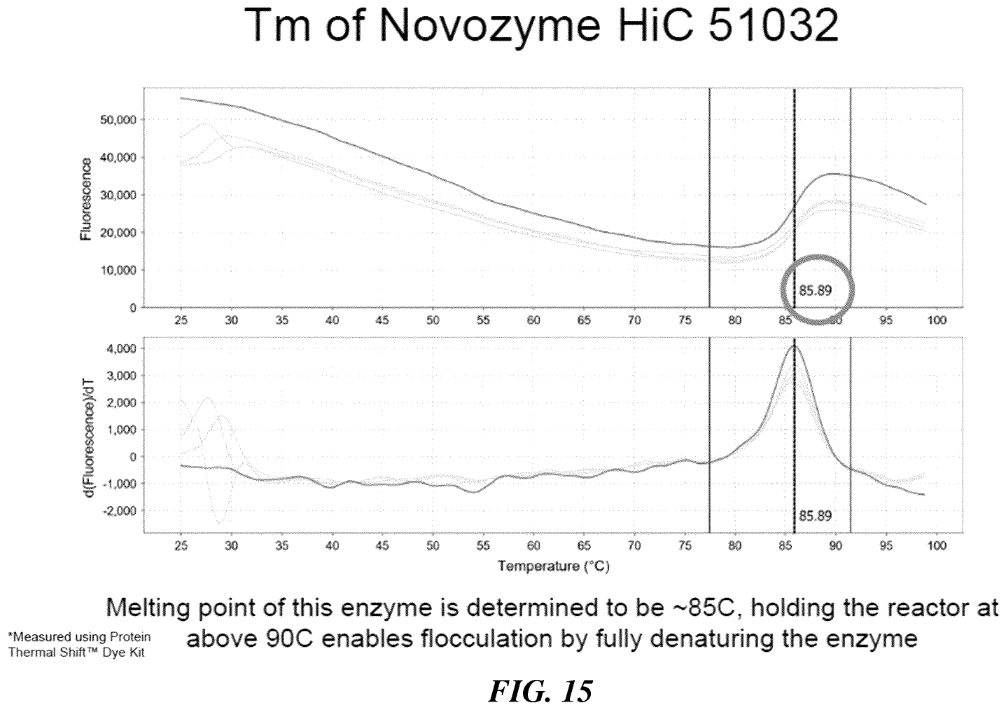

The melting temperature of polypeptides disclosed herein may be determined by any of a variety of methods known to those of ordinary skill in the art. For example, the melting temperature of the one or more polypeptides may be determined using a ThermoFisher Protein Thermal Shift Starter Kit. Accordingly, by determining the melting temperature of sacrificial polypeptide candidates and the polymer-degrading enzyme, one would be able to determine whether any of the sacrificial polypeptide candidates can successfully denature upon exposure to temperatures exceeding its melting temperature without affecting the polymer-degrading enzyme. An example of the melting temperature determination of an enzyme is shown in FIG. 15.

In some embodiments, at least some of the polypeptides comprise a hydrophilic portion. In some embodiments, at least some of the polypeptides comprise a hydrophobic portion. Hydrophobic and hydrophilic are defined herein in relation to the relative hydrophilic portions of a polypeptide compared with the polypeptide's hydrophobic portions, and how those portions interact with the aqueous fluid and/or a plastic particle. Polypeptides can self-assemble or fold, in their native state, into an arrangement in which the hydrophobic portions are largely internal when in an aqueous solution and the hydrophilic portions are largely exposed, rendering them soluble in an aqueous fluid. When denatured, more hydrophobic portions are exposed and can associate with a relatively hydrophobic plastic particle due to their likeness to it. These relationships are clearly understood and can easily be observed by those of ordinary skill in the art.

In some embodiments, the denatured polypeptide comprises an exposed hydrophobic portion. Without wishing to be bound by any particular theory, upon denaturation, the denatured polypeptide has an exposed hydrophobic portion that is associated with some of the plurality of particles. As described elsewhere in this disclosure, the plurality of particles may comprise one or more hydrophobic portions, and accordingly, such portions may have an affinity to the exposed hydrophobic portions of the denatured polypeptide. For example, in FIG. 1B, agglomerate 125 comprises denatured polypeptide 115B associated with plurality of particles 110 via affinity between a hydrophobic portion of denature polypeptide 115B and plurality of particles 110. However, prior to denaturation, in some embodiments, the hydrophilic portion of the one or more polypeptides may allow for them to disperse in solution and/or be suspended in the fluid without settling (e.g., precipitating) in the fluid.

In this disclosure, a polypeptide is associated with a particle when the two are attracted to each other to the extent that some of them, or a majority or even essentially all of them will be readily separated from a fluid or liquid in which the particles had previously resided (e.g., had been in suspension), as described elsewhere herein. The polypeptide and the particle or particles may be attracted to each other via any of a variety of interaction mechanisms including but not limited to chemical interactions (e.g., covalent interactions) and/or physical interactions (e.g., adsorption). In one set of embodiments, denatured polypeptides may associate with particles and facilitate their removal from a fluid via non-covalent interaction of the relatively hydrophobic portion of the polypeptide, exposed in its denatured form, with the particles.

In some embodiments, at least some of the plurality of particles are coupled to the hydrophobic portion of at least some of the polypeptides. In some embodiments, at least some of the plurality of particles are coupled to the hydrophobic portion of the denatured polypeptide. As used herein, “coupled to” may refer to covalent interactions, non-covalent interactions (e.g., van der waals interactions, electrostatic interactions), and/or adsorption. Without wishing to be bound by any particular theory, upon denaturation, the hydrophobic portion of the denatured polypeptide may have an affinity to some of the plurality of particles, and accordingly, form an agglomerate that may settle (e.g., precipitate) in the fluid. The agglomerate, in some embodiments, comprises one or more denatured polypeptides coupled to some of the plurality of particles.

In some embodiments, one or more polypeptides in the fluid may undergo at least partial denaturation. In some embodiments, when one or more of the polypeptides undergo at least partial denaturation, separation of the mixture comprising the plurality of particles and the denatured polypeptide from the fluid may commence. That is, at least some of the plurality of particles may associate with the denatured polypeptide upon its denaturation and settle (e.g., precipitate) in the fluid. Accordingly, the onset of separation may be related to the denaturation of at least one of the one or more polypeptides in the fluid. In some embodiments, a portion of one or more polypeptides in the fluid is denatured. In other words, when one or more polypeptides are exposed to conditions that may induce denaturation, some, and in some cases all, of the secondary, tertiary, and/or quaternary structure may be altered.

In some embodiments, the onset of separation is associated with exposing the fluid and/or the one or more polypeptides to a temperature greater than or equal to the melting temperature of at least one of the one or more polypeptides. As discussed elsewhere in the present disclosure, exposing the polypeptide to a temperature greater than or equal to its melting temperature may result in the quaternary, tertiary, or secondary structure of the polypeptide to be altered and in some cases, change any of a myriad of properties of the polypeptide including, but not limited to, its solubility in a fluid. Accordingly, the denatured polypeptide may settle (e.g., precipitate) out of the solution along with some of the plurality of particles associated with the denatured polypeptide. In some embodiments, denaturing one or more polypeptides may involve heating (e.g., to a temperature greater than the melting temperature of at least one of the polypeptides) and/or agitating the fluid comprising the one more polypeptides such that at least some of the polypeptides undergo denaturation. In some embodiments, denaturing the polypeptide involves heating the fluid comprising the one or more polypeptides to a relatively high temperature (e.g., to a temperature greater than the melting temperature of at least one of the polypeptides) and maintaining the temperature for a duration of time.

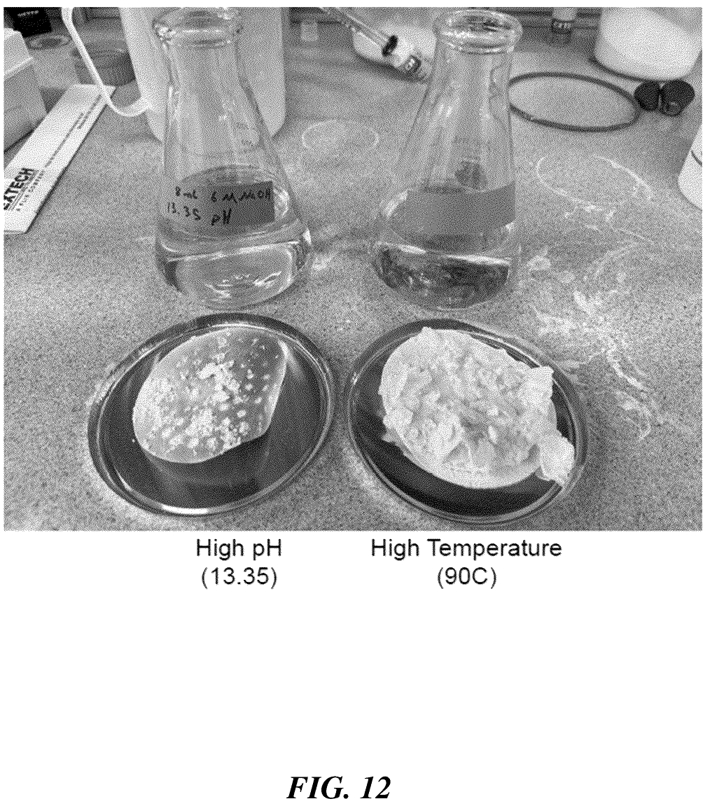

In some embodiments, at least partially denaturing the polypeptide comprises heating the fluid comprising the one or more polypeptides to a relatively high temperature (see FIG. 12). In some embodiments, the temperature of the fluid comprising the one or more polypeptides may be greater than or equal to the melting temperature of the one or more polypeptides. In some embodiments, the temperature of the fluid is greater than or equal to 50 degrees Celsius, greater than or equal to 55 degrees Celsius, greater than or equal to 60 degrees Celsius, greater than or equal to 65 degrees Celsius, greater than or equal to 70 degrees Celsius, greater than or equal to 75 degrees Celsius, greater than or equal to 80 degrees Celsius, greater than or equal to 85 degrees Celsius, greater than or equal to 90 degrees Celsius, or greater than or equal to 95 degrees Celsius. In some embodiments, the temperature of the fluid is less than or equal to 95 degrees Celsius, less than or equal to 90 degrees Celsius, less than or equal to 85 degrees Celsius, less than or equal to 80 degrees Celsius, less than or equal to 75 degrees Celsius, less than or equal to 70 degrees Celsius, less than or equal to 65 degrees Celsius, less than or equal to 60 degrees Celsius, less than or equal to 55 degrees Celsius, or less than or equal to 50 degrees Celsius. Combinations of these ranges are also possible (e.g., greater than or equal to 50 degrees Celsius and less than or equal to 95 degrees Celsius). Other ranges are also possible.

In some embodiments, the relatively high temperature may be maintained for any of a variety of durations. In some embodiments, the temperature of the fluid is maintained for a duration greater than or equal to 1 minute, greater than or equal to 5 minutes, greater than or equal to 10 minutes, greater than or equal to 15 minutes, greater than or equal to 20 minutes, greater than or equal to 25 minutes, greater than or equal to 30 minutes, greater than or equal to 45 minutes, greater than or equal to 60 minutes, or greater than or equal to 2 hours. In some embodiments, the temperature of the fluid is maintained for a duration less than or equal to 2 hours, less than or equal to 60 minutes, less than or equal to 45 minutes, less than or equal to 30 minutes, less than or equal to 25 minutes, less than or equal to 20 minutes, less than or equal to 15 minutes, less than or equal to 10 minutes, less than or equal to 5 minutes, or less than or equal to 1 minute. Combinations of these ranges are also possible (e.g., greater than or equal to 1 minute and less than or equal to 2 hours). Other ranges are also possible.

In some embodiments, the onset of separation is associated with exposing one or more of the polypeptides to radiation. That is, one or more polypeptides in the fluid may denature upon exposure to radiation, including but not limited to electromagnetic radiation (e.g., infrared radiation and/or ultraviolet radiation). In some embodiments, at least partially denaturing the polypeptide in the fluid comprises exposing the polypeptide to electromagnetic radiation. In some embodiments, the electromagnetic radiation comprises infrared radiation and/or ultraviolet radiation. In some embodiments, the one or more polypeptides in the fluid can be exposed to the radiation for at least 30 seconds, at least 1 minutes, at least 5 minutes, at least 10 minutes, at least 20 minutes, at least 30 minutes, at least 1 hours, or at least 2 hours. In some embodiments, the electromagnetic radiation has a wavelength of greater than or equal to 100 nm and less than or equal to 400 nm. In some embodiments, the electromagnetic radiation has a wavelength of greater than or equal to 780 nm and less than or equal to 1 mm.

In some embodiments, the onset of separation is associated with applying a relatively high pressure to the polypeptide. In some embodiments, the onset of separation is associated with applying a relatively high pressure to a fluid, some of which may be transferred to the polypeptide. In some embodiments, at least partially denaturing the polypeptide in the fluid comprises applying a relatively high pressure to the polypeptide and/or fluid. That is, the polypeptide may denature upon the application of sufficient pressure to alter the quaternary, tertiary, and/or secondary structure of the polypeptide.

Any of a variety of suitable pressures may be applied to the polypeptide such that it undergoes denaturation. In some embodiments, denaturing the polypeptide comprises applying a pressure of greater than or equal to 1 kbar, greater than or equal to 1.5 kbar, greater than or equal to 2 kbar, greater than or equal to 2.5 kbar, greater than or equal to 3 kbar, greater than or equal to 3.5 kbar, greater than or equal to 4 kbar, greater than or equal to 4.5 kbar, or greater than or equal to 5 kbar to the polypeptide and/or the fluid. In some embodiments, denaturing the polypeptide comprises applying a pressure of less than or equal to 5 kbar, less than or equal to 4.5 kbar, less than or equal to 4 kbar, less than or equal to 3.5 kbar, less than or equal to 3 kbar, less than or equal to 2.5 kbar, less than or equal to 2 kbar, less than or equal to 1.5 kbar, or less than or equal to 1 kbar, to the polypeptide and/or the fluid. Combinations of these ranges are also possible (e.g., greater than or equal to 1 kbar and less than or equal to 5 kbar). Other ranges are possible.

In some embodiments, onset of separation is associated with exposing the polypeptide in the fluid to a relatively high and/or a relatively low pH. In some embodiments, at least partially denaturing the polypeptide in the fluid comprises exposing the polypeptide in the fluid to a relatively high and/or a relatively low pH. That is, the polypeptide, when exposed to a relatively high and/or relatively low pH, may undergo denaturation.

In some embodiments, denaturing the polypeptide comprises exposing the polypeptide in the fluid to a relatively high pH (see FIG. 12). In some embodiments, one or more of the polypeptides in the fluid are exposed to a pH greater than or equal to 12, greater than or equal to 12.5, greater than or equal to 13, greater than or equal to 13.5, greater than or equal to 14. In some embodiments, one or more of the polypeptides in the fluid are exposed to a pH less than or equal to 14, less than or equal to 13.5, less than or equal to 13, less than or equal to 12.5, or less than or equal to 12. Combinations of these ranges are possible (e.g., greater than or equal to 12 and less than or equal to less than or equal to 14). Other ranges are also possible.

In some embodiments, denaturing the polypeptide comprises exposing the polypeptide in the fluid to a relatively low pH. In some embodiments, one or more of the polypeptides in the fluid are exposed to a pH less than or equal to 2, less than or equal to 1.5, less than or equal to 1, or less than or equal to 0.5. In some embodiments, one or more of the polypeptides in the fluid are exposed to a pH greater than or equal to 0.5, greater than or equal to 1, greater than or equal to 1.5, or greater than or equal to 2. Combinations of these ranges are possible (e.g., less than or equal to 2 and greater than or equal to 0.5). Other ranges are also possible.

In some embodiments, the onset of separation is associated with exposing the polypeptide in the fluid to a shear strain at a shear rate. In some embodiments, at least partially denaturing the polypeptide in the fluid comprises exposing the polypeptide in the fluid to a shear strain at a shear rate. Without wishing to be bound by any particular theory, the quaternary, tertiary, and/or secondary structure of the polypeptide may be altered upon exposure to relatively high shear rates, and accordingly, the polypeptide may undergo denaturation. In some embodiments, the shear rate may be imparted by a high shear mixer such as an inline high-shear mixer and/or an ultra-high-shear inline mixer. Examples of such mixers include but are not limited to a IKA DISPAX-REACTOR® DRS and/or a ROSS 700 series.

Any of a variety of shear rates may be applied to the polypeptide. In some embodiments, the shear rate applied to the polypeptide is greater than or equal to 1000 s⁻¹, greater than or equal to 5000 s⁻¹, greater than or equal to 10000 s⁻¹, greater than or equal to 50000 s⁻¹, greater than or equal to 100000 s⁻¹, greater than or equal to 500000 s⁻¹. In some embodiments, the shear rate applied to the polypeptide is less than or equal to 500000 s⁻¹, less than or equal to 100000 s⁻¹, less than or equal to 50000 s⁻¹, less than or equal to 10000 s⁻¹, less than or equal to 5000 s⁻¹, less than or equal to 1000 s⁻¹. Combinations of these ranges are also possible (e.g., greater than or equal to 1000 s⁻¹ and less than or equal to 500000 s⁻¹). Other ranges are also possible.

In some embodiments, the onset of separation is associated with exposing the polypeptide to the fluid having a relatively high salt concentration. In some embodiments, at least partially denaturing the polypeptide comprises exposing the polypeptide to the fluid having a relatively high salt concentration. That is, the polypeptide in the fluid may, in the presence of a fluid having a relatively high salt concentration, undergo denaturation. In some embodiments, the fluid has a salt concentration greater than or equal to 1 M, greater than or equal to 1.5 M, greater than or equal to 2 M, greater than or equal to 2.5 M, greater than or equal to 5 M, greater than or equal to 10 M. In some embodiments, the fluid has a salt concentration less than or equal to 10 M, less than or equal to 5 M, less than or equal to 2.5 M, less than or equal to 2 M, less than or equal to 1.5 M, or less than or equal to 1 M. Combinations of these ranges are possible (e.g., greater than or equal to 1 M and less than or equal to 10 M). Other ranges are possible.

In some embodiments, the onset of separation is associated with the fluid coming to rest after exposure of some of the polypeptide to any of the variety of conditions described throughout this disclosure. Without wishing to be bound by any particular theory, after exposing some of the polypeptides to conditions that induce denaturation (e.g., temperatures greater than or equal to the melting temperature of the polypeptides) it may be advantageous to allow the fluid to rest (e.g., without imparting any disturbances such as agitation) so that some of the polypeptides may associate with some of the plurality of particles. As described elsewhere in this disclosure, the fluid may be agitated during denaturation of some of the polypeptides which may inhibit the association between the denatured polypeptides and some of the plurality of the particles. In some embodiments, the onset of separation is associated with the fluid resting for greater than or equal to 30 seconds, greater than or equal to 1 minutes, greater than or equal to 5 minutes, greater than or equal to 10 minutes, greater than or equal to 20 minutes, greater than or equal to 30 minutes, greater than or equal to 40 minutes, greater than or equal to 50 minutes, greater than or equal to 60 minutes, greater than or equal to 90 minutes, or greater than or equal to 120 minutes. In some embodiments, the onset of separation is associated with the fluid resting for less than or equal to 120 minutes, less than or equal to 90 minutes, less than or equal to 60 minutes, less than or equal to 50 minutes, less than or equal to 40 minutes, less than or equal to 30 minutes, less than or equal to 20 minutes, less than or equal to 10 minutes, less than or equal to 5 minutes, less than or equal to 1 minute, or less than or equal to 30 seconds. Combinations of these ranges are possible (e.g., greater than or equal to 30 seconds and less than or equal to 120 minutes). Other ranges are also possible.

In some embodiments, the fluid comprises the plurality of particles. The plurality of particles may have any of a myriad of shapes including but not limited spheres, flakes, and/or platelets. In some embodiments, the plurality of particles may undergo a milling process prior to their introduction into the fluid. In some embodiments, the plurality of particles can be non-homogenous in composition, size, and/or shape. In some embodiments, the plurality of particles can be relatively homogeneous in composition, size, and/or shape. Without wishing to be bound by any particular theory, by using a plurality of particles as a feedstock for enzymatic degradation applications, the rate of enzymatic degradation may be relatively high. However, the plurality of particles may not be completely degraded after the enzymatic degradation process. The average maximum dimension of the partially degraded particles may be relatively small and may be suspended in the fluid. Accordingly, their removal of these suspended particles from the fluid is important to be able to isolate the degradation products that are in the fluid.

In some embodiments, the plurality of particles comprises a plurality of plastic particles. As used herein, the term “plastic” is given its ordinary meaning in the art. A “plastic” is a material or materials the makeup of which will be clearly understood by those of ordinary skill in the art. In typical embodiments, plastics comprise a polymeric material. In some embodiments, plastic comprise additives which include but are not limited to fillers, pigments, and/or antioxidants. In some embodiments, plastics comprise one or more crystalline domains. In some embodiments, plastics comprise one or more amorphous domains. In some embodiments, plastics are crystalline. In some embodiments, plastics are amorphous. In some embodiments, plastics are a synthetic material. In some embodiments, plastics are a synthetic material comprising one or more organic polymers. In some embodiments, the plurality of particles comprises a plurality of polymeric particles. In some embodiments, plastics are semi-crystalline.

The plurality of plastic particles may comprise any of a variety of plastics. In some embodiments, the plurality of plastic particles comprises a polyester, polyamide, polyolefin, polystyrene (e.g., syndiotactic polystyrene), fluoropolymer, polyurethane, polyether ether ketone, semi-crystalline thermoplastic polyurethane, substituted forms of the foregoing, and/or combinations thereof. In some embodiments, the plurality of plastic particles comprise polyethylene terephthalate. In some embodiments, the plurality of plastic particles comprise polyethylene terephthalate (PET), poly (lactic acid) (PLA), poly (L-lactic acid) (PLLA), poly (D-lactic acid) (PDLA), polybutylenesuccinate (PBS), polycaprolactone (PCL), poly (ethylenc adipate), polybutylene terephthalate (PBT), and/or combinations thereof. Examples of polyamides include, but are not limited to, polyamide 6, poly (beta-caprolactam), polycaproamide, polyamide-6,6, poly (hexamethylene adipamide) (PA6,6), poly (11-aminoundecanoamide) (PA11), polydodecanolactam (PA12), poly (tetramethylene adipamide) (PA4,6), poly (pentamethylene sebacamide) (PA6,10), poly (hexamethylene dodecanoamide) (PA6,12), poly (m-xylylencadipamide) (PAMXD6), polyhexamethylene adipamide/polyhexamethylene terephthalamide copolymer (PA66/6T), polyhexamethylene adipamide/polyhexamethylene isophthalamide copolymer (PA66/61), and/or combinations thereof. In some embodiments, the plurality of plastic particles comprise polyethylene (e.g., high-density polyethylene, medium-density polyethylene, linear low-density polyethylene, very-low-density polyethylene, etc.), polypropylene, isotactic polypropylene, syndiotactic polypropylene, and/or combinations thereof. In some embodiments, the plurality of plastic particles comprises polyurethanes including but not limited to elastane (e.g., polyether and polyurea copolymers, Spandex, Lycra).

In some embodiments, the plurality of particles comprises a post-consumer/industrial polymeric material (PC/IPM). “PC/IPM” is a material or materials the makeup of which will be clearly understood by those of ordinary skill in the art. In typical embodiments, such material or materials are polymers that have been formed for a particular use, such as consumer and/or industrial products or processes, then identified for a subsequent transformation, process, reaction, or interaction, such as recycling. A post-consumer and/or post-industrial polymeric material (PC/IPM) may be or may include a manufacturing or compounding scrap or manufactured objects that were never sold to and/or never used by consumers. Post-consumer and/or post-industrial polymeric materials (post-consumer/industrial polymeric materials; PC/IPMs) have generally been a challenging class of materials to recycle. Typically, PC/IPMs include a myriad of polymeric materials (e.g. polymers and/or polymer-based composites, etc). In one set of embodiments, PC/IPMs are polymeric materials generated by households, and/or by commercial, institutional, and/or industrial entities in their role as end or intermediate users of products which can no longer be used or are undesirable for its intended purpose. A PC/IPM can be a polymer material diverted during the manufacturing or commercial process. For example, such materials can be polymers and/or copolymers that have been formed for a particular use, then identified for a subsequent transformation, process, reaction, or interaction, such as recycling.

In some embodiments, the plurality of particles comprise a virgin polymeric material. “Virgin polymeric material” is a polymeric material that has been produced from petrochemical feedstock (e.g., crude oil, natural gas) and has not been further processed or used to form a consumer or industrial object or product (e.g., a PC/IPM). Those of ordinary skill in the art will understand that virgin polymeric material may comprise one or more additives (e.g., catalysts). A virgin plastic and/or a virgin polymeric material generally refers to a polymeric material that has been produced directly from petrochemical feedstock (e.g., crude oil, natural gas) and has not been previously used or processed (e.g., processed into a consumer or industrial product, used in an industrial process). In some embodiments, a virgin plastic and/or polymeric material can be produced from at least a portion of biomass feedstock. In some embodiments, virgin polymeric materials comprises crystallizable polymers or copolymers in virgin form. A virgin plastic and/or a virgin polymeric material is a material the makeup of which is well understood by those of ordinary skill in the art. A virgin plastic, in certain cases, may comprise some amount (if any) of additives (e.g., catalysts, antioxidants, unreacted monomers, plasticizers, etc.) and comprise crystallizable polymers or copolymers containing some comonomers. The post-consumer and/or post-industrial polymeric material, in certain cases, may comprise some amount of additives (e.g., polymers, small molecules such as but not limited to processing aids, dyes, antioxidants, pigments, fillers, etc.) incorporated into the virgin plastic. In some cases, the virgin polymeric material comprises one or more additives (e.g., catalysts, dyes, contaminants, lubricants, etc).

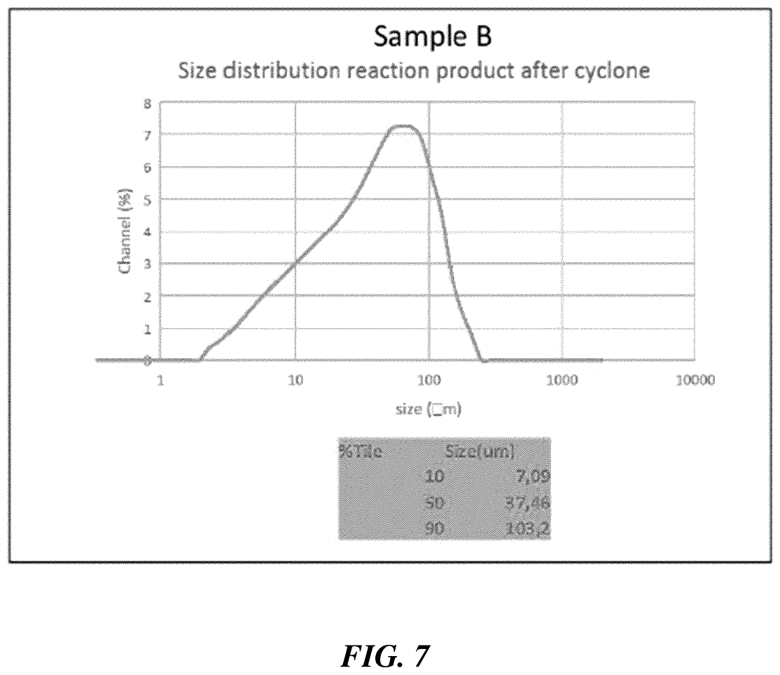

The plurality of particles in the fluid may have any of a variety of suitable sizes. Without wishing to be bound by any particular theory, after the plurality of particles are exposed to the polymer-degrading enzyme, the average maximum dimension of the plurality of particles may decrease as they are degraded. Accordingly, the plurality of particles in the fluid may be relatively small (FIG. 7). In some embodiments, the plurality of particles in the fluid have an average maximum dimension greater than or equal to 50 micrometers, greater than or equal to 100 micrometers, greater than or equal to 150 micrometers, greater than or equal to 200 micrometers, greater than or equal to 300 micrometers, greater than or equal to 400 micrometers, greater than or equal to 500 micrometers, greater than or equal to 1 mm, and greater than or equal to 2 mm. In some embodiments, the plurality of particles in the fluid have an average maximum dimension less than or equal to 2 mm, less than or equal to 1 mm, less than or equal to 500 micrometers, less than or equal to 400 micrometers, less than or equal to 300 micrometers, less than or equal to 200 micrometers, less than or equal to 150 micrometers, less than or equal to 100 micrometers, and less than or equal to 50 micrometer. Combinations of these ranges (e.g., greater than or equal to 50 micrometers and less than or equal to 2 mm). Other ranges are also possible.

In some embodiments, the plurality of plastic particles comprise microplastics. In some embodiments, the microplastics comprise plastic particles having an average maximum dimension greater than or equal to 1 nm and less than or equal to 5 mm. according to the United States Environmental Protection Agency (https://www.epa.gov/water-research/microplastics-research). Accordingly, it is important to note that while the present disclosure discusses the separation of a plurality of particles from a fluid for applications related to enzymatic degradation, it is not intended to be limiting in this regard. The methods and compositions described in the totality of this disclosure can, in some embodiments, may be related to the other applications such as the separation of microplastics and/or other contaminants from fluids (e.g., potable and/or non-potable water sources).

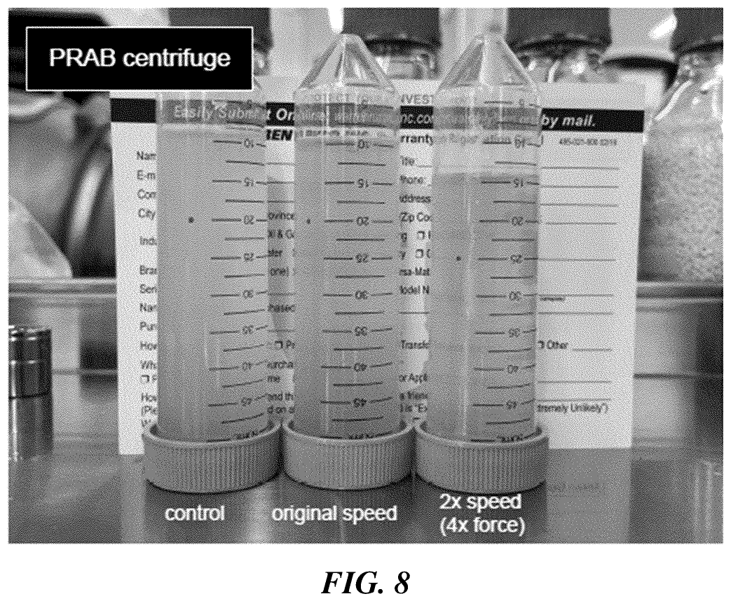

In some embodiments, the plurality of particles are suspended in the fluid. For example, in FIG. 1A and FIG. 2A, plurality of particles 110 are suspended in fluid 105. Without wishing to be bound by any particular theory, after enzymatic degradation, the suspended plurality of particles have a relatively small size and are difficult to separate from the fluid with filtration and centrifugation techniques (see FIG. 8). Accordingly, by denaturing the polypeptide in the fluid, the plurality of particles and the fluid are separated as the plurality of particles associate with the denatured polypeptide.

In some embodiments, a portion of some of the plurality of particles are hydrophobic. Accordingly, the hydrophobic portions of some of the plurality of particles may have an affinity for the hydrophobic portions of the denatured polypeptide and thereby associate with the denatured polypeptide to form one or more agglomerates. The fluid and the agglomerates may then be separated from each other through a filtration process as the plurality of particles, being associated with the denatured polypeptide, may not clog the filter nor inhibit filtration (see FIG. 11). Therefore, the filtration process may proceed at advantageous rates and/or with advantageous throughputs relevant for enzymatic degradation processes at commercial scales.

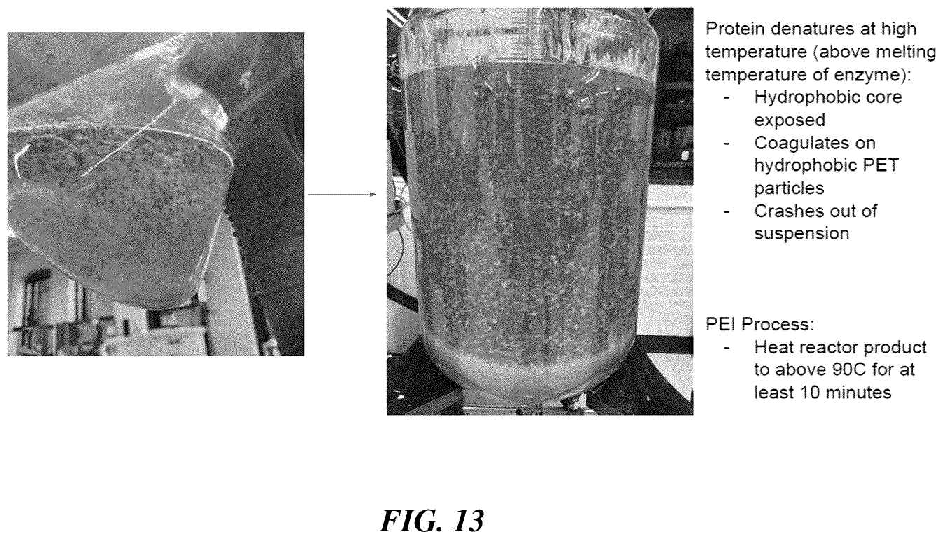

In some embodiments, the plurality of particles, in the absence of the denatured polypeptide, are suspended in the fluid. For example, in FIG. 1A, plurality of particles 110 are suspended in fluid 105 as plurality of particles 110 are not in the presence of a denatured polypeptide. In some embodiments, the plurality of particles agglomerate and settle in the fluid when in the presence of the denatured polypeptide. As an example, in FIG. 1B, plurality of particles 110 are in the presence of and are associated with denatured polypeptide 115B, and accordingly, plurality of particles 110 agglomerate to form agglomerate 125 that settles (e.g., precipitates) in fluid 105. However, it is important to note that the agglomerate comprising some of the plurality of particles and the denatured polypeptide may be suspended (e.g., float) in the fluid and/or settle (e.g., precipitate) in the fluid but may still be regarded as separate from the fluid, and the figures described herein are not intended to be limiting in such a manner. As an example, in FIG. 13, while some agglomerates, comprising the denatured polypeptide and some of the plurality of particles, are settled (e.g., precipitated) in the fluid and some agglomerates are suspended (e.g., floating) in the fluid, the agglomerates described in this example may still be considered to be separate from the fluid.

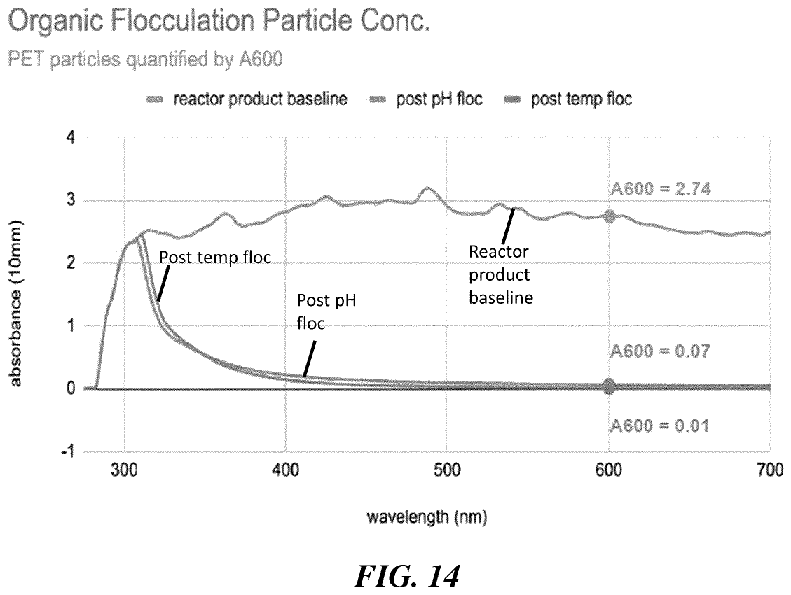

In some embodiments, the concentration of the plurality of particles in the fluid after separation is relatively low. As an example, in FIG. 14, UV-VIS spectroscopy was used to determine to determine the relative concentration the plurality of particles in the fluid and shows that the relative concentration of the plurality of particles in the fluid was less after separation (induced by both a relatively high temperature and a relatively high pH). In some embodiments, the plurality of particles is present in the fluid after separation in an amount less than or equal to 2 wt %, less than or equal to 1.8 wt %, less than or equal to 1.6 wt %, less than or equal to 1.4 wt %, less than or equal to 1.2 wt %, less than or equal to 1.0 wt %, less than or equal to 0.8 wt %, less than or equal to 0.6 wt %, less than or equal to 0.4 wt %, or less than or equal to 0.2 wt %. In some embodiments, the plurality of particles is present in the fluid after separation in an amount greater than or equal to 0 wt %, greater than or equal to 0.2 wt %, greater than or equal to 0.4 wt %, greater than or equal to 0.6 wt %, greater than or equal to 0.8 wt %, greater than or equal to 1.0 wt %, greater than or equal to 1.2 wt %, greater than or equal to 1.4 wt %, greater than or equal to 1.6 wt %, greater than or equal to 1.8 wt %, or greater than or equal to 2 wt %. Combinations of these ranges are possible (e.g., less than or equal to 2 wt % and greater than or equal to 0 wt %). Other ranges are also possible.

In some embodiments, the methods described herein are related to the separation of the plurality of particles from the fluid. In some embodiments, the fluid comprises an aqueous fluid (e.g., water). In some embodiments, the fluid comprises one or more polypeptides. In some embodiments, the fluid comprises the polymer-degrading enzyme. In some embodiments, the fluid comprises a plurality of particles.

Without wishing to be bound by any particular theory, the fluid may serve as a medium for enzymatic degradation of a substrate (e.g., the plurality of particles). As the polymer-degrading enzyme degrades (e.g., depolymerizes) the plurality of particles, monomeric and/or oligomeric molecules derived from the plurality of particles may be present in the fluid. As an example, when exposing PET to the polymer-degrading enzyme, degradation products such as terephthalic acid, ethylene glycol, bis (2-hydroxylethyl) terephthalate (BHET), and/or mono (2-hydroxyethyl) terephthalate may be present in the fluid. These degradation products may be used (e.g., as precursors) to produce materials and/or for other applications that have commercial value. Therefore, by separating the plurality of particles from the fluid, degradation products that are substantially-free from the plurality of particles, the denatured polypeptide, and/or the one or more polypeptides may be desirable. Degradation products that are contaminated with relatively large amounts of the polypeptides and/or the plurality of particles may be undesirable for many commercial applications. Accordingly, in some embodiments, the fluid comprises one or more degradation products.

In some embodiments, the fluid, after separation, comprises a relatively low concentration of at least one of the polypeptides. In some embodiments, the fluid, after separation, comprises at least one of the polypeptides in a concentration less than or equal to 1×10⁻³ M, less than or equal to 5×10⁻⁴ M, less than or equal to 1×10⁻⁴ M, less than or equal 5×10⁻⁵ M, less than or equal to 1×10⁻⁵ M, less than or equal to 5×10⁻⁶ M, less than or equal to 1×10^{-6 M, less than or equal to} 5×10⁻⁷ M, and less than or equal to 1×10⁻⁷ M. In some embodiments, the fluid, after separation, comprises at least one of the polypeptides in a concentration greater than or equal to 1×10⁻⁷ M, greater than or equal to 5×10⁻⁷ M, greater than or equal to 1×10⁻⁶ M, greater than or equal to 5×10⁻⁶ M, greater than or equal to 1×10⁻⁵ M, greater than or equal to 5×10⁻⁵ M, greater than or equal to 1×10⁻⁴ M, greater than or equal to 5×10⁻⁴ M, greater than or equal to 1×10⁻³ M. Combinations of these ranges are also possible (e.g., less than or equal to 1×10⁻³ M and greater than or equal to 1×10⁻⁷ M). Other ranges are also possible.

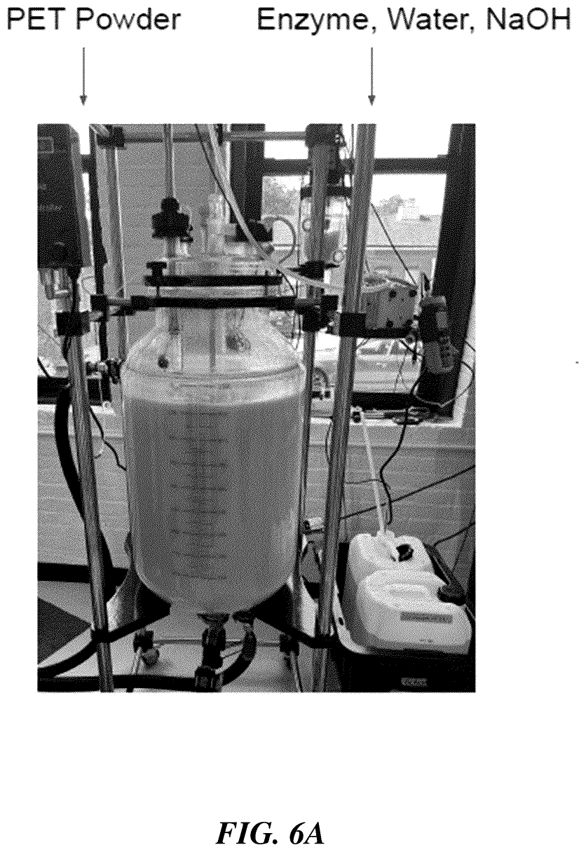

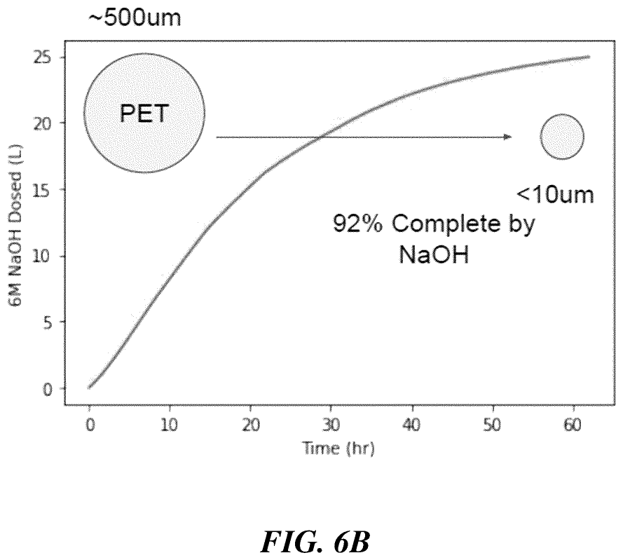

In some embodiments, the fluid may have any of a variety of properties that may facilitate the enzymatic degradation of the plurality of plastic particles. In some embodiments, the fluid comprises coenzymes (e.g., BHETase and/or MHETase), buffer solutions, salts, and/or other components that facilitate enzymatic degradation of the plurality of particles. For example, in FIG. 6A, PET particles are shown suspended in a fluid comprising enzymes, water, and sodium hydroxide. In some embodiments, sodium hydroxide or other dissociative basic compounds known in the art may be introduced into the fluid to maintain a pH level, and the amount of the compound dosed into the fluid may be indicative of reaction completion (see Example 1 and FIG. 6B).

In some embodiments, the fluid can be agitated (e.g., mixed) with a stirrer, an impeller, or the like. In some embodiments, the fluid may be agitated before and/or during the denaturation of the one or more polypeptides in the fluid. For example, as described elsewhere in this disclosure, some of the polypeptides in the fluid may be denatured upon exposure to a temperature greater than or equal to their melting temperature, and when increasing the temperature of the fluid to expose some of the polypeptides to a relatively high temperature, the fluid may be agitated. Such agitation can, in some embodiments, promote the uniform heating of the fluid and the polypeptides in the fluid. In some embodiments, the fluid may not be agitated during separation. In other words, after denaturation, the fluid may be allowed to rest (e.g., without exposing the fluid to any disturbances) such that some of the denatured polypeptides in the fluid can associate with some of the plurality of particles in the fluid. Without wishing to be bound by any particular theory, agitation of the fluid may inhibit and/or prevent some of the denatured polypeptide from associating with some of the plurality of particles and may further inhibit the separation of the plurality of particles from the fluid. Accordingly, in some embodiments, the fluid is not agitated during the separation of the mixture from the fluid.

In some embodiments, the fluid, after denaturation of some of the polypeptides in the fluid, may rest any of a variety of suitable durations. In some embodiments, the fluid, after denaturation of some of the polypeptides in the fluid, can rest for greater than or equal to 30 seconds, greater than or equal to 1 minute, greater than or equal to 5 minutes, greater than or equal to 10 minutes, greater than or equal to 20 minutes, greater than or equal to 30 minutes, greater than or equal to 40 minutes, greater than or equal to 50 minutes, greater than or equal to 60 minutes, greater than or equal to 90 minutes, or greater than or equal to 120 minutes. In some embodiments, the fluid, after denaturation of some of the polypeptides in the fluid, can rest for less than or equal to 120 minutes, less than or equal to 90 minutes, less than or equal to 60 minutes, less than or equal to 50 minutes, less than or equal to 40 minutes, less than or equal to 30 minutes, less than or equal to 20 minutes, less than or equal to 10 minutes, less than or equal to 5 minutes, less than or equal to 1 minute, less than or equal to 30 seconds. Combinations of these ranges are possible (e.g., greater than or equal to 10 minutes and less than or equal to 120 minutes). Other ranges are also possible.

The methods described herein generally relate to separating a mixture from the fluid. In some embodiments, the mixture comprises the plurality of particles and the denatured polypeptide. In some embodiments, the mixture comprises the plurality of particles and one or more denatured polypeptides. In some embodiments, the mixture comprises agglomerates comprising some of the plurality of particles and one or more denatured polypeptides. In some embodiments, the mixture is a product of the flocculation of one or more denatured polypeptides. That is, the denatured polypeptide may associate with some of the plurality of particles to form a mixture that coagulates and separates from the fluid.

In some embodiments, the mixture comprises some of the plurality of plastic particles in the fluid. In some embodiments, the mixture comprises greater than or equal to 10%, greater than or equal to 15%, greater than or equal to 20%, greater than or equal to 25%, greater than or equal to 30%, greater than or equal to 40%, greater than or equal to 50%, greater than or equal to 60%, or greater than or equal to 70% the total amount of plastic particles in the fluid. In some embodiments, the mixture comprises less than or equal to 70%, less than or equal to 60%, less than or equal to 50%, less than or equal to 40%, less than or equal to 30%, less than or equal to 25%, less than or equal to 20%, less than or equal to 15%, and less than or equal to 10% the total amount of plastic particles in the fluid. Combinations of these ranges are possible (e.g., greater than or equal to 10% and less than or equal to 70%). Other ranges are also possible.

In some embodiments, the mixture can be separated from a supernatant comprising the fluid. That is, the mixture comprising some of the plurality of particles and one or more denatured polypeptides may settle (e.g., precipitate) out of the fluid forming a supernatant. In some embodiments, the supernatant comprises the fluid. In some embodiments, the supernatant may include a portion of the plurality of particles.

In some embodiments, the supernatant comprises at least one of the polypeptides. In some embodiments, the concentration of at least one of the polypeptides in the supernatant is less than or equal to 1×10⁻³ M, less than or equal to 5×10⁻⁴ M, less than or equal to 1×10⁻⁴ M, less than or equal 5×10⁻⁵ M, less than or equal to 1×10⁻⁵ M, less than or equal to 5×10⁻⁶ M, less than or equal to 1×10⁻⁶ M, less than or equal to 5×10⁻⁷ M, and less than or equal to 1×10⁻⁷ M. In some embodiments, the concentration of at least one of the polypeptides in the supernatant is greater than or equal to 1×10⁻⁷ M, greater than or equal to 5×10⁻⁷ M, greater than or equal to 1×10⁻⁶ M, greater than or equal to 5×10⁻⁶ M, greater than or equal to 1×10⁻⁵ M, greater than or equal to 5×10⁻⁵ M, greater than or equal to 1×10⁻⁴ M, greater than or equal to 5×10⁻⁴ M, greater than or equal to 1×10⁻³ M. Combinations of these ranges are also possible (e.g., less than or equal to 1×10⁻³ M and greater than or equal to 1×10⁻⁷ M). Other ranges are also possible.

It is important to note that, throughout this disclosure, the separation of the mixture comprising some of the plurality of particles and the denatured polypeptide may not be related to isolation of the mixture. That is, for the mixture and the fluid to be separate, it is not necessary for them to be isolated from each other. In some embodiments, the mixture may be in contact with the fluid and separated from the fluid. In some embodiments, the mixture may be submerged in the fluid and separated from the fluid. In some embodiments, when the mixture and the fluid are separated, one or more domains may form wherein a first domain comprises the mixture and a second domain comprises the fluid. In some embodiments, the mixture is separated from the fluid without centrifugation and/or filtration. In some embodiments, separating the mixture from the fluid comprises filtration and/or centrifugation. Without wishing to be bound by any particular theory, filtration and/or centrifugation steps may be implemented with advantageously high throughputs after the denatured polypeptides associate with some of the plurality of particles and settle in the fluid.

After the mixture has been separated from the fluid, the mixture may be at least partially isolated from the fluid using any of a variety of methods to form a composition. That is, the composition, in some embodiments, is a product of at least partial isolation from a solution and/or a suspension comprising the plurality of particles (e.g., plastic particles) and one or more of the polypeptides. In some embodiments, the mixture may be isolated from the fluid via filtration. In some embodiments, the mixture may be isolated from the fluid via centrifugation. Those of ordinary skill in the art will clearly understand and know of suitable techniques that may be used to isolate the mixture from the fluid. In some embodiments, relatively small amounts of the fluid may be present within the composition. In some embodiments, the composition comprises a relatively small amount of the fluid in residual and/or trace amounts.

In some embodiments, the composition comprises one or more denatured polypeptides. As discussed elsewhere in this disclosure, the one or more denatured polypeptides may have undergone denaturation upon exposure to any of a variety of conditions (e.g., temperature, radiation, pressure, flocculation agents, etc.). In some embodiments, the composition comprises a denatured polypeptide comprising and/or derived from the polymer-degrading enzyme or a fragment thereof. In some embodiments, the presence of the denatured polypeptide comprising and/or derived from the polymer-degrading enzyme or a fragment thereof can indicate that the composition is a product from

In some embodiments, the composition comprises a relatively large amount of denatured polypeptides. In some embodiments, the one or polypeptides in the composition comprises greater than or equal to 50%, greater than or equal to 55%, greater than or equal to 60%, greater than or equal to 65%, greater than or equal to 70%, or greater than or equal to 75% denatured polypeptides. In some embodiments, the one or polypeptides in the composition comprises less than or equal to 75%, less than or equal to 70%, less than or equal to 65%, less than or equal to 60%, less than or equal to 55%, or less than or equal to 50% denatured polypeptides. Combinations of these ranges are possible (e.g., greater than or equal to 50% and less than or equal to 75%). Other ranges are also possible.

In some embodiments, the composition comprises some of the plurality of particles. In some embodiments, the plurality of particles may be associated with one or more denatured polypeptides. In some embodiments, the plurality of particles can be coupled to at least some of the polypeptides. In some embodiments, the plurality of particles can be bonded to at least some of the polypeptides.

Without wishing to be bound by any particular theory, the composition may indicate that the mixture was separated from the fluid by the denaturation of one or more polypeptides. Upon denaturation, hydrophobic portions of the denatured polypeptides, that may not be fully exposed prior to denaturation, may be exposed and associate with some of the plurality of particles (e.g., plastic particles). Accordingly, in some embodiments, the composition comprises the denatured polypeptides associated with (e.g., adsorbed onto, bonded to) some of the plurality of particles. The presence of denatured polypeptides associated with some of the plurality of particles may indicate that separation of the mixture from the fluid was carried out using the methods described herein.

In some embodiments, some of the plurality of particles are separated from the fluid in a vessel. For example, in FIG. 1B, agglomerate 125 is separated from fluid 105 in vessel 120. The vessel may have any of a myriad of forms including but not limited to a bioreactor, a flask, a petri dish, a consumable container (e.g., a centrifuge tube), a vial, and/or a container. In some embodiments, the vessel comprises one or more inlets configured to introduce fluids, chemical compounds (e.g., salts, buffers, etc.), and/or polypeptides (e.g., the polymer-degrading enzyme, the sacrificial polypeptide, coenzymes). In some embodiments, the vessel comprises one or more outlets configured to output at least some of the contents of the vessel. In some embodiments, the vessel is configured to maintain and/or controllably alter any of a variety of reaction conditions including temperature, pH, and/or pressure. As an example, the vessel may comprise a thermocouple to monitor the temperature within the vessel and/or a heat source to vary the temperature within the vessel. As another example, the vessel may comprise a sensor capable of determining the pH of the contents within the vessel and a mechanism to dose a chemical compound (e.g., sodium hydroxide) appropriately to maintain and/or alter the pH of the contents of the vessel.

In some embodiments, the one or more polypeptides comprises the polymer-degrading enzyme. In some embodiments, the polymer-degrading enzyme is a thermostable and/or thermophilic enzyme. In some embodiments, the polymer-degrading enzyme comprises a hydrolase, an esterase, a protease (e.g., a serine protease), a cutinase, a lipase, an oxidase, a peroxidase, and/or an amidase.

Examples of such polymer-degrading enzymes that are useful in methods and compositions provided herein are described in the following US, foreign, and international patents and patent application publications which are incorporated herein by reference in their entirety for all purposes: Japanese Patent No. 5,850,342 entitled “A novel esterase derived from twig leaf compost;” U.S. Pat. No. 11,414,651 entitled “Esterases and uses thereof;” Chinese Patent No. 113584057 entitled “ICCG expression element, expression vector, Bacillus subtilis recombinant strain and method for degrading PET or monomer thereof;” Chinese Patent No. 113684196 entitled “Purification method of high-temperature-resistant polyethylene terephthalate hydrolase;” U.S. Pat. No. 10,590,401 entitled “Esterases and uses thereof;” U.S. Pat. No. 11,535,832 entitled “Esterases and uses thereof;” U.S. Pat. No. 11,692,181 entitled “Esterases and uses thereof;” U.S. Pat. No. 10,584,320 entitled “Esterases and uses thereof;” U.S. Pat. No. 11,072,784 entitled “Esterases and uses thereof;” U.S. Pat. No. 6,995,005 entitled “DNA Sequences Coding for Ester-Group-Cleaving Enzymes;” Chinese Patent No. 101168735 entitled “High-temperature cutinase and gene order thereof;” U.S. application Ser. No. 13/517,331 entitled “Detergent Compositions Containing Thermobifida Fusca Lipase and Methods of use Thereof;” U.S. Pat. No. 11,773,383 entitled “Methods for Promoting Extracellular Expression of Proteins in Bacillus Subtilis Using a Cutinase;” U.S. patent application Ser. No. 14/237,846 entitled “Compositions and Methods Comprising a Lipolytic Enzyme Variant;” U.S. patent application Ser. No. 14/366,165 entitled “Compositions and Methods Comprising a Lipolytic Enzyme Variant;” International Application No. PCT/EP2021/079783 entitled “Novel esterases and their use;” EP3517608 entitled “New Polypeptides Having a Polyester Degrading Activity and Uses Thereof;” U.S. patent application Ser. No. 17/291,291 entitled “Method for the Enzymatic Degradation of Polyethylene Terephthalate;” U.S. patent application Ser. No. 17/625,783 entitled “Esterases And Uses Thereof;” International Application No. PCT/US2023/062092 entitled “Leaf-Branch Compost Cutinase Mutants;” U.S. Pat. No. 6,960,459 entitled “Fungal cutinase for use in the processing of textiles;” U.S. Pat. No. 9,476,072 entitled “Cutinase variants and polynucleotides encoding same;” U.S. Pat. No. 7,943,336 entitled “Cutinase for detoxification of feed products;” and U.S. Pat. No. 9,951,299 entitled “Cutinase variants and polynucleotides encoding same.”

Further examples of polymer-degrading enzymes that are useful in methods and compositions provided herein are described in the following literary publications which are incorporated herein by reference in their entirety for all purposes: Sulaiman S, Yamato S, Kanaya E, Kim J J, Koga Y, Takano K, Kanaya S. Isolation of a novel cutinase homolog with polyethylene terephthalate-degrading activity from leaf-branch compost by using a metagenomic approach. Appl Environ Microbiol. 2012 March;78 (5): 1556-62. doi: 10.1128/AEM.06725-11. Epub 2011 Dec. 22. PMID: 22194294; PMCID: PMC3294458; Tournier, V., Topham, C. M., Gilles, A. et al. An engineered PET depolymerase to break down and recycle plastic bottles. Nature 580, 216-219 (2020). doi.org/10.1038/s41586-020-2149-4; Then J, Wei R, Oeser T, Barth M, Belisario-Ferrari M R, Schmidt J, Zimmermann W. Ca2+ and Mg2+ binding site engineering increases the degradation of polyethylene terephthalate films by polyester hydrolases from Thermobifida fusca. Biotechnol J. 2015 April; 10 (4): 592-8. doi: 10.1002/biot.201400620. Epub 2015 Jan. 19. PMID: 25545638; Sonnendecker C, Oeser J, Richter P K, Hille P, Zhao Z, Fischer C, Lippold H, Blázquez-Sánchez P, Engelberger F, Ramírez-Sarmiento C A, Oeser T, Lihanova Y, Frank R, Jahnke H G, Billig S, Abel B, Sträter N, Matysik J, Zimmermann W. Low Carbon Footprint Recycling of Post-Consumer PET Plastic with a Metagenomic Polyester Hydrolase. ChemSusChem. 2022 May 6;15 (9): c202101062. doi: 10.1002/cssc.202101062. Epub 2022 Feb. 10. PMID: 34129279; PMCID: PMC9303343; Pfaff L, Gao J, Li Z, Jäckering A, Weber G, Mican J, Chen Y, Dong W, Han X, Feiler C G, Ao Y F, Badenhorst C P S, Bednar D, Palm G J, Lammers M, Damborsky J, Strodel B, Liu W, Bornscheuer U T, Wei R. Multiple Substrate Binding Mode-Guided Engineering of a Thermophilic PET Hydrolase. ACS Catal. 2022 Aug. 5;12 (15): 9790-9800. doi: 10.1021/acscatal.2c02275. Epub 2022 Jul. 27. PMID: 35966606; PMCID: PMC9361285; Richter, P. K., Blázquez-Sánchez, P., Zhao, Z. et al. Structure and function of the metagenomic plastic-degrading polyester hydrolase PHL7 bound to its product. Nat Commun 14, 1905 (2023). doi.org/10.1038/s41467-023-37415-x; Erickson, E., Gado, J. E., Avilán, L. et al. Sourcing thermotolerant poly (ethylene terephthalate) hydrolase scaffolds from natural diversity. Nat Commun 13, 7850 (2022). doi.org/10.1038/s41467-022-35237-x; Yoshida S, Hiraga K, Takchana T, Taniguchi I, Yamaji H, Macda Y, Toyohara K, Miyamoto K, Kimura Y, Oda K. A bacterium that degrades and assimilates poly (ethylene terephthalate). Science. 2016 Mar. 11;351 (6278): 1196-9. doi: 10.1126/science.aad6359. PMID: 26965627; Han X, Liu W, Huang J W, Ma J, Zheng Y, Ko T P, Xu L, Cheng Y S, Chen C C, Guo R T. Structural insight into catalytic mechanism of PET hydrolase. Nat Commun. 2017 Dec. 13;8 (1): 2106. doi: 10.1038/s41467-017-02255-z. PMID: 29235460; PMCID: PMC5727383; Cui H, Eltoukhy L, Zhang L, Markel U, Jaeger K E, Davari M D, Schwaneberg U. Less Unfavorable Salt Bridges on the Enzyme Surface Result in More Organic Cosolvent Resistance. Angew Chem Int Ed Engl. 2021 May 10;60 (20): 11448-11456. doi: 10.1002/anic.202101642. Epub 2021 Apr. 7. PMID: 33687787; PMCID: PMC8252522; Son J, Kalafatovic D, Kumar M, Yoo B, Cornejo M A, Contel M, Ulijn R V. Customizing Morphology, Size, and Response Kinetics of Matrix Metalloproteinase-Responsive Nanostructures by Systematic Peptide Design. ACS Nano. 2019 Feb. 26;13 (2): 1555-1562. doi: 10.1021/acsnano.8b07401. Epub 2019 Jan. 30. PMID: 30689363; PMCID: PMC6475088; Lu H, Diaz D J, Czarnecki N J, Zhu C, Kim W, Shroff R, Acosta D J, Alexander B R, Cole H O, Zhang Y, Lynd N A, Ellington A D, Alper H S. Machine learning-aided engineering of hydrolases for PET depolymerization. Nature. 2022 April;604 (7907): 662-667. doi: 10.1038/s41586-022-04599-z. Epub 2022 Apr. 27. PMID: 35478237; Bell, E. L., Smithson, R., Kilbride, S. et al. Directed evolution of an efficient and thermostable PET depolymerase. Nat Catal 5, 673-681 (2022). doi.org/10.1038/s41929-022-00821-3; Zhenkun Shi, Rui Dong, Qianqian Yuan, Zhitao Mao, Ruoyu Wang, Haoran Li, Xiaoping Liao, Hongwu Ma. Enzyme Commission Number Prediction and Benchmarking with Hierarchical Dual-core Multitask Learning Framework. Rescarch. 2023; 6:0153.DOI: 10.34133/rescarch.0153; Danso D, Schmeisser C, Chow J, Zimmermann W, Wei R, Leggewie C, Li X, Hazen T, Streit W R. New Insights into the Function and Global Distribution of Polyethylene Terephthalate (PET)-Degrading Bacteria and Enzymes in Marine and Terrestrial Metagenomes. Appl Environ Microbiol. 2018 Apr. 2;84 (8): c02773-17. doi: 10.1128/AEM.02773-17. PMID: 29427431; PMCID: PMC5881046; Blázquez-Sánchez P, Engelberger F, Cifuentes-Anticevic J, Sonnendecker C, Griñón A, Reyes J, Dícz B, Guixć V, Richter P K, Zimmermann W, Ramírez-Sarmiento C A. Antarctic Polyester Hydrolases Degrade Aliphatic and Aromatic Polyesters at Moderate Temperatures. Appl Environ Microbiol. 2022 Jan. 11;88 (1): c0184221. doi: 10.1128/AEM.01842-21. Epub 2021 Oct. 27. PMID: 34705547; PMCID: PMC8752145; Chen X, Liu M, Zhang P, Leung SSY, Xia J. Membrane-Permeable Antibacterial Enzyme against Multidrug-Resistant Acinetobacter baumannii. ACS Infect Dis. 2021 Aug. 13;7 (8): 2192-2204. doi: 10.1021/acsinfecdis. 1c00222. Epub 2021 Jul. 7. PMID: 34232613; Avilan L, Lichtenstein B R, König G, Zahn M, Allen M D, Oliveira L, Clark M, Bemmer V, Graham R, Austin H P, Dominick G, Johnson C W, Beckham G T, McGechan J E, Pickford A R. Concentration-Dependent Inhibition of Mesophilic PETases on Poly (ethylene terephthalate) Can Be Eliminated by Enzyme Engineering. ChemSusChem. 2023 Apr. 21;16 (8): c202202277. doi: 10.1002/cssc.202202277. Epub 2023 Mar. 23. PMID: 36811288; Meyer Cifuentes I E, Wu P, Zhao Y, Liu W, Neumann-Schaal M, Pfaff L, Barys J, Li Z, Gao J, Han X, Bornscheuer U T, Wei R, Öztürk B. Molecular and Biochemical Differences of the Tandem and Cold-Adapted PET Hydrolases Ple628 and Ple629, Isolated From a Marine Microbial Consortium. Front Bioeng Biotechnol. 2022 Jul. 21; 10:930140. doi: 10.3389/fbioc.2022.930140. PMID: 35935485; PMCID: PMC9350882; Inglis G D, Yanke L J, Selinger L B. Cutinolytic esterase activity of bacteria isolated from mixed-plant compost and characterization of a cutinase gene from Pseudomonas pseudoalcaligenes. Can J Microbiol. 2011 November;57 (11): 902-13. doi: 10.1139/w11-083. Epub 2011 Oct. 26. PMID: 22029433; Haernvall K, Zitzenbacher S, Wallig K, Yamamoto M, Schick M B, Ribitsch D, Gucbitz G M. Hydrolysis of Ionic Phthalic Acid Based Polyesters by Wastewater Microorganisms and Their Enzymes. Environ Sci Technol. 2017 Apr. 18;51 (8): 4596-4605. doi: 10.1021/acs.cst.7b00062. Epub 2017 Apr. 7. PMID: 28345898; Bollinger A, Thies S, Knieps-Grünhagen E, Gertzen C, Kobus S, Höppner A, Ferrer M, Gohlke H, Smits SHJ, Jaeger K E. A Novel Polyester Hydrolase From the Marine Bacterium Pseudomonas aestusnigri-Structural and Functional Insights. Front Microbiol. 2020 Fcb 13; 11:114. doi: 10.3389/fmich.2020.00114. PMID: 32117139; PMCID: PMC7031157; Macromolecules 2009, 42, 14, 5128-5138, Publication Date: Jul. 2, 2009, doi.org/10.1021/ma9005318; Wallace M, Green C R, Roberts L S, Lee Y M, McCarville J L, Sanchez-Gurmaches J, Meurs N, Gengatharan J M, Hover J D, Phillips S A, Ciaraldi T P, Guertin D A, Cabrales P, Ayres J S, Nomura D K, Loomba R, Metallo C M. Enzyme promiscuity drives branched-chain fatty acid synthesis in adipose tissues. Nat Chem Biol. 2018 November; 14 (11): 1021-1031. doi: 10.1038/s41589-018-0132-2. Epub 2018 Oct. 16. PMID: 30327559; PMCID: PMC6245668; Eiamthong B, Mecsawat P, Wongsatit T, Jitdec J, Sangsri R, Patchsung M, Aphicho K, Suraritdechachai S, Huguenin-Dezot N, Tang S, Suginta W, Paosawatyanyong B, Babu M M, Chin J W, Pakotiprapha D, Bhanthumnavin W, Uttamapinant C. Discovery and Genetic Code Expansion of a Polyethylene Terephthalate (PET) Hydrolase from the Human Saliva Metagenome for the Degradation and Bio-Functionalization of PET. Angew Chem Int Ed Engl. 2022 Scp 12;61 (37): c202203061. doi: 10.1002/anic.202203061. Epub 2022 Jun. 21. PMID: 35656865; PMCID: PMC7613822; Sagong H Y, Son H F, Sco H, Hong H, Lec D, Kim K J. Implications for the PET decomposition mechanism through similarity and dissimilarity between PETases from Rhizobacter gummiphilus and Ideonella sakaiensis. J Hazard Mater. 2021 Aug. 15; 416:126075. doi: 10.1016/j.jhazmat.2021.126075. Epub 2021 May 11. PMID: 34492896; Erickson E, Gado J E, Avilan L, Bratti F, Brizendine R K, Cox P A, Gill R, Graham R, Kim D J, König G, Michener W E, Poudel S, Ramirez K J, Shakespeare T J, Zahn M, Boyd E S, Payne C M, DuBois J L, Pickford A R, Beckham G T, McGechan J E. Sourcing thermotolerant poly (ethylene terephthalate) hydrolase scaffolds from natural diversity. Nat Commun. 2022 Dec. 21;13 (1): 7850. doi: 10.1038/s41467-022-35237-x. PMID: 36543766; PMCID: PMC9772341; Shirke A N, White C, Englaender J A, Zwarycz A, Butterfoss G L, Linhardt R J, Gross R A. Stabilizing Leaf and Branch Compost Cutinase (LCC) with Glycosylation: Mechanism and Effect on PET Hydrolysis. Biochemistry. 2018 Feb. 20;57 (7): 1190-1200. doi: 10.1021/acs.biochem.7b01189. Epub 2018 Jan. 30. PMID: 29328676; Xi X, Ni K, Hao H, Shang Y, Zhao B, Qian Z. Secretory expression in Bacillus subtilis and biochemical characterization of a highly thermostable polyethylene terephthalate hydrolase from bacterium HR29. Enzyme Microb Technol. 2021 February; 143:109715. doi: 10.1016/j.enzmictec.2020.109715. Epub 2020 Nov. 18. PMID: 33375975; Dresler K, van den Heuvel J, Müller R J, Deckwer W D. Production of a recombinant polyester-cleaving hydrolase from Thermobifida fusca in Escherichia coli. Bioprocess Biosyst Eng. 2006 August;29 (3): 169-83. doi: 10.1007/s00449-006-0069-9. Epub 2006 June 13. PMID: 16770590; PMCID: PMC1705536; Müller, R. J., Schrader, H., Profe, J., Dresler, K., & Deckwer, W. D. (2005). Enzymatic Degradation of Poly (ethylene terephthalatc): Rapid Hydrolyse using a Hydrolase fromT. fusca. Macromolecular Rapid Communications, 26 (17), 1400-1405. Kleeberg I, Welzel K, Vandenheuvel J, Müller R J, Deckwer W D. Characterization of a new extracellular hydrolase from Thermobifida fusca degrading aliphatic-aromatic copolyesters. Biomacromolecules. 2005 January-Feb;6 (1): 262-70. doi: 10.1021/bm049582t. PMID: 15638529; Macromolecules 2011, 44, 12, 4632-4640, Publication Date: May 20, 2011,https://doi.org/10.1021/ma200949p; Furukawa M, Kawakami N, Tomizawa A, Miyamoto K. Efficient Degradation of Poly (ethylene terephthalate) with Thermobifida fusca Cutinase Exhibiting Improved Catalytic Activity Generated using Mutagenesis and Additive-based Approaches. Sci Rep. 2019 Nov. 5;9 (1): 16038. doi: 10.1038/s41598-019-52379-z. PMID: 31690819; PMCID: PMC6831586; Roth C, Wei R, Oeser T, Then J, Föllner C, Zimmermann W, Sträter N. Structural and functional studies on a thermostable polyethylene terephthalate degrading hydrolase from Thermobifida fusca. Appl Microbiol Biotechnol. 2014 Scp;98 (18): 7815-23. doi: 10.1007/s00253-014-5672-0. Epub 2014 Apr. 13. PMID: 24728714; Wei R, Oeser T, Schmidt J, Meier R, Barth M, Then J, Zimmermann W. Engineered bacterial polyester hydrolases efficiently degrade polyethylene terephthalate due to relieved product inhibition. Biotechnol Bioeng. 2016 August; 113 (8): 1658-65. doi: 10.1002/bit.25941. Epub 2016 Fcb 4. PMID: 26804057; Ribitsch D, Hromic A, Zitzenbacher S, Zartl B, Gamerith C, Pellis A, Jungbauer A, Łyskowski A, Steinkellner G, Gruber K, Tscheliessnig R, Herrero Acero E, Guebitz G M. Small cause, large effect: Structural characterization of cutinases from Thermobifida cellulosilytica. Biotechnol Bioeng. 2017 November; 114 (11): 2481-2488. doi: 10.1002/bit.26372. Epub 2017 Aug. 15. PMID: 28671263; Ribitsch D, Herrero Acero E, Greimel K, Dellacher A, Zitzenbacher S, Marold A, Rodriguez R D, Steinkellner G, Gruber K, Schwab H, et al. A New Esterase from Thermobifida halotolerans Hydrolyses Polyethylene Terephthalate (PET) and Polylactic Acid (PLA). Polymers. 2012; 4 (1): 617-629. doi.org/10.3390/polym4010617; Kitadokoro K, Kakara M, Matsui S, Osokoshi R, Thumarat U, Kawai F, Kamitani S. Structural insights into the unique polylactate-degrading mechanism of Thermobifida alba cutinase. FEBS J. 2019 June;286 (11): 2087-2098. doi: 10.1111/febs.14781. Epub 2019 Feb. 28. PMID: 30761732; Miyakawa T, Mizushima H, Ohtsuka J, Oda M, Kawai F, Tanokura M. Structural basis for the Ca (2+)-enhanced thermostability and activity of PET-degrading cutinase-like enzyme from Saccharomonospora viridis AHK190. Appl Microbiol Biotechnol. 2015 May;99 (10): 4297-307. doi: 10.1007/s00253-014-6272-8. Epub 2014 Dec. 11. PMID: 25492421; Kawai F, Oda M, Tamashiro T, Waku T, Tanaka N, Yamamoto M, Mizushima H, Miyakawa T, Tanokura M. A novel Ca2-activated, thermostabilized polyesterase capable of hydrolyzing polyethylene terephthalate from Saccharomonospora viridis AHK190. Appl Microbiol Biotechnol. 2014 December;98 (24): 10053-64. doi: 10.1007/s00253-014-5860-y. Epub 2014 Jun. 15. PMID: 24929560; Oda M, Yamagami Y, Inaba S, Oida T, Yamamoto M, Kitajima S, Kawai F. Enzymatic hydrolysis of PET: functional roles of three Ca2+ ions bound to a cutinase-like enzyme, Cut 190*, and its engineering for improved activity. Appl Microbiol Biotechnol. 2018 Dcc; 102 (23): 10067-10077. doi: 10.1007/s00253-018-9374-x. Epub 2018 Sep. 24. PMID: 30250976; Jabloune R, Khalil M, Ben Moussa I E, Simao-Beaunoir A M, Lerat S, Brzezinski R, Beaulieu C. Enzymatic Degradation of p-Nitrophenyl Esters, Polyethylene Terephthalate, Cutin, and Suberin by Sub1, a Suberinase Encoded by the Plant Pathogen Streptomyces scabies. Microbes Environ. 2020;35 (1): ME19086. doi: 10.1264/jsme2.ME19086. PMID: 32101840; PMCID: PMC7104285; Carr C M, Keller M B, Paul B, Schubert S W, Clausen KSR, Jensen K, Clarke D J, Westh P, Dobson ADW. Purification and biochemical characterization of SM14est, a PET-hydrolyzing enzyme from the marine sponge-derived Streptomyces sp. SM14. Front Microbiol. 2023 May 12; 14:1170880. doi: 10.3389/fmicb.2023.1170880. PMID: 37250061; PMCID: PMC10213408; Tiong E, Koo Y S, Bi J, Koduru L, Koh W, Lim Y H, Wong F T. Expression and engineering of PET-degrading enzymes from Microbispora, Nonomuraca, and Micromonospora. Appl Environ Microbiol. 2023 Nov. 29;89 (11): e0063223. doi: 10.1128/aem.00632-23. Epub 2023 Nov. 9. PMID: 37943056; PMCID: PMC10686063; Ribitsch D, Heumann S, Trotscha E, Herrero Acero E, Greimel K, Leber R, Birner-Gruenberger R, Deller S, Eiteljoerg I, Remler P, Weber T, Siegert P, Maurer K H, Donelli I, Freddi G, Schwab H, Guebitz G M. Hydrolysis of polyethyleneterephthalate by p-nitrobenzylesterase from Bacillus subtilis. Biotechnol Prog. 2011 July;27 (4): 951-60. doi: 10.1002/btpr.610. Epub 2011 May 13. PMID: 21574267; Perz V, Zumstein M T, Sander M, Zitzenbacher S, Ribitsch D, Guebitz G M. Biomimetic Approach to Enhance Enzymatic Hydrolysis of the Synthetic Polyester Poly (1,4-butylene adipate): Fusing Binding Modules to Esterases. Biomacromolecules. 2015 Dec. 14;16 (12): 3889-96. doi: 10.1021/acs.biomac.5b01219. Epub 2015 Nov. 24. PMID: 26566664; Distaso M A, Chernikova T N, Bargicla R, Coscolín C, Stogios P, Gonzalez-Alfonso J L, Lemak S, Khusnutdinova A N, Plou F J, Evdokimova E, Savchenko A, Lunev E A, Yakimov M M, Golyshina O V, Ferrer M, Yakunin A F, Golyshin P N. Thermophilic Carboxylesterases from Hydrothermal Vents of the Volcanic Island of Ischia Active on Synthetic and Biobased Polymers and Mycotoxins. Appl Environ Microbiol. 2023 Feb. 28;89 (2): c0170422. doi: 10.1128/aem.01704-22. Epub 2023 Jan. 31. PMID: 36719236; PMCID: PMC9972953; Konstantinos Makryniotis, Efstratios Nikolaivits, Christina Gkountela, Stamatina Vouyiouka, Evangelos Topakas, Discovery of a polyesterase from Deinococcus maricopensis and comparison to the benchmark LCCICCG suggests high potential for semi-crystalline post-consumer PET degradation, Journal of Hazardous Materials, Volume 455, 2023, 131574, ISSN 0304-3894, doi.org/10.1016/j.jhazmat.2023.131574; ACS Catal. 2021, 11, 14, 8550-8564, Publication Date: Jun. 29, 2021, doi.org/10.1021/acscatal. 1c01204; Carnicl, A., Valoni, É., Nicomedes, J., Gomes, A. D., & Castro, A. M. (2017). Lipase from Candida antarctica (CALB) and cutinase from Humicola insolens act synergistically for PET hydrolysis to terephthalic acid. Process Biochemistry, 59, 84-90; Brackmann R, de Oliveira Veloso C, de Castro A M, Langone MAP. Enzymatic post-consumer poly (ethylene terephthalate) (PET) depolymerization using commercial enzymes. 3 Biotech. 2023 May;13 (5): 135. doi: 10.1007/s13205-023-03555-6. Epub 2023 Apr. 25. PMID: 37124991; PMCID: PMC10130296; Silva, C. M., Carneiro, F., O'Neill, A., Fonseca, L. P., Cabral, J. S., Guebitz, G., & Cavaco-Paulo, A. (2005). Cutinase-a new tool for biomodification of synthetic fibers. Journal of Polymer Science Part A: Polymer Chemistry, 43 (11), 2448-2450; Dimarogona, M., Nikolaivits, E., Kanelli, M., Christakopoulos, P., Sandgren, M., & Topakas, E. (2015). Structural and functional studies of a Fusarium oxysporum cutinase with polyethylene terephthalate modification potential. Biochimica et Biophysica Acta (BBA)-General Subjects, 1850 (11), 2308-2317; Brueckner, T., Eberl, A., Heumann, S., Rabe, M., & Gucbitz, G. M. (2008). Enzymatic and chemical hydrolysis of poly (ethylene terephthalate) fabrics. Journal of Polymer Science Part A: Polymer Chemistry, 46 (19), 6435-6443; Eberl, A., Heumann, S., Brückner, T., Araujo, R., Cavaco-Paulo, A., Kaufmann, F., . . . & Guebitz, G. M. (2009). Enzymatic surface hydrolysis of poly (cthylene terephthalate) and bis (benzoyloxyethyl) terephthalate by lipase and cutinase in the presence of surface active molecules. Journal of biotechnology, 143 (3), 207-212; Vázquez-Alcántara, L., Oliart-Ros, R. M., García-Bórquez, A., & Peña-Montes, C. (2021). Expression of a cutinase of Moniliophthora roreri with polyester and PET-plastic residues degradation activity. Microbiology Spectrum, 9 (3), e00976-21; Robles-Martín, A., Amigot-Sánchez, R., Fernandez-Lopez, L., Gonzalez-Alfonso, J. L., Roda, S., Alcolea-Rodriguez, V., . . . & Guallar, V. (2023). Sub-micro- and nano-sized polyethylene terephthalate deconstruction with engineered protein nanopores. Nature Catalysis, 1-12; Sevilla M E, Garcia M D, Perez-Castillo Y, Armijos-Jaramillo V, Casado S, Vizucte K, Debut A, Cerda-Mejía L. Degradation of PET Bottles by an Engineered Ideonella sakaiensis PETasc. Polymers. 2023; 15 (7): 1779. doi.org/10.3390/polym15071779; Edwards S, León-Zayas R, Ditter R, Laster H, Shechan G, Anderson O, Beattie T, Mellies J L. Microbial Consortia and Mixed Plastic Waste: Pangenomic Analysis Reveals Potential for Degradation of Multiple Plastic Types via Previously Identified PET Degrading Bacteria. International Journal of Molecular Sciences. 2022; 23 (10): 5612.doi.org/10.3390/ijms23105612; Ho, N. H. E., Effendi, S. S. W., Ting, W. W., Yi, Y. C., Yu, J. Y., Chang, J. S., & Ng, I. S. (2023). Heterologous expression and characterization of Aquabacterium parvum lipase, a close relative of Ideonella sakaiensis PETase in Escherichia coli. Biochemical Engineering Journal, 197, 108985; Qi, X., Ji, M., Yin, C.-F., Zhou, N.-Y. & Liu, Y. (2023) Glacier as a source of novel polyethylene terephthalate hydrolases. Environmental Microbiology, 25 (12), 2822-2833. Available from: doi.org/10.1111/1462-2920.16516; Chen S, Tong X, Woodard R W, Du G, Wu J, Chen J. Identification and characterization of bacterial cutinase. J Biol Chem. 2008 Sep. 19;283 (38): 25854-62. doi: 10.1074/jbc.M800848200. Epub 2008 Jul. 24. PMID: 18658138; PMCID: PMC3258855; Su L, Woodard R W, Chen J, Wu J. Extracellular location of Thermobifida fusca cutinase expressed in Escherichia coli BL21 (DE3) without mediation of a signal peptide. Appl Environ Microbiol. 2013 July;79 (14): 4192-8. doi: 10.1128/AEM.00239-13. Epub 2013 Apr. 19. PMID: 23603671; PMCID: PMC3697513; Lykidis A, Mavromatis K, Ivanova N, Anderson I, Land M, DiBartolo G, Martinez M, Lapidus A, Lucas S, Copeland A, Richardson P, Wilson D B, Kyrpides N. Genome sequence and analysis of the soil cellulolytic actinomycete Thermobifida fusca Y X. J Bacteriol. 2007 March; 189 (6): 2477-86. doi: 10.1128/J B.01899-06. Epub 2007 Jan. 5. PMID: 17209016; PMCID: PMC1899369; Hegde K, Vecranki V D. Production optimization and characterization of recombinant cutinases from Thermobifida fusca sp. NRRL B-8184. Appl Biochem Biotechnol. 2013 June; 170 (3): 654-75. doi: 10.1007/s12010-013-0219-x. Epub 2013 Apr. 19. PMID: 23604968; Wei R, Oeser T, Then J, Kühn N, Barth M, Schmidt J, Zimmermann W. Functional characterization and structural modeling of synthetic polyester-degrading hydrolases from Thermomonospora curvata. AMB Express. 2014 Jun. 3; 4:44. doi: 10.1186/s13568-014-0044-9. PMID: 25405080; PMCID: PMC4231364; Hu X, Thumarat U, Zhang X, Tang M, Kawai F. Diversity of polyester-degrading bacteria in compost and molecular analysis of a thermoactive esterase from Thermobifida alba AHK119. Appl Microbiol Biotechnol. 2010 June;87 (2): 771-9. doi: 10.1007/s00253-010-2555-x. PMID: 20393707; Almeida E L, Carrillo Rincón A F, Jackson S A, Dobson ADW. In silico Screening and Heterologous Expression of a Polyethylene Terephthalate Hydrolase (PETase)-Like Enzyme (SM14est) With Polycaprolactone (PCL)-Degrading Activity, From the Marine Sponge-Derived Strain Streptomyces sp. SM14. Front Microbiol. 2019 Oct. 1; 10:2187. doi: 10.3389/fmich.2019.02187. PMID: 31632361; PMCID: PMC6779837; Zhang, H., Dierkes, R. F., Perez-Garcia, P., Costanzi, E., Dittrich, J., Cea, P. A., Gurschke, M., Applegate, V., Partus, K., Schmeisser, C., Pfleger, C., Gohlke, H., Smits, S. H. J., Chow, J. and Streit, W. R. (2024), The metagenome-derived esterase PET40 is highly promiscuous and hydrolyses polyethylene terephthalate (PET). FEBS J, 291:70-91. doi.org/10.1111/febs. 16924; Zhang H, Perez-Garcia P, Dierkes R F, Applegate V, Schumacher J, Chibani C M, Sternagel S, Preuss L, Weigert S, Schmeisser C, Danso D, Pleiss J, Almeida A, Höcker B, Hallam S J, Schmitz R A, Smits SHJ, Chow J, Streit W R. The Bacteroidetes Aequorivita sp. and Kaistella jeonii Produce Promiscuous Esterases With PET-Hydrolyzing Activity. Front Microbiol. 2022 Jan. 5; 12:803896. doi: 10.3389/fmich.2021.803896. PMID: 35069509; PMCID: PMC8767016; and Perez-Garcia, P., Chow, J., Costanzi, E. et al. An archacal lid-containing feruloyl esterase degrades polyethylene terephthalate. Commun Chem 6, 193 (2023). https://doi.org/10.1038/s42004-023-00998-2.

Further examples of such polymer-degrading enzymes that are useful in methods and compositions provided herein are listed Table 1. In some embodiments, a polymer-degrading enzyme useful in methods and compositions provided herein has an amino acid sequence set forth in any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38. In some embodiments, the polymer-degrading enzyme is a variant of any one of the foregoing enzymes in which the variant has an insertion, deletion, or substitution of up to 1, 2, 3, 4, 5, 6, 7, 8, 9, 10, 11, 12, 13, 14, 15, 16, 17, 18, 19, or 20 amino acids compared with an amino acid sequence set forth in any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38. In some embodiments, the polymer-degrading enzyme is a variant of any one of the foregoing enzymes, in which the variant has at least 85%, 86%, 87%, 88%, 89%, 90%, 91%, 92%, 93%, 94%, 95%, 96%, 97%, 98%, or 99% identity compared to an amino acid sequence set forth in any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38.

TABLE 1
Examples of Polymer-degrading enzymes

| Accession | | | | | | | |
|---|---|---|---|---|---|---|---|
| Number | | | | | | | |
| (UniProt or | SEQ ID | | Common Name | | | EC | ESTHER |
| GenBank) | NO: | Sequence | (if any) | Pfam | InterPro | number | family |
| A0A075B5G4 | 1 | QLGAIENGLES | HiC | PF01083; | IPR029058; | 3.1.1.74 | Cutinase |
| | | GSANACPDAIL | | | IPR000675; | | |
| | | IFARGSTEPGN | | | IPR043580; | | |
| | | MGITVGPALA | | | IPR043579; | | |
| | | NGLESHIRNIW | | | IPR011150; | | |
| | | IQGVGGPYDA | | | | | |
| | | ALATNFLPRG | | | | | |
| | | TSQANIDEGK | | | | | |
| | | RLFALANQKC | | | | | |
| | | PNTPVVAGGY | | | | | |
| | | SQGAALIAAA | | | | | |
| | | VSELSGAVKE | | | | | |
| | | QVKGVALFGY | | | | | |
| | | TQNLQNRGGI | | | | | |
| | | PNYPRERTKV | | | | | |
| | | FCNVGDAVCT | | | | | |
| | | GTLIITPAHLS | | | | | |
| | | YTIEARGEAA | | | | | |
| | | RFLRDRIRA | | | | | |
| A0A0G3BI90 | 2 | MPPDCVLPRR | PET12; PbPL | PF01738; | IPR029058; | | Polyesterase- |
| | | LAAAALLASA | | | IPR002925; | | lipase- |
| | | TLVPLSAAAQ | | | | | cutinase |
| | | TNPYQRGPDP | | | | | |
| | | TTRDLEDSRGP | | | | | |
| | | FRYASTNVRSP | | | | | |
| | | SGYGAGTIYY | | | | | |
| | | PTDVSGSVGA | | | | | |
| | | VAVVPGYLAR | | | | | |
| | | QSSIRWWGPR | | | | | |
| | | LASHGFVVITL | | | | | |
| | | DTRSTSDQPAS | | | | | |
| | | RSAQQMAALR | | | | | |
| | | QVVALSETRSS | | | | | |
| | | PIYGKVDPNRL | | | | | |
| | | AVMGWSMGG | | | | | |
| | | GGTLISARDNP | | | | | |
| | | SLKAAVPFAP | | | | | |
| | | WHNTANFSGV | | | | | |
| | | QVPTLVIACEN | | | | | |
| | | DTVAPISRHAS | | | | | |
| | | SFYNSFSSSLA | | | | | |
| | | KAYLEINNGS | | | | | |
| | | HTCANTGNSN | | | | | |
| | | QALIGKYGVA | | | | | |
| | | WIKRFVDNDT | | | | | |
| | | RYSPFLCGAPH | | | | | |
| | | QADLRSSRLSE | | | | | |
| | | YRESCPY | | | | | |
| A0A0K8P6T7 | 3 | MNFPRASRLM | isPETase | PF01738; | IPR029058; | 3.1.1.101 | Polyesterase- |
| | | QAAVLGGLM | | | IPR002925; | | lipase- |
| | | AVSAAATAQT | | | | | cutinase |
| | | NPYARGPNPT | | | | | |
| | | AASLEASAGPF | | | | | |
| | | TVRSFTVSRPS | | | | | |
| | | GYGAGTVYYP | | | | | |
| | | TNAGGTVGAI | | | | | |
| | | AIVPGYTARQS | | | | | |
| | | SIKWWGPRLA | | | | | |
| | | SHGFVVITIDT | | | | | |
| | | NSTLDQPSSRS | | | | | |
| | | SQQMAALRQV | | | | | |
| | | ASLNGTSSSPI | | | | | |
| | | YGKVDTARM | | | | | |
| | | GVMGWSMGG | | | | | |
| | | GGSLISAANNP | | | | | |
| | | SLKAAAPQAP | | | | | |
| | | WDSSTNFSSV | | | | | |
| | | TVPTLIFACEN | | | | | |
| | | DSIAPVNSSAL | | | | | |
| | | PIYDSMSRNA | | | | | |
| | | KQFLEINGGSH | | | | | |
| | | SCANSGNSNQ | | | | | |
| | | ALIGKKGVAW | | | | | |
| | | MKRFMDNDT | | | | | |
| | | RYSTFACENP | | | | | |
| | | NSTRVSDFRT | | | | | |
| | | ANCS | | | | | |
| A0A1F4JXW8 | 4 | MAVGSMLLS | BurPL | PF01738; | IPR029058; | | Polyesterase- |
| | | MAAQAQVVV | | | IPR002925; | | lipase- |
| | | FEETFSTGLGK | | | | | cutinase |
| | | FTAAGSVVTSS | | | | | |
| | | GAARLDGCYG | | | | | |
| | | CTDGSITSTAIS | | | | | |
| | | TVDFTGLRLSF | | | | | |
| | | DRVTSGLDSG | | | | | |
| | | EAGIAEFSTNG | | | | | |
| | | STYTAVESIRT | | | | | |
| | | ASGRVTFNLPT | | | | | |
| | | SAENQSGLRL | | | | | |
| | | RFRINASLSSE | | | | | |
| | | TYTVDNIRLEG | | | | | |
| | | TSGSGGGTTN | | | | | |
| | | PFEKGPDPTKT | | | | | |
| | | MLEASTGPFT | | | | | |
| | | YTTTTVSSTTA | | | | | |
| | | SGYRQGTIYHP | | | | | |
| | | TNVTGPFAAV | | | | | |
| | | AVVPGYLASQ | | | | | |
| | | SSINWWGPRL | | | | | |
| | | ASHGFVVITID | | | | | |
| | | TNSTSDQPPSR | | | | | |
| | | ATQLMAALNQ | | | | | |
| | | LKTFSNTSSHP | | | | | |
| | | IYRKVDPNRL | | | | | |
| | | GVMGWSMGG | | | | | |
| | | GGTLIAARDN | | | | | |
| | | PTLKAAIPFAP | | | | | |
| | | WNSSTNFSTV | | | | | |
| | | SVPTLIIACESD | | | | | |
| | | STAPVNSHASP | | | | | |
| | | FYNSLPSTTKK | | | | | |
| | | AYLEMNNGSH | | | | | |
| | | SCANSGNSNA | | | | | |
| | | GLIGKYGVSW | | | | | |
| | | MKRFMDNDT | | | | | |
| | | RFSPYLCGAPH | | | | | |
| | | QADLSLTAIDE | | | | | |
| | | YRENCPY | | | | | |
| A0A1H6AD45 | 5 | MPFNKKSVLA | PE-H | PF01738; | IPR029058; | | Polyesterase- |
| | | LCGAGALLFS | | | IPR002925; | | lipase- |
| | | MSALANNPAP | | | | | cutinase |
| | | TDPGDSGGGS | | | | | |
| | | AYQRGPDPSV | | | | | |
| | | SFLEADRGQY | | | | | |
| | | SVRSSRVSSLV | | | | | |
| | | SGFGGGTIYYP | | | | | |
| | | TGTTGTMGAV | | | | | |
| | | VVIPGFVSAES | | | | | |
| | | SIDWWGPKLA | | | | | |
| | | SYGFVVMTID | | | | | |
| | | TNTGFDQPPSR | | | | | |
| | | ARQINNALDY | | | | | |
| | | LVSQNSRSSSP | | | | | |
| | | VRGMIDTNRL | | | | | |
| | | GVIGWSMGGG | | | | | |
| | | GTLRVASEGRI | | | | | |
| | | KAAIPLAPWD | | | | | |
| | | TTSYYASRSQ | | | | | |
| | | APTLIFACESD | | | | | |
| | | VIAPVLQHASP | | | | | |
| | | FYNSLPSSIDK | | | | | |
| | | AFVEINGGSH | | | | | |
| | | YCGNGGSIYN | | | | | |
| | | DVLSRFGVSW | | | | | |
| | | MKLHLDEDSR | | | | | |
| | | YKQFLCGPNH | | | | | |
| | | TSDSQISDYRG | | | | | |
| | | NCPY | | | | | |
| A0A1W6L588 | 6 | MFGKLPFARA | RgPETase | PF01738; | IPR029058; | 3.1.1.101 | Polyesterase- |
| | | SLAVGALLLS | | | IPR002925; | | lipase- |
| | | AAAVAQTNPY | | | | | cutinase |
| | | QRGPDPTVSSL | | | | | |
| | | EATRGPFSTSS | | | | | |
| | | FTVSRPSGYG | | | | | |
| | | AGTVYYPTNA | | | | | |
| | | GGKVGAIAVV | | | | | |
| | | PGYTARQSSIN | | | | | |
| | | WWGPRLASH | | | | | |
| | | GFVVITIDTNS | | | | | |
| | | TLDQPSSRSSQ | | | | | |
| | | QMAALRQVVS | | | | | |
| | | LAGTSSSPIYN | | | | | |
| | | KVDTARLGV | | | | | |
| | | MGWSMGGGG | | | | | |
| | | SLISAKNNPSL | | | | | |
| | | RAAAPQAPWA | | | | | |
| | | QESFSSVTVPT | | | | | |
| | | LIVSCENDSIA | | | | | |
| | | PNSSHSFPFYN | | | | | |
| | | QMTRNKKAN | | | | | |
| | | LVINGGSHSCA | | | | | |
| | | NSGNSDAGLI | | | | | |
| | | GKYGVAWMK | | | | | |
| | | RFMDDDTRYS | | | | | |
| | | KFLCGAEHQA | | | | | |
| | | DLSKRAVEAY | | | | | |
| | | KENCPY | | | | | |
| A0A2H5Z9R5 | 7 | MQVVLGRVRS | BhrPETase | PF12695; | IPR029058; | | Polyesterase- |
| | | AGLLAALLAL | | | IPR029059; | | lipase- |
| | | AAWALVWAS | | | | | cutinase |
| | | PSAEAQSNPY | | | | | |
| | | QRGPNPTRSA | | | | | |
| | | LTTDGPFSVAT | | | | | |
| | | YSVSRLSVSGF | | | | | |
| | | GGGVIYYPTG | | | | | |
| | | TTLTFGGIAMS | | | | | |
| | | PGYTADASSL | | | | | |
| | | AWLGRRLASH | | | | | |
| | | GFVVIVINTNS | | | | | |
| | | RLDFPDSRASQ | | | | | |
| | | LSAALNYLRT | | | | | |
| | | SSPSAVRARLD | | | | | |
| | | ANRLAVAGHS | | | | | |
| | | MGGGATLRIS | | | | | |
| | | EQIPTLKAGVP | | | | | |
| | | LTPWHTDKTF | | | | | |
| | | NTPVPQLIVGA | | | | | |
| | | EADTVAPVSQ | | | | | |
| | | HAIPFYQNLPS | | | | | |
| | | TTPKVYVELD | | | | | |
| | | NATHFAPNSP | | | | | |
| | | NAAISVYTISW | | | | | |
| | | MKLWVDNDT | | | | | |
| | | RYRQFLCNVN | | | | | |
| | | DPALSDFRSN | | | | | |
| | | NRHCQ | | | | | |
| A0A5P3LVN0 | 8 | MAACTDIDVV | Sub1 | PF01083; | IPR029058; | | Cutinase |
| | | SARGTFEPGTL | | | IPR000675; | | |
| | | GFIVGDPVYA | | | | | |
| | | ALQKKVAGKS | | | | | |
| | | LSSYKVNYPA | | | | | |
| | | DLSPTSAAQG | | | | | |
| | | NADLVNHVRS | | | | | |
| | | QAASCPNQRF | | | | | |
| | | VLVGYSQGAN | | | | | |
| | | VVDNSIGISSA | | | | | |
| | | GAVVGSPIVA | | | | | |
| | | TLPAALEPRVS | | | | | |
| | | AVLLFGNPIRA | | | | | |
| | | IGKSVTGTYQS | | | | | |
| | | RTIDFCAAGDP | | | | | |
| | | VCENGGGDVG | | | | | |
| | | AHLGYRANAD | | | | | |
| | | AAAAFAATKI | | | | | |
| A5I055 | 9 | MKKNLNKIAT | del71Cbotu_ | | IPR029058; | | Bacterial_ |
| | | IILLVFSMTLT | EstA | | | | lip_ |
| | | NFSMIVRAAEP | | | | | Faml.5 |
| | | KAQGTQKVES | | | | | |
| | | STTKKEVKDA | | | | | |
| | | EETIKIPTLEDI | | | | | |
| | | DNLIDSAEEVK | | | | | |
| | | SEEDINKMPPL | | | | | |
| | | KFPVEFPEVNT | | | | | |
| | | RSIIGGNNYPI | | | | | |
| | | VLVHGFMGFG | | | | | |
| | | RDELLGYKYW | | | | | |
| | | GGVVDLQEKL | | | | | |
| | | NASGHETYTA | | | | | |
| | | TVGPVSSNWD | | | | | |
| | | RACELYAYIV | | | | | |
| | | GGTVDYGEAH | | | | | |
| | | AKKFKHNRYG | | | | | |
| | | RTYPGIYKNIS | | | | | |
| | | NENKIHLIGHS | | | | | |
| | | MGGQTIRTLT | | | | | |
| | | QLLSEGSEEEI | | | | | |
| | | NCGQENISPLF | | | | | |
| | | EGGKHWIHSV | | | | | |
| | | STISTPNDGTT | | | | | |
| | | LSDLMPAKDLI | | | | | |
| | | SYTFGVLGTIT | | | | | |
| | | GKNKLFSSIYD | | | | | |
| | | LKLDQWGLK | | | | | |
| | | KQNGESQRDY | | | | | |
| | | IERVLDSNIWN | | | | | |
| | | STKDIATYDLS | | | | | |
| | | TEGAQELNTW | | | | | |
| | | VKAQPDVYYF | | | | | |
| | | SWTTQATKESI | | | | | |
| | | LTGHSVAQIGP | | | | | |
| | | MNPIFYPTANL | | | | | |
| | | MGRYSRNQK | | | | | |
| | | DLPIIDKKWFP | | | | | |
| | | NDGVVNCISQ | | | | | |
| | | DGPKLGSNDV | | | | | |
| | | IEQYNGGVKIG | | | | | |
| | | QWNAMPRIIN | | | | | |
| | | TDHMDIVGTF | | | | | |
| | | GNVKDWYMD | | | | | |
| | | YASFLSNLSR | | | | | |
| C3RYL0 | 10 | MPITARNTLAS | PET2 | PF01738; | IPR029058; | | Polyesterase- |
| | | LLLASSALLLS | | | IPR002925; | | lipase- |
| | | GTAFAANPPG | | | | | cutinase |
| | | GDPDPGCQTD | | | | | |
| | | CNYQRGPDPT | | | | | |
| | | DAYLEAASGP | | | | | |
| | | YTVSTIRVSSL | | | | | |
| | | VPGFGGGTIH | | | | | |
| | | YPTNAGGGK | | | | | |
| | | MAGIVVIPGYL | | | | | |
| | | SFESSIEWWGP | | | | | |
| | | RLASHGFVVM | | | | | |
| | | TIDTNTIYDQP | | | | | |
| | | SQRRDQIEAAL | | | | | |
| | | QYLVNQSNSS | | | | | |
| | | SSPISGMVDSS | | | | | |
| | | RLAAVGWSM | | | | | |
| | | GGGGTLQLAA | | | | | |
| | | DGGIKAAIALA | | | | | |
| | | PWNSSINDFNR | | | | | |
| | | IQVPTLIFACQ | | | | | |
| | | LDALAPVALH | | | | | |
| | | ASPFYNRIPNT | | | | | |
| | | TPKAFFEMTG | | | | | |
| | | GDHWCANGG | | | | | |
| | | NIYSALLGKY | | | | | |
| | | GVSWMKLHL | | | | | |
| | | DQDTRYAPFL | | | | | |
| | | CGPNHAAQTL | | | | | |
| | | ISEYRGNCPY | | | | | |
| D1A2H1 | 11 | MKRTLKRALS | Teur0390 | PF03403; | IPR029058; | 3.1.1.3 | Polyesterase- |
| | | LLPAAALAAS | | | | | lipase- |
| | | ALVAASPAQA | | | | | cutinase |
| | | AANPYQRGPN | | | | | |
| | | PTEASITAARG | | | | | |
| | | PFNTAEITVSR | | | | | |
| | | LSVSGFGGGKI | | | | | |
| | | YYPTTTSEGTF | | | | | |
| | | GALAISPGFTA | | | | | |
| | | YWSSLEWLGH | | | | | |
| | | RLASQGFVVIG | | | | | |
| | | IETNTTLDQPD | | | | | |
| | | QRGQQLLAAL | | | | | |
| | | DYLTQRSAVR | | | | | |
| | | DRVDASRLAV | | | | | |
| | | AGHSMGGGGS | | | | | |
| | | LEAAKARTSL | | | | | |
| | | KAAIPLAPWN | | | | | |
| | | LDKTWPEVRT | | | | | |
| | | PTLIIGGELDA | | | | | |
| | | VAPVATHSIPF | | | | | |
| | | YNSLSNAPEK | | | | | |
| | | AYLELDNASH | | | | | |
| | | FFPNITNTQMA | | | | | |
| | | KYMIAWMKR | | | | | |
| | | FIDDDTRYTQF | | | | | |
| | | LCPPPSTGLLS | | | | | |
| | | DFSDARFTCP | | | | | |
| | | M | | | | | |
| D1A9G5 | 12 | MSLRKSFGLLS | Tour1278 | PF03403; | IPR029058; | 3.1.1.3 | Polyesterase- |
| | | ATAALVAGLV | | | | | lipase- |
| | | AAPPAQAAAN | | | | | cutinase |
| | | PYQRGPDPTES | | | | | |
| | | LLRAARGPFA | | | | | |
| | | VSEQSVSRLSV | | | | | |
| | | SGFGGGRIYYP | | | | | |
| | | TTTSQGTFGAI | | | | | |
| | | AISPGFTASWS | | | | | |
| | | SLAWLGPRLA | | | | | |
| | | SHGFVVIGIET | | | | | |
| | | NTRLDQPDSR | | | | | |
| | | GRQLLAALDY | | | | | |
| | | LTQRSSVRNR | | | | | |
| | | VDASRLAVAG | | | | | |
| | | HSMGGGGTLE | | | | | |
| | | AAKSRTSLKA | | | | | |
| | | AIPLAPWNLDK | | | | | |
| | | TWPEVRTPTLI | | | | | |
| | | IGGELDSIAPV | | | | | |
| | | ATHSIPFYNSL | | | | | |
| | | TNAREKAYLE | | | | | |
| | | LNNASHFFPQF | | | | | |
| | | SNDTMAKFMI | | | | | |
| | | SWMKRFIDDD | | | | | |
| | | TRYDQFLCPPP | | | | | |
| | | RAIGDISDYRD | | | | | |
| | | TCPHT | | | | | |
| D4Q9N1 | 13 | MSVTTPRREA | Est1 | PF12740; | IPR029058; | 3.1.1.101; | Polyesterase- |
| | | SLLSRAVAVA | | | IPR041127; | 3.1.1.74 | lipase- |
| | | AAAAATVALA | | | | | cutinase |
| | | APAQAANPYE | | | | | |
| | | RGPNPTESML | | | | | |
| | | EARSGPFSVSE | | | | | |
| | | ERASRLGADG | | | | | |
| | | FGGGTIYYPRE | | | | | |
| | | NNTYGALAISP | | | | | |
| | | GYTGTQSSIA | | | | | |
| | | WLGERIASHG | | | | | |
| | | FVVIAIDTNTT | | | | | |
| | | LDQPDSRARQ | | | | | |
| | | LNAALDYMLT | | | | | |
| | | DASSSVRNRID | | | | | |
| | | ASRLAVMGHS | | | | | |
| | | MGGGGTLRLA | | | | | |
| | | SQRPDLKAAIP | | | | | |
| | | LTPWHLNKSW | | | | | |
| | | RDITVPTLIIGA | | | | | |
| | | DLDTIAPVSSH | | | | | |
| | | SEPFYNSIPSST | | | | | |
| | | DKAYLELNNA | | | | | |
| | | THFAPNITNKT | | | | | |
| | | IGMYSVAWLK | | | | | |
| | | RFVDEDTRYT | | | | | |
| | | QFLCPGPRTGL | | | | | |
| | | LSDVDEYRST | | | | | |
| | | CPF | | | | | |
| D7R6G8 | 14 | MTHQIVTTQY | BsEstB | PF00135; | IPR029058; | 3.1.1.- | Carb_B_ |
| | | GKVKGTTENG | | | IPR002018; | | Bacteria |
| | | VHKWKGIPYA | | | IPR019826; | | |
| | | KPPVGQWRFK | | | IPR019819; | | |
| | | APEPPEVWED | | | IPR000997; | | |
| | | VLDATAYGPI | | | | | |
| | | CPQPSDLLSLS | | | | | |
| | | YTELPRQSEDC | | | | | |
| | | LYVNVFAPDT | | | | | |
| | | PSQNLPVMVW | | | | | |
| | | IHGGAFYLGA | | | | | |
| | | GSEPLYDGSK | | | | | |
| | | LAAQGEVIVV | | | | | |
| | | TLNYRLGPFGF | | | | | |
| | | LHLSSFNEAYS | | | | | |
| | | DNLGLLDQAA | | | | | |
| | | ALKWVRENIS | | | | | |
| | | AFGGDPDNVT | | | | | |
| | | VFGESAGGMS | | | | | |
| | | IAALLAMPAA | | | | | |
| | | KGLFQKAIME | | | | | |
| | | SGASRTMTKE | | | | | |
| | | QAASTSAAFL | | | | | |
| | | QVLGINEGQL | | | | | |
| | | DKLHTVSAED | | | | | |
| | | SLKAADQLRI | | | | | |
| | | AEKENIFQLFF | | | | | |
| | | QPALDPKTLPE | | | | | |
| | | EPEKAIAEGAA | | | | | |
| | | SGIPLLIGTTRD | | | | | |
| | | EGYLFFTPDSD | | | | | |
| | | VHSQETLDAA | | | | | |
| | | LEYLLGKPLA | | | | | |
| | | EKAADLYPRS | | | | | |
| | | LESQIHMMTD | | | | | |
| | | LLFWRPAVAY | | | | | |
| | | ASAQSHYAPV | | | | | |
| | | WMYRFDWHP | | | | | |
| | | KKPPYNKAFH | | | | | |
| | | ALELPFVFGNL | | | | | |
| | | DGLERMAKAE | | | | | |
| | | ITDEVKQLSHT | | | | | |
| | | IQSAWITFAKT | | | | | |
| | | GNPSTEAVNW | | | | | |
| | | PAYHEETRETL | | | | | |
| | | ILDSEITIENDP | | | | | |
| | | ESEKRQKLFPS | | | | | |
| | | KGE | | | | | |
| E9LVH7 | 15 | MANPYERGPN | Tha_Cut1 | PF12740; | IPR029058; | 3.1.1.101; | Polyesterase- |
| | | PTDALLEASSG | | | IPR041127; | 3.1.1.74 | lipase- |
| | | PFSVSEENVSR | | | | | cutinase |
| | | LSASGFGGGTI | | | | | |
| | | YYPRENNTYG | | | | | |
| | | AVAISPGYTGT | | | | | |
| | | EASIAWLGGRI | | | | | |
| | | ASHGFVVITID | | | | | |
| | | TITTLDQPDSR | | | | | |
| | | AEQLNAALNH | | | | | |
| | | MINRASSTVRS | | | | | |
| | | RIDSSRLAVM | | | | | |
| | | GHSMGGGGTP | | | | | |
| | | RLASQRPDLK | | | | | |
| | | AAIPLTPWHL | | | | | |
| | | NKNRSSVTVP | | | | | |
| | | TLIIGADLDTIA | | | | | |
| | | PVATHAKPFY | | | | | |
| | | NSLPSSISKAY | | | | | |
| | | LELDGATHFA | | | | | |
| | | PNIPNKIIGKYS | | | | | |
| | | VAWLKRFVD | | | | | |
| | | NDTRYTQFLC | | | | | |
| | | PGPRDGLFGE | | | | | |
| | | VEEYCSTCPF | | | | | |
| E9LVH8 | 16 | MANPYERGPN | The_Cut1 | PF12740; | IPR029058; | 3.1.1.101; | Polyesterase- |
| | | PTDALLEASSG | | | IPR041127; | 3.1.1.74 | lipase- |
| | | PFSVSEENVSR | | | | | cutinase |
| | | LSASGFGGGTI | | | | | |
| | | YYPRENNTYG | | | | | |
| | | AVAISPGYTGT | | | | | |
| | | EASIAWLGERI | | | | | |
| | | ASHGFVVITID | | | | | |
| | | TITTLDQPDSR | | | | | |
| | | AEQLNAALNH | | | | | |
| | | MINRASSTVRS | | | | | |
| | | RIDSSRLAVM | | | | | |
| | | GHSMGGGGTL | | | | | |
| | | RLASQRPDLK | | | | | |
| | | AAIPLTPWHL | | | | | |
| | | NKNWSSVTVP | | | | | |
| | | TLIIGADLDTIA | | | | | |
| | | PVATHAKPFY | | | | | |
| | | NSLPSSISKAY | | | | | |
| | | LELDGATHFA | | | | | |
| | | PNIPNKIIGKYS | | | | | |
| | | VAWLKRFVD | | | | | |
| | | NDTRYTQFLC | | | | | |
| | | PGPRDGLFGE | | | | | |
| | | VEEYRSTCPF | | | | | |
| E9LVH9 | 17 | MANPYERGPN | The_Cut2 | PF12740; | IPR029058; | 3.1.1.101; | Polyesterase- |
| | | PTDALLEARS | | | IPR041127; | 3.1.1.74 | lipase- |
| | | GPFSVSEERAS | | | | | cutinase |
| | | RFGADGFGGG | | | | | |
| | | TIYYPRENNTY | | | | | |
| | | GAVAISPGYT | | | | | |
| | | GTQASVAWLG | | | | | |
| | | ERIASHGFVVI | | | | | |
| | | TIDTNTTLDQP | | | | | |
| | | DSRARQLNAA | | | | | |
| | | LDYMINDASS | | | | | |
| | | AVRSRIDSSRL | | | | | |
| | | AVMGHSMGG | | | | | |
| | | GGTLRLASQR | | | | | |
| | | PDLKAAIPLTP | | | | | |
| | | WHLNKNWSS | | | | | |
| | | VRVPTLIIGAD | | | | | |
| | | LDTIAPVLTHA | | | | | |
| | | RPFYNSLPTSIS | | | | | |
| | | KAYLELDGAT | | | | | |
| | | HFAPNIPNKIIG | | | | | |
| | | KYSVAWLKRF | | | | | |
| | | VDNDTRYTQF | | | | | |
| | | LCPGPRDGLF | | | | | |
| | | GEVEEYRSTCP | | | | | |
| | | F | | | | | |
| F7IX06 | 18 | MSVTTPRRETS | Est119/ | PF12740; | IPR029058; | 3.1.1.101; | Polyesterase- |
| | | LLSRALRATA | Ta_Cut | | IPR041127; | 3.1.1.74 | lipase- |
| | | AAATAVVATV | | | | | cutinase |
| | | ALAAPAQAAN | | | | | |
| | | PYERGPNPTES | | | | | |
| | | MLEARSGPFS | | | | | |
| | | VSEERASRFG | | | | | |
| | | ADGFGGGTIY | | | | | |
| | | YPRENNTYGA | | | | | |
| | | IAISPGYTGTQ | | | | | |
| | | SSIAWLGERIA | | | | | |
| | | SHGFVVIAIDT | | | | | |
| | | NTTLDQPDSR | | | | | |
| | | ARQLNAALDY | | | | | |
| | | MLTDASSAVR | | | | | |
| | | NRIDASRLAV | | | | | |
| | | MGHSMGGGG | | | | | |
| | | TLRLASQRPDL | | | | | |
| | | KAAIPLTPWH | | | | | |
| | | LNKSWRDITV | | | | | |
| | | PTLIIGAEYDTI | | | | | |
| | | ASVTLHSKPFY | | | | | |
| | | NSIPSPTDKAY | | | | | |
| | | LELDGASHFA | | | | | |
| | | PNITNKTIGMY | | | | | |
| | | SVAWLKRFVD | | | | | |
| | | EDTRYTQFLCP | | | | | |
| | | GPRTGLLSDV | | | | | |
| | | EEYRSTCPF | | | | | |
| G2RAE6 | 19 | MKFLPILCAA | TtcutA | PF01083; | IPR029058; | 3.1.1.74 | Cutinase |
| | | GLAAAAPTQP | | | IPR000675; | | |
| | | AGEAAVEARQ | | | IPR043579; | | |
| | | LFSDTANDLE | | | IPR011150; | | |
| | | NGVSSNCPKVI | | | | | |
| | | FICARGSTETG | | | | | |
| | | NLGSSVCPEV | | | | | |
| | | ANGLKNYYPN | | | | | |
| | | QLWVQGVGG | | | | | |
| | | AYTADLASNA | | | | | |
| | | LPGGTSTAAM | | | | | |
| | | QEAANMFNLA | | | | | |
| | | QQKCPNASVA | | | | | |
| | | AGGYSQGTAV | | | | | |
| | | VAGGIQSLSA | | | | | |
| | | AAKDQIKGVV | | | | | |
| | | LFGYTQAQQN | | | | | |
| | | HDTIPNFPVDK | | | | | |
| | | TMIFCAQGDL | | | | | |
| | | VCNGTLIVTA | | | | | |
| | | AHFSYITNGD | | | | | |
| | | ASTKGPAWLH | | | | | |
| | | EKIGDA | | | | | |
| G9BY57 | 20 | MDGVLWRR | LCC | PF12695; | IPR029058; | 3.1.1.101; | Polyesterase- |
| | | TAALMAALLA | | | IPR029059; | 3.1.1.74 | lipase- |
| | | LAAWALVWA | | | | | cutinase |
| | | SPSVEAQSNPY | | | | | |
| | | QRGPNPTRSA | | | | | |
| | | LTADGPFSVA | | | | | |
| | | TYTVSRLSVSG | | | | | |
| | | FGGGVIYYPT | | | | | |
| | | GTSLTFGGIAM | | | | | |
| | | SPGYTADASSL | | | | | |
| | | AWLGRRLASH | | | | | |
| | | GFVVLVINTNS | | | | | |
| | | RFDYPDSRAS | | | | | |
| | | QLSAALNYLR | | | | | |
| | | TSSPSAVRARL | | | | | |
| | | DANRLAVAGH | | | | | |
| | | SMGGGGTLRI | | | | | |
| | | AEQNPSLKAA | | | | | |
| | | VPLTPWHTDK | | | | | |
| | | TFNTSVPVLIV | | | | | |
| | | GAEADTVAPV | | | | | |
| | | SQHAIPFYQNL | | | | | |
| | | PSTTPKVYVEL | | | | | |
| | | DNASHFAPNS | | | | | |
| | | NNAAISVYTIS | | | | | |
| | | WMKLWVDND | | | | | |
| | | TRYRQFLCNV | | | | | |
| | | NDPALSDFRT | | | | | |
| | | NNRHCQ | | | | | |
| H6WX58 | 21 | MANPYERGPN | Thh_Est | PF12146; | IPR029058; | | Polyesterase- |
| | | PTNSSIEALRG | | | IPR022742; | | lipase- |
| | | PFRVDEERVSR | | | | | cutinase |
| | | LQARGFGGGT | | | | | |
| | | IYYPTDNNTFG | | | | | |
| | | AVAISPGYTGT | | | | | |
| | | QSSISWLGERL | | | | | |
| | | ASHGFVVMTI | | | | | |
| | | DTNTTLDQPD | | | | | |
| | | SRASQLDAAL | | | | | |
| | | DYMVEDSSYS | | | | | |
| | | VRNRIDSSRLA | | | | | |
| | | AMGHSMGGG | | | | | |
| | | GTLRLAERRP | | | | | |
| | | DLQAAIPLTP | | | | | |
| | | WHTDKTWGS | | | | | |
| | | VRVPTLIIGAE | | | | | |
| | | NDTIASVRSHS | | | | | |
| | | EPFYNSLPGSL | | | | | |
| | | DKAYLELDGA | | | | | |
| | | SHFAPNLSNTT | | | | | |
| | | IAKYSISWLKR | | | | | |
| | | FVDDDTRYTQ | | | | | |
| | | FLCPGPSTGW | | | | | |
| | | GSDVEEYRST | | | | | |
| | | CPF | | | | | |
| P00590 | 22 | MKFFALTTLL | FsC | PF01083; | IPR029058; | 3.1.1.74 | Cutinase |
| | | AATASALPTS | | | IPR000675; | | |
| | | NPAQELEARQ | | | IPR043580; | | |
| | | LGRTTRDDLIN | | | IPR043579; | | |
| | | GNSASCRDVIF | | | IPR011150; | | |
| | | IYARGSTETGN | | | | | |
| | | LGTLGPSIASN | | | | | |
| | | LESAFGKDGV | | | | | |
| | | WIQGVGGAYR | | | | | |
| | | ATLGDNALPR | | | | | |
| | | GTSSAAIREML | | | | | |
| | | GLFQQANTKC | | | | | |
| | | PDATLIAGGYS | | | | | |
| | | QGAALAAASI | | | | | |
| | | EDLDSAIRDKI | | | | | |
| | | AGTVLFGYTK | | | | | |
| | | NLQNRGRIPN | | | | | |
| | | YPADRTKVFC | | | | | |
| | | NTGDLVCTGS | | | | | |
| | | LIVAAPHLAY | | | | | |
| | | GPDARGPAPE | | | | | |
| | | FLIEKVRAVRG | | | | | |
| | | SA | | | | | |
| P19833 | 23 | MFIMIKKSELA | MoPE | | IPR029058; | 3.1.1.3 | Polyesterase- |
| | | KAIIVTGALVF | | | | | lipase- |
| | | SIPTLAEVTLS | | | | | cutinase |
| | | ETTVSSIKSEA | | | | | |
| | | TVSSTKKALP | | | | | |
| | | ATPSDCIADSK | | | | | |
| | | ITAVALSDTRD | | | | | |
| | | NGPFSIRTKRIS | | | | | |
| | | RQSAKGFGGG | | | | | |
| | | TIHYPTNASGC | | | | | |
| | | GLLGAIAVVP | | | | | |
| | | GYVSYENSIK | | | | | |
| | | WWGPRLASW | | | | | |
| | | GFVVITINTNSI | | | | | |
| | | YDDPDSRAAQ | | | | | |
| | | LNAALDNMIA | | | | | |
| | | DDTVGSMIDP | | | | | |
| | | KRLGAIGWSM | | | | | |
| | | GGGGALKLAT | | | | | |
| | | ERSTVRAIMPL | | | | | |
| | | APYHDKSYGE | | | | | |
| | | VKTPTLVIACE | | | | | |
| | | DDRIAETKKY | | | | | |
| | | ANAFYKNAIG | | | | | |
| | | PKMKVEVNN | | | | | |
| | | GSHFCPSYRFN | | | | | |
| | | EILLSKPGIAW | | | | | |
| | | MQRYINNDTR | | | | | |
| | | FDKFLCANEN | | | | | |
| | | YSKSPRISAYD | | | | | |
| | | YKDCP | | | | | |
| Q2MDG4 | 24 | MKGIAALVTA | SterylEst | PF00135; | IPR029058; | 3.1.1.- | Funal_carbox |
| | | ALLGRAVAAP | | | IPR002018; | | ylesterase_lip |
| | | PEPPTNVVEKR | | | IPR019826; | | ase |
| | | AAPTVEISTGT | | | IPR019819; | | |
| | | IVGTTRLATEA | | | | | |
| | | FNGIPFALPPV | | | | | |
| | | GQLRLKPPVR | | | | | |
| | | LNSSLGVFDAS | | | | | |
| | | GIAPACPQFLA | | | | | |
| | | DSDSNEFLAQ | | | | | |
| | | VINTVTGLPFF | | | | | |
| | | QKALKISEDCL | | | | | |
| | | TINVIRPKGTK | | | | | |
| | | AGDKLPVLFW | | | | | |
| | | IYGGGFELGW | | | | | |
| | | SSMYDGGPLV | | | | | |
| | | TNAIGFGKPFI | | | | | |
| | | FVAVNYRVAG | | | | | |
| | | FGFMPGKEILA | | | | | |
| | | DGAANLGHLD | | | | | |
| | | QRMGLEWVA | | | | | |
| | | DNIAAFGGDP | | | | | |
| | | DKVTIWGESA | | | | | |
| | | GAISVLNQMA | | | | | |
| | | LFDGDHTYKG | | | | | |
| | | KPLFRGAIMNS | | | | | |
| | | GSIVPADPVDC | | | | | |
| | | PKGQEIYDQV | | | | | |
| | | VAKAGCAGAS | | | | | |
| | | DTLACLRELP | | | | | |
| | | YEKFLDAANS | | | | | |
| | | VPAILSYNSVA | | | | | |
| | | LSYLPRPDGK | | | | | |
| | | VLTKSPDVLIK | | | | | |
| | | EGKYAAVPMII | | | | | |
| | | GDQEDEGTLF | | | | | |
| | | SLFQPNITNSE | | | | | |
| | | KLVTYLKDLF | | | | | |
| | | FHGASREQLE | | | | | |
| | | GLVDRYPARIS | | | | | |
| | | AGSPFRTGLLN | | | | | |
| | | EIYPGFKRLAA | | | | | |
| | | ILGDLVFTLTR | | | | | |
| | | RVFLEDATRV | | | | | |
| | | NPTVPAWSYL | | | | | |
| | | ASYDYGTPILG | | | | | |
| | | TFHGSDLLQV | | | | | |
| | | FFGILPNYASR | | | | | |
| | | SIQSYYANFVY | | | | | |
| | | NLDPNDASGG | | | | | |
| | | TSGKSKVAEE | | | | | |
| | | WPRWTADDR | | | | | |
| | | TLIQFFANRNG | | | | | |
| | | YLKDDFRSEA | | | | | |
| | | AEYIGAHVDY | | | | | |
| | | LHI | | | | | |
| Q6A0I3 | 25 | MAVMTPRRER | BTA-2 | PF12740; | IPR029058; | 3.1.1.101; | Polyesterase- |
| | | SSLLSRALRFT | | | IPR041127; | 3.1.1.74 | lipase- |
| | | AAAATALVTA | | | | | cutinase |
| | | VSLAAPAHAA | | | | | |
| | | NPYERGPNPT | | | | | |
| | | DALLEARSGPF | | | | | |
| | | SVSEERASRFG | | | | | |
| | | ADGFGGGTIY | | | | | |
| | | YPRENNTYGA | | | | | |
| | | VAISPGYTGTQ | | | | | |
| | | ASVAWLGKRI | | | | | |
| | | ASHGFVVITID | | | | | |
| | | TNTTLDQPDS | | | | | |
| | | RARQLNAALD | | | | | |
| | | YMINDASSAV | | | | | |
| | | RSRIDSSRLAV | | | | | |
| | | MGHSMGGGG | | | | | |
| | | SLRLASQRPDL | | | | | |
| | | KAAIPLTPWH | | | | | |
| | | LNKNWSSVRV | | | | | |
| | | PTLIIGADLDTI | | | | | |
| | | APVLTHARPF | | | | | |
| | | YNSLPTSISKA | | | | | |
| | | YLELDGATHF | | | | | |
| | | APNIPNKIIGK | | | | | |
| | | YSVAWLKRFV | | | | | |
| | | DNDTRYTQFL | | | | | |
| | | CPGPRDGLFG | | | | | |
| | | EVEEYRSTCPF | | | | | |
| R4YKL9 | 26 | MNKSILKKLSF | PET5 | PF01738; | IPR029058; | | Polyesterase- |
| | | GTSVLLVSMN | | | IPR002925; | | lipase- |
| | | ALSWTPSPTPN | | | | | cutinase |
| | | PDPIPDPTPCQ | | | | | |
| | | DDCDFTRGPN | | | | | |
| | | PTPSSLEASTG | | | | | |
| | | PYSVATRSVA | | | | | |
| | | SSVSGFGGGTL | | | | | |
| | | HYPTNTTGTM | | | | | |
| | | GALAVVPGFLL | | | | | |
| | | QESSIDFWGPK | | | | | |
| | | LASHGFVVITI | | | | | |
| | | SANSGFDQPA | | | | | |
| | | SRATQLGRAL | | | | | |
| | | DYVINQSNGS | | | | | |
| | | NSPISGMVDTT | | | | | |
| | | RLGVVGWSM | | | | | |
| | | GGGGALQLAS | | | | | |
| | | GDRLSAAIPIA | | | | | |
| | | PWNQGGNRFD | | | | | |
| | | QIETPTLVIAC | | | | | |
| | | ENDVVASVNS | | | | | |
| | | HASPFYNRIPS | | | | | |
| | | TTDKAYLEIN | | | | | |
| | | GGSHFCANDG | | | | | |
| | | GSIGGLLGKY | | | | | |
| | | GVSWMKRFID | | | | | |
| | | NDLRYDAFLC | | | | | |
| | | GPDHAANRSV | | | | | |
| | | SEYRDTCNY | | | | | |
| W0TJ64 | 27 | MRIRRQAGTG | Cut190 | PF03403; | IPR029058; | | Polyesterase- |
| | | ARASMARAIG | | | | | lipase- |
| | | VMTTALAVLV | | | | | cutinase |
| | | GAVGGVAGA | | | | | |
| | | EVSTAQDNPY | | | | | |
| | | ERGPDPTEDSI | | | | | |
| | | EAIRGPFSVAT | | | | | |
| | | ERVSSFASGFG | | | | | |
| | | GGTIYYPRETD | | | | | |
| | | EGTFGAVAVA | | | | | |
| | | PGFTASQGSM | | | | | |
| | | SWYGERVASQ | | | | | |
| | | GFIVFTIDTNT | | | | | |
| | | RLDQPGQRGR | | | | | |
| | | QLLAALDYLV | | | | | |
| | | ERSDRKVRER | | | | | |
| | | LDPNRLAVMG | | | | | |
| | | HSMGGGGSLE | | | | | |
| | | ATVMRPSLKA | | | | | |
| | | SIPLTPWNLDK | | | | | |
| | | TWGQVQVPTF | | | | | |
| | | IIGAELDTIASV | | | | | |
| | | RTHAKPFYESL | | | | | |
| | | PSSLPKAYME | | | | | |
| | | LDGATHFAPNI | | | | | |
| | | PNTTIAKYVIS | | | | | |
| | | WLKRFVDEDT | | | | | |
| | | RYSQFLCPNPT | | | | | |
| | | DRAIEEYRSTC | | | | | |
| | | PY | | | | | |
| X0BTD8 | 28 | MKFSIISTLLA | FoCut5a | PF01083; | IPR029058; | 3.1.1.74 | Cutinase |
| | | ATASALPAGQ | | | IPR000675; | | |
| | | DAAALEARQL | | | IPR043580; | | |
| | | GGSITRNDLA | | | IPR043579; | | |
| | | NGNSGSCPGVI | | | IPR011150; | | |
| | | FIYARGSTESG | | | | | |
| | | NLGTLGPRVA | | | | | |
| | | SKLEAKYGKN | | | | | |
| | | GVWIQGVGGA | | | | | |
| | | YRATLGDNAL | | | | | |
| | | PRGTSSAAIRE | | | | | |
| | | MLGHFSDANQ | | | | | |
| | | KCPDAVLIAG | | | | | |
| | | GYSQGAALAA | | | | | |
| | | ASVTDVDAGI | | | | | |
| | | REKIAGVVLF | | | | | |
| | | GYTKNLQNRG | | | | | |
| | | KIPSYPEDRTK | | | | | |
| | | VFCNTGDLVC | | | | | |
| | | TGSLIVAAPHL | | | | | |
| | | AYQSAASGAA | | | | | |
| | | PEFLIQKADAA | | | | | |
| | | GAA | | | | | |
| A0A031MKR8 | 29 | MINKNLSQSLL | PauzCut | PF01738 | IPR029058; | | Polyesterase- |
| | | AMMAAGALL | | | IPR002925 | | lipase- |
| | | LSSSAFAVNPP | | | | | cutinase |
| | | TDGPTDPDQA | | | | | |
| | | YERGPDPSVA | | | | | |
| | | FLEAPTGPHSV | | | | | |
| | | RTSRVSGLVS | | | | | |
| | | GFGGGTIHYPT | | | | | |
| | | GTTGTMAAIV | | | | | |
| | | VIPGFVSAESSI | | | | | |
| | | EWWGPKLAS | | | | | |
| | | HGFVVMTIDT | | | | | |
| | | NTGFDQPPSR | | | | | |
| | | ARQINNALDY | | | | | |
| | | LVSQNTSRTSP | | | | | |
| | | VNGMIDTERL | | | | | |
| | | GVIGWSMGGG | | | | | |
| | | GTLRVASEGRI | | | | | |
| | | KAAIPLAPWD | | | | | |
| | | TTRFRGVQAP | | | | | |
| | | TLIFACESDLIA | | | | | |
| | | PVRSHASPFYN | | | | | |
| | | QLPDDIDKAY | | | | | |
| | | VEINNGSHYC | | | | | |
| | | ANGGGLNND | | | | | |
| | | VLSRFGVSWM | | | | | |
| | | KRFLDNDTRY | | | | | |
| | | SQFLCGPNHES | | | | | |
| | | DRNISEYRGN | | | | | |
| | | CPY | | | | | |
| A0A078MGG8 | 30 | MINKKLSQTL | PsCut | PF01738 | IPR029058; | | Polyesterase- |
| | | LSMAAAGAL | | | IPR002925 | | lipase- |
| | | MFSASVFATN | | | | | cutinase |
| | | PPTDEPTNPGQ | | | | | |
| | | SYERGPDPTV | | | | | |
| | | AFLEASSGPYS | | | | | |
| | | VRTSRVSGLV | | | | | |
| | | SGFGGGTIHYP | | | | | |
| | | TGTTGTMAAI | | | | | |
| | | VVIPGFVSAES | | | | | |
| | | SIDWWGPKLA | | | | | |
| | | SHGFVVMTID | | | | | |
| | | TNTGFDQPPSR | | | | | |
| | | ARQINNALDY | | | | | |
| | | LVDQNSSRTSP | | | | | |
| | | VNGMIDTERL | | | | | |
| | | GVIGWSMGGG | | | | | |
| | | GTLRVASEGRI | | | | | |
| | | KAAIPLAPWD | | | | | |
| | | TTRFSGVQAPT | | | | | |
| | | LIFACESDLIAP | | | | | |
| | | VRSHASPFYN | | | | | |
| | | QLPNDIDKAY | | | | | |
| | | VEINNGSHYC | | | | | |
| | | ANGGGLNND | | | | | |
| | | VLSRFGVSWM | | | | | |
| | | KRFLDNDTRY | | | | | |
| | | SQFLCGPRHES | | | | | |
| | | DRKISEYRGN | | | | | |
| | | CPY | | | | | |
| A0A1Z2SIQ1 | 31 | MMNVLTKCK | PET6, | | IPR029058; | | Polyesterase- |
| | | LALGILAIFFSL | BSQ33_03270 | | | | lipase- |
| | | PSFAVPCSDCS | | | | | cutinase |
| | | NGFERGQVPR | | | | | |
| | | VDQLESSRGP | | | | | |
| | | YSVKTINVSRL | | | | | |
| | | ARGFGGGTIH | | | | | |
| | | YSTESGGQQGI | | | | | |
| | | LAVVPGYVSLE | | | | | |
| | | GSIKWWGPRL | | | | | |
| | | ASWGFTVITID | | | | | |
| | | TNTIYDQPDSR | | | | | |
| | | ASQLSAAIDY | | | | | |
| | | VIDKGNDRSSP | | | | | |
| | | IYGLVDPNRV | | | | | |
| | | GVIGWSMGGG | | | | | |
| | | GSLKLATDRKI | | | | | |
| | | DAVIPQAPWY | | | | | |
| | | LGLSRFSSITSP | | | | | |
| | | TMILACQADV | | | | | |
| | | VAPVSVHASR | | | | | |
| | | FYNQIPGTTPK | | | | | |
| | | AYFEIALGSHF | | | | | |
| | | CANTGYPSEDI | | | | | |
| | | LGRNGVAWM | | | | | |
| | | KRFIDKDERYT | | | | | |
| | | QFLCGQNFDS | | | | | |
| | | SLRVSEYRDN | | | | | |
| | | CSYY | | | | | |
| E8U721 | 32 | MSNGYSACPA | DSM 21211, | PF01738 | IPR029058; | 3.1.1.3 | Polyesterase- |
| | | PYDHRGMRK | PET1/ | | IPR002925 | | lipase- |
| | | HASLLAALPV | DmPETase | | | | cutinase |
| | | LLAACGTTTST | | | | | |
| | | LTTPAVTPQTL | | | | | |
| | | SGAATNPYER | | | | | |
| | | GPAPTTALLEA | | | | | |
| | | ARGPYATAST | | | | | |
| | | TVPRSSVSDFG | | | | | |
| | | GATIYYPTSTA | | | | | |
| | | DGTFGGVAISP | | | | | |
| | | GYTGTQASVA | | | | | |
| | | WLGPRLASHG | | | | | |
| | | FVVIVIDTLSR | | | | | |
| | | YDYPSSRGDQ | | | | | |
| | | LRAALRYLTT | | | | | |
| | | SSAVRTRVDA | | | | | |
| | | TRLAVMGHS | | | | | |
| | | MGGGGALEA | | | | | |
| | | AKDNPALKAA | | | | | |
| | | IPLTPWNTDKS | | | | | |
| | | WPELTTPTLIF | | | | | |
| | | GAQNDSVAPV | | | | | |
| | | SSHAIPFYTSL | | | | | |
| | | ASTLPKAYLEL | | | | | |
| | | RGASHGAPTS | | | | | |
| | | TNTTIAKYAV | | | | | |
| | | AWLKRFEDAD | | | | | |
| | | RRYDPFLCPTP | | | | | |
| | | AVSTLLSDARS | | | | | |
| | | TCPFN | | | | | |
| P41365 | 33 | MKLLSLTGVA | CalB; | | IPR029058 | 3.1.1.3 | Canar_LipB |
| | | GVLATCVAAT | lipase B | | | | |
| | | PLVKRLPSGSD | | | | | |
| | | PAFSQPKSVLD | | | | | |
| | | AGLTCQGASP | | | | | |
| | | SSVSKPILLVP | | | | | |
| | | GTGTTGPQSF | | | | | |
| | | DSNWIPLSTQL | | | | | |
| | | GYTPCWISPPP | | | | | |
| | | FMLNDTQVNT | | | | | |
| | | EYMVNAITAL | | | | | |
| | | YAGSGNNKLP | | | | | |
| | | VLTWSQGGLV | | | | | |
| | | AQWGLTFFPSI | | | | | |
| | | RSKVDRLMAF | | | | | |
| | | APDYKGTVLA | | | | | |
| | | GPLDALAVSA | | | | | |
| | | PSVWQQTTGS | | | | | |
| | | ALTTALRNAG | | | | | |
| | | GLTQIVPTTNL | | | | | |
| | | YSATDEIVQPQ | | | | | |
| | | VSNSPLDSSYL | | | | | |
| | | FNGKNVQAQA | | | | | |
| | | VCGPLFVIDHA | | | | | |
| | | GSLTSQFSYVV | | | | | |
| | | GRSALRSTTG | | | | | |
| | | QARSADYGIT | | | | | |
| | | DCNPLPANDL | | | | | |
| | | TPEQKVAAAA | | | | | |
| | | LLAPAAAAIV | | | | | |
| | | AGPKQNCEPD | | | | | |
| | | LMPYARPFAV | | | | | |
| | | GKRTCSGIVTP | | | | | |
| Q47RJ7 | 34 | MPPHAARPGP | Tfu_0882 | PF12740 | IPR029058; | 3.1.1.101; | Polyesterase- |
| | | AQNRRGRAM | | | IPR041127 | 3.1.1.74 | lipase- |
| | | AVITPRRERSS | | | | | cutinase |
| | | LLSRALRFTAA | | | | | |
| | | AATALVTAVS | | | | | |
| | | LAAPAHAANP | | | | | |
| | | YERGPNPTDA | | | | | |
| | | LLEARSGPFSV | | | | | |
| | | SEERASRFGA | | | | | |
| | | DGFGGGTIYY | | | | | |
| | | PRENNTYGAV | | | | | |
| | | AISPGYTGTQA | | | | | |
| | | SVAWLGERIA | | | | | |
| | | SHGFVVITIDT | | | | | |
| | | NTTLDQPDSR | | | | | |
| | | ARQLNAALDY | | | | | |
| | | MINDASSAVR | | | | | |
| | | SRIDSSRLAVM | | | | | |
| | | GHSMGGGGTL | | | | | |
| | | RLASQRPDLK | | | | | |
| | | AAIPLTPWHL | | | | | |
| | | NKNWSSVRVP | | | | | |
| | | TLIIGADLDTIA | | | | | |
| | | PVLTHARPFY | | | | | |
| | | NSLPTSISKAY | | | | | |
| | | LELDGATHFA | | | | | |
| | | PNIPNKIIGKYS | | | | | |
| | | VAWLKRFVD | | | | | |
| | | NDTRYTQFLC | | | | | |
| | | PGPRDGLFGE | | | | | |
| | | VEEYRSTCPF | | | | | |
| Q8RR62 | 35 | MHLPRSRWDI | AdCut | PF01738 | IPR029058; | | Polyesterase- |
| | | PFKEETTMTH | | | IPR002925 | | lipase- |
| | | HFSVRALLAA | | | | | cutinase |
| | | GALLASAAVS | | | | | |
| | | AQTNPYERGP | | | | | |
| | | APTTSSLEASR | | | | | |
| | | GPFSYQSFTVS | | | | | |
| | | RPSGYRAGTV | | | | | |
| | | YYPTNAGGPV | | | | | |
| | | GAIAIVPGFTA | | | | | |
| | | RQSSINWWGP | | | | | |
| | | RLASHGFVVIT | | | | | |
| | | IDTNSTLDQPD | | | | | |
| | | SRSRQQMAAL | | | | | |
| | | SQVATLSRTSS | | | | | |
| | | SPIYNKVDTSR | | | | | |
| | | LGVMGWSMG | | | | | |
| | | GGGSLISARNN | | | | | |
| | | PSIKAAAPQAP | | | | | |
| | | WSASKNFSSL | | | | | |
| | | TVPTLIIACEN | | | | | |
| | | DTIAPVNQHA | | | | | |
| | | DTFYDSMSRN | | | | | |
| | | PREFLEINNGS | | | | | |
| | | HSCANSGNSN | | | | | |
| | | QALLGKKGVA | | | | | |
| | | WMKRFMDND | | | | | |
| | | RRYTSFACSNP | | | | | |
| | | NSYNVSDFRV | | | | | |
| | | AACN | | | | | |
| W6R2Y2 | 36 | MRAWWLSGA | PpEst (tesA) | PF13472 | IPR013830; | 3.1.2.- | |
| | | LALMFWAQG | PHL3 | | IPR036514 | | |
| | | AVAGTLLVVG | | | | | |
| | | DSISAAFGLDS | | | | | |
| | | RQGWVALLEK | | | | | |
| | | RLSEEGFEHSV | | | | | |
| | | VNASISGDTSA | | | | | |
| | | GGAARLSALL | | | | | |
| | | AEHKPELVIIE | | | | | |
| | | LGGNDGLRGQ | | | | | |
| | | PPAQLQQNLA | | | | | |
| | | SMVEQSQQAG | | | | | |
| | | AKVLLLGMKL | | | | | |
| | | PPNYGVRYTT | | | | | |
| | | AFAQVFTDLA | | | | | |
| | | EQKQVSLVPFF | | | | | |
| | | LEGVGGVPGM | | | | | |
| | | MQADGIHPAE | | | | | |
| | | AAQEILLDNV | | | | | |
| | | WPTLKPML | | | | | |
| LT571442.1 | 37 | MENPYERGPD | | | | | |
| | | PTESSIEAVRG | | | | | |
| | | PFAVAQTTVS | | | | | |
| | | RLQADGFGGG | | | | | |
| | | TIYYPTDTSQG | | | | | |
| | | TFGAVAISPGF | | | | | |
| | | TAGQESIAWL | | | | | |
| | | GPRIASQGFVV | | | | | |
| | | ITIDTITRLDQP | | | | | |
| | | DSRGRQLQAA | | | | | |
| | | LDHLRTNSVV | | | | | |
| | | RNRIDPNRMA | | | | | |
| | | VMGHSMGGG | | | | | |
| | | GALSAAANNT | | | | | |
| | | SLEAAIPLQG | | | | | |
| | | WHTRKNWSS | | | | | |
| | | VRTPTLVVGA | | | | | |
| | | QLDTIAPVSSH | | | | | |
| | | SEAFYNSLPSD | | | | | |
| | | LDKAYMELRG | | | | | |
| | | ASHFVSNTPDT | | | | | |
| | | TTAKYSIAWL | | | | | |
| | | KRFVDNDLRY | | | | | |
| | | EQFLCPAPDDF | | | | | |
| | | AISEYRATCPF | | | | | |
| LT571446.1 | 38 | MANPYERGPD | PHL7 | | | | |
| | | PTESSIEAVRG | | | | | |
| | | PFAVAQTTVS | | | | | |
| | | RLQADGFGGG | | | | | |
| | | TIYYPTDTSQG | | | | | |
| | | TFGAVAISPGF | | | | | |
| | | TAGQESIAWL | | | | | |
| | | GPRIASQGFVV | | | | | |
| | | ITIDTITRLDQP | | | | | |
| | | DSRGRQLQAA | | | | | |
| | | LDHLRTNSVV | | | | | |
| | | RNRIDPNRMA | | | | | |
| | | VMGHSMGGG | | | | | |
| | | GALSAAANNT | | | | | |
| | | SLEAAIPLQG | | | | | |
| | | WHTRKNWSS | | | | | |
| | | VRTPTLVVGA | | | | | |
| | | QLDTIAPVSSH | | | | | |
| | | SEAFYNSLPSD | | | | | |
| | | LDKAYMELRG | | | | | |
| | | ASHLVSNTPD | | | | | |
| | | TTTAKYSIAW | | | | | |
| | | LKRFVDDDLR | | | | | |
| | | YEQFLCPAPD | | | | | |
| | | DFAISEYRSTC | | | | | |
| | | PF | | | | | |

In some embodiments, the polymer-degrading enzyme is a HiC. In some embodiments, the amino acid sequence of the HiC enzyme is set forth as: SEQ ID NO: 1 or a fragment thereof. In some embodiments, the polymer-degrading enzyme is a variant of HiC having an insertion, deletion, or amino acid substitution at any one or more of the following positions: 1, 2, 5, 43, 55, 79, 115, 161, 181, 182, G8, S116, S119, A4, T29, L167, S48, N15, A88, N91, A130, T166, Q139, I169, I178 or R189 compared with the amino acid sequence of the HiC enzyme is set forth as: SEQ ID NO: 1. In some embodiments, the polymer-degrading enzyme is a variant of HiC having an amino acid substitution at up to 1, 2, 3, 4, 5, 6, 7, 8, 9, 10, 11, 12, 13, 14, 15, 16, 17, 18, 19, or 20 sites selected from the previous list. In some embodiments, the polymer-degrading enzyme is a variant of HiC, in which the variant of HiC has at least 85%, 86%, 87%, 88%, 89%, 90%, 91%, 92%, 93%, 94%, 95%, 96%, 97%, 98%, or 99% identity compared with the amino acid sequence of the HiC enzyme is set forth as: SEQ ID NO: 1.

In some embodiments, the polymer-degrading enzyme is a leaf-branch compost cutinase (LCC). In some embodiments, the amino acid sequence of the LCC enzyme is set forth as: SEQ ID NO: 20 or a fragment thereof. In some embodiments, the polymer-degrading enzyme is a variant of LCC having an insertion, deletion, or amino acid substitution at any one or more of the following positions: D238, S283, E208, L237, N239, A207, A244, V63, S64, R65, L66, S67, V68, S69, G70, F71, G72, G73, G74, A138, L117, G88, L139, L142, L154, A156, L159, 189, M91, L105, L109, A162, V185, L187, L203, V205, P231, V233, V235, V254, Y255, T256, S258, W259, M260, L274, T287, N288, H291, S36, Y39, Q40, R41, N44, S48, T51, S57, T60, Y61, Y78, S83, T85, R107, S133, N140, R143, S148, N157, S180, K182, T195, N197, S216, Q224, N225, S228, T229, S247, N248, N266, T268, R271, Q272, N276, N278, N289, R290, Q293, V2121, Y127G, Y127P, F2431, F243W, T96M, V205I, D238C, S283C, E208R, E208A, N239D, or L237R compared with the amino acid sequence of the LCC enzyme is set forth as: SEQ ID NO: 20. For example, in some embodiments, the polymer-degrading enzyme is a variant of LCC having one or more of the following substitutions F2431, D238C, S283C, and Y127G compared with the amino acid sequence of the LCC enzyme is set forth as: SEQ ID NO: 20. In some embodiments, the polymer-degrading enzyme comprises or consists of an amino acid sequence corresponding to positions 36 to 258 of SEQ ID NO: 20. In some embodiments, the polymer-degrading enzyme comprises or consists of an amino acid sequence corresponding to positions 36 to 258 of SEQ ID NO: 20 with an insertion, deletion, or amino acid substitutions at any one or more of the corresponding positions of the previous lists. In some embodiments, the polymer-degrading enzyme is a variant of LCC having an amino acid substitution at up to 1, 2, 3, 4, 5, 6, 7, 8, 9, 10, 11, 12, 13, 14, 15, 16, 17, 18, 19, or 20 sites selected from the previous list. In some embodiments, the polymer-degrading enzyme is a variant of LCC, in which the variant of LCC has at least 85%, 86%, 87%, 88%, 89%, 90%, 91%, 92%, 93%, 94%, 95%, 96%, 97%, 98%, or 99% identity compared with the amino acid sequence of the LCC enzyme is set forth as: SEQ ID NO: 20.

In some embodiments, polymer-degrading enzymes can be engineered according to information in the following literary publications which are herein incorporated by reference in their entirety for all purposes: Dombkowski A, Sultana K Z, Craig D. Protein disulfide engineering. FEBS Letters Volume 588, Issue 2, 206-212. 2014; Liu Q, Xun G, Feng Y. The state-of-the-art strategies of protein engineering for enzyme stabilization. Biotechnol Adv. 2019 July-Aug;37 (4): 530-537. doi: 10.1016/j.biotechadv.2018.10.011. Epub 2018 Oct. 26. PMID: 31138425; Federica Rigoldi, Stefano Donini, Alberto Redaelli, Emilio Parisini, Alfonso Gautieri; Review: Engineering of thermostable enzymes for industrial applications. APL Bioeng. 1 Mar. 2018; 2 (1): 011501. doi.org/10.1063/1.4997367; Chen, K., Arnold, F. H. Engineering new catalytic activities in enzymes. Nat Catal 3, 203-213 (2020). doi.org/10.1038/s41929-019-0385-5; Robert Chapman and Martina H. Stenzel. All Wrapped up: Stabilization of Enzymes within Single Enzyme Nanoparticles. Journal of the American Chemical Society 2019 141 (7), 2754-2769. DOI: 10.1021/jacs.8b10338; Spence M, Kaczmarski J, Saunders J, Jackson C. Ancestral sequence reconstruction for protein engineers. Current Opinion in Structural Biology, Volume 69. 2021; Raquel A. Rocha, Robert E. Speight, and Colin Scott. Engineering Enzyme Properties for Improved Biocatalytic Processes in Batch and Continuous Flow.Organic Process Research & Development 2022 26 (7), 1914-1924. DOI: 10.1021/acs.oprd. 1c00424; Chowdhury, R, Maranas, C D. From directed evolution to computational enzyme engineering-A review. AIChE J. 2020; 66: e16847. doi.org/10.1002/aic.16847; and Ferreira P, Fernandes P A, Ramos M J. Modern computational methods for rational enzyme engineering. Chem Catalysis, Volume 2, Issue 10, 2481-2498. 2022.

In some embodiments, a polymer-degrading enzyme comprises one or more conservative amino acid substitutions relative to a reference sequence. Such conservative substitutions of amino acids include substitutions made amongst amino acids within the following groups: (a) M, I, L, V; (b) F, Y, W; (c) K, R, H; (d) A, G; (c) S, T; (f) Q, N; and (g) E, D. In general, a conservative amino acid substitution refers to an amino acid substitution that does not alter the relative charge or size characteristics of the protein in which the amino acid substitution is made. In some embodiments, the polymer-degrading enzyme comprises at least 1, 2, 3, 4, 5 or more amino acid substitutions within the active site of the enzyme. In some embodiments, the polymer-degrading enzyme comprises at least 1, 2, 3, 4, 5 or more amino acid substitutions outside the active site of the enzyme. In some embodiments, the polymer-degrading enzyme is a variant of an enzyme that comprises a substitution of one or more amino acids in or proximal to a divalent metal binding site of the enzyme with cystine amino acids to promote formation of a disulfide bridge, e.g., thereby increasing thermostability relative to the parent enzyme.

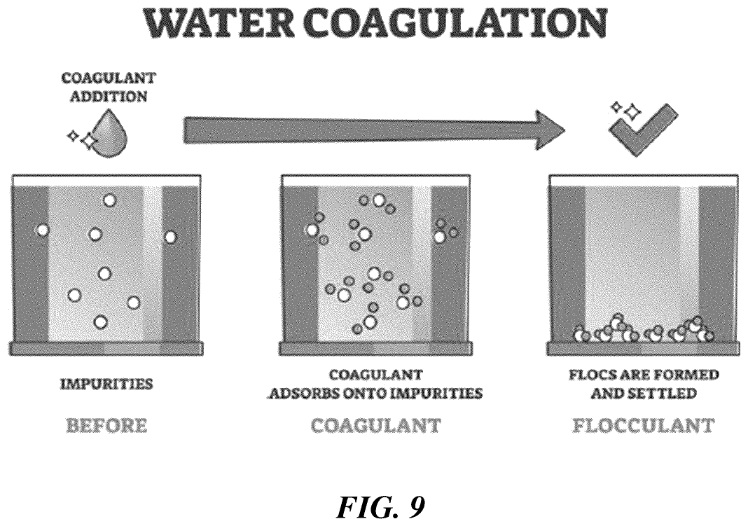

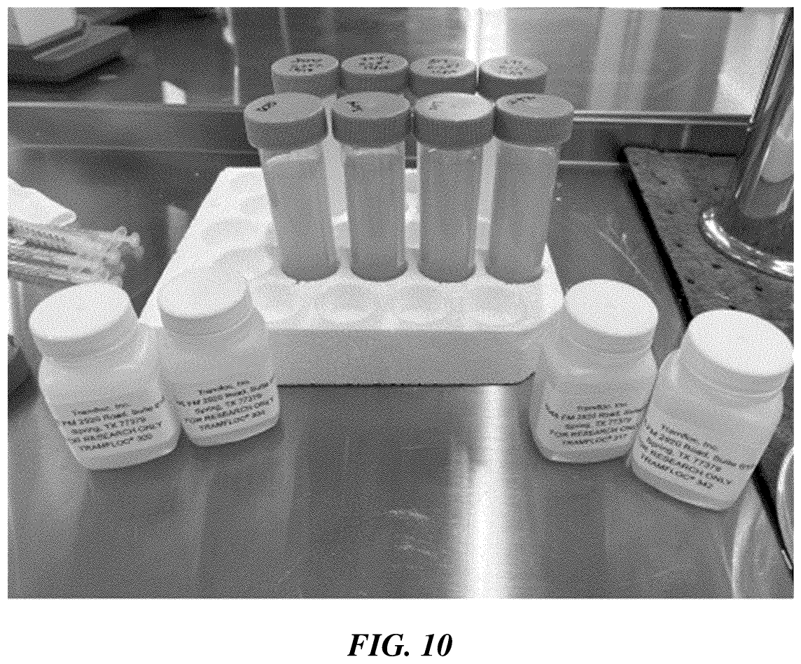

While removal of plastic from a fluid via a denatured polypeptide is described herein as a primary route to plastics agglomeration/removal, it is to be understood that auxiliary agents can be used in combination with the above. For example, nothing precludes use of an auxiliary agglomeration and/or flocculation agent (FIGS. 9-10). Those of ordinary skill in the art are aware of such agents and they can be used in combination with any aspect of this disclosure.

The following examples are intended to illustrate certain embodiments of the present invention, but do not exemplify the full scope of the invention.

### Example 1

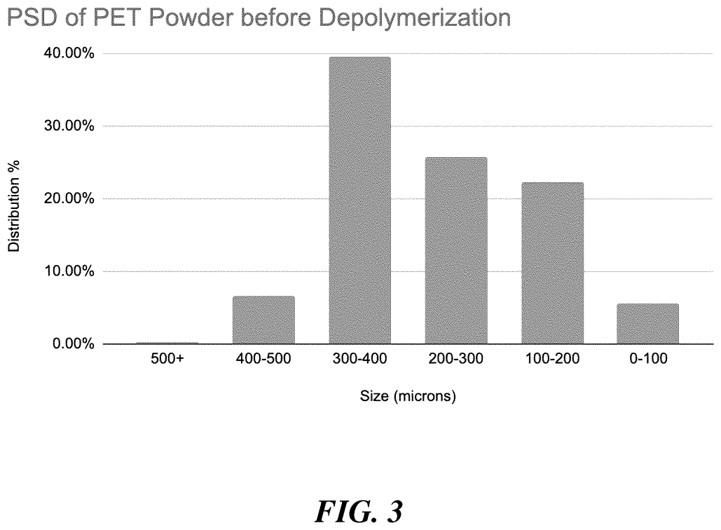

In this example, solid PET powder was enzymatically depolymerized in a bioreactor using Novozyme HIC 51032 (LOT L01332211, purchased from Strem Chemicals). The PET powder particle size distribution was measured by sieving using Cole Palmer 3″-diameter sieves with Retsch AS 200 Control Sieve Shaker. The particle size distribution is described in FIG. 3. The majority of the particles were in the range of 100 to 400 microns in diameter.

The reaction was carried out at 65° C. and with 10% solid loading of PET (30 g in 270 mL), 100 millimolar potassium phosphate buffer at pH 8.0, and 15 mL of enzyme solution as received. The pH of the reaction was controlled using a Raspberry Pi controlled system that doses 6 molar sodium hydroxide to maintain a pH of 8.0.

The reaction progression and kinetics can be understood by the dosing rate of the sodium hydroxide, as shown in FIG. 6B. The equation used to correlate sodium hydroxide dosed to reaction completion is shown below in Eq. 1:

$$\% Completion = \frac{\begin{matrix}Volume Base Dosed ( L ) * \\ Molarity ( \frac{Moles}{L} ) * 166.13 ( \frac{g}{mol} )\end{matrix}}{2 * Theoretical Yield} \qquad ( Eq . 1 )$$

Accordingly, since this example used 30 grams of PET, the theoretical yield of Terephthalic Acid was 25.92 grams. During the reaction, 46.18 mL of 6M sodium hydroxide was dosed over 69.4 hours which corresponds to a reaction completeness of 88.8%. The remaining product was an opaque liquid with residual PET particles in suspension.

### Example 2

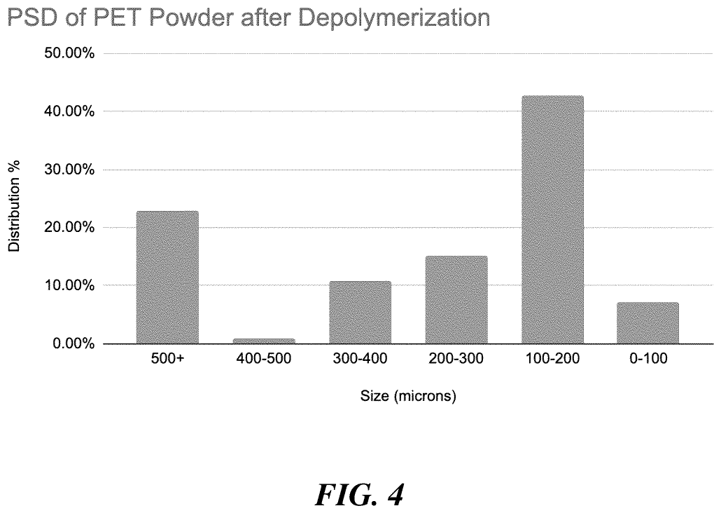

The reactor product from Example 1 comprised water, sodium terephthalate, ethylene glycol, unreacted PET particles, and other insoluble impurities. The 100 mL reactor product was filtered through a 8 micrometer filter (Whatman 40, Cytiva) in a buchner funnel using a vacuum pump (IKA MVP10 Basic). The filtration time for this product was timed using a stopwatch and measured to be 1 hour and 36 minutes. Collected solids were dried and weighed to yield 0.717 grams of unreacted solids. The solids were sieved using Cole Palmer 3″-diameter sieves with Retsch AS 200 Control Sieve Shaker to determine the particle size distribution, which is shown in FIG. 4. During sieving, material aggregates in clumps that do not pass through the 500 micrometer sieve easily. The shift in particle size towards smaller diameters is suspected to cause the long filtration times.

### Example 3

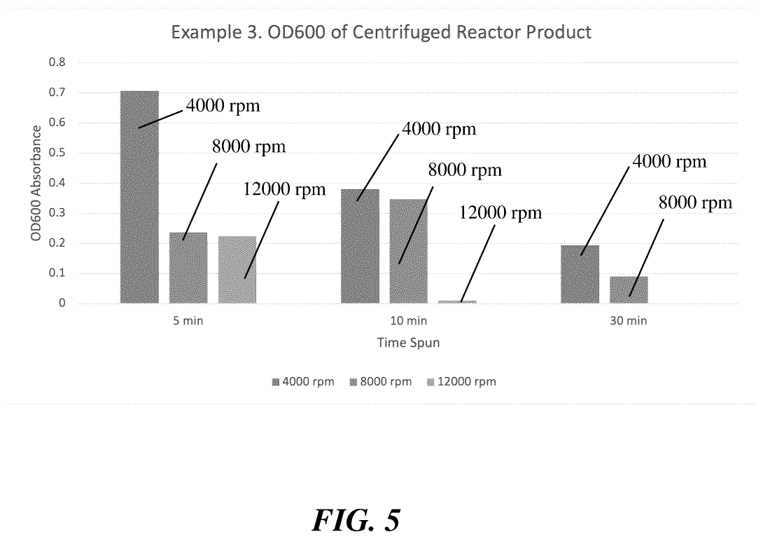

Reactor product generated from a reaction similar to Example 1 was split into 50 mL centrifuge tubes. It was found experimentally that using a centrifuge (Eppendorf Centrifuge 5920R) at an rpm of 12000 rpm or a force of 19802 relative centrifugal force for 10 minutes produced a clarified product, as measured (in triplicate) by OD600 of 1 mL of supernatant in a cuvette (Nanodrop OneC, ThermoFischer). However, such high rpm and spinning durations are not feasible using commercially available centrifuges at large scale. Test conditions and results are shown in FIG. 5.

### Example 4

To determine the enzyme concentration in the filtrate, filtered product from Example 2 was measured using a Bicinchoninic Acid Assay with Bovine Serum Albumin as a standard. The filtered product from Example 2 had an enzyme concentration of 1.92×10⁻⁵±0.12×10⁻⁵ M. The removal of enzymes present in the reactor product from the supernatant is important to avoid contamination of the terephthalic acid product and should be done prior to further processing.

### Example 5

100 mL of Filtered product from Example 2 was heated to above the melting temperature of Novozym HiC for 10 minutes with stirring. The stirring was then stopped, and the sample was allowed to rest for 30 minutes. A precipitate was formed and filtered on a 8 micrometer filter (Whatman 40, Cytiva) in a buchner funnel using a vacuum pump (IKA MVP10 Basic). The filter was dried and weighed. The retained solids weighed 181 milligrams. The filtrate was measured using the enzyme quantification method from Example 4. The enzyme concentration was determined to be 1.32×10⁻⁵±0.08×10⁻⁵ M.

### Comparative Example 1

Reactor product was generated in a similar manner as Example 1. The reactor product was heated to above the melting temperature of Novozym HiC (Tₘ=86 degrees Celsius as shown in FIG. 15) for 10 minutes with stirring. After stirring was stopped, the samples were allowed to rest for 30 minutes. Agglomerates of PET settled to the bottom of the vessel resulting in a clear supernatant. The reactor product was filtered on a 8 micrometer filter (Whatman 40, Cytiva) in a buchner funnel using a vacuum pump (IKA MVP10 Basic). The filtration time for this product was timed using a stopwatch and measured to be 19 seconds, which is lower than that in Example 2. The application of heat to the enzyme and PET mixture facilitated the filtering process. Filtered product was tested similar to Example 4 for enzyme concentration. The enzyme concentration of the product was 1.12×10⁵±0.04×10⁻⁵ M. Accordingly, in addition to reduced the duration of filtration, the enzyme concentration in the filtrate was lower than that of Example 4.

### Comparative Example 2

Reactor product was generated in a similar manner as Example 1 using a polymer-degrading enzyme. For 10 grams of PET, the theoretical yield of Terephthalic Acid is 8.64 grams. The reactions dosed 10.25 mL of 6M sodium hydroxide after 87.4 hours which corresponds to a reaction completeness of 59.1%. Similar to comparative example 1, the reactor product was stirred and heated to above the melting temperature of the polymer-degrading enzyme for 10 minutes. The stirring was stopped, and the samples were allowed to rest for 30 minutes. Agglomerates of PET settled to the bottom of the vessel resulting in a clear supernatant. The reactor product was filtered on a 8 micrometer filter (Whatman 40, Cytiva) in a buchner funnel using a vacuum pump (IKA MVP10 Basic). The filtration time for this product was timed using a stopwatch and measured to be 6 minutes and 30 seconds. Reactor product prior to heat treatment and filtered product was tested similar to Example 4 for enzyme concentration with both samples being under the limit of detection for the BCA assay. Without wishing to be bound by any particular theory, the polymer-degrading enzyme was, in this example, present in a lower concentration than the enzyme used in Comparative Example 2 and was higher in purity. Accordingly, the amount of biological contaminants that may be present for the BCA assay to detect prior to heat treatment was relatively low.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “at %” is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method, comprising:
in a fluid comprising one or more polypeptides and a plurality of plastic particles, at least partially denaturing a polypeptide in the fluid; and
separating a mixture comprising denatured polypeptide and at least 10% of the plastic particles from the fluid.

2. The method of claim 1, wherein at least one of the polypeptides comprises and/or is derived from a polymer-degrading enzyme or a fragment thereof.

3. The method of claim 1, wherein the fluid has a temperature greater than or equal to the melting temperature of at least one of the one or more polypeptides.

4. The method of claim 1, wherein the plurality of plastic particles have an average maximum dimension less than or equal to 2 millimeters.

5. The method of claim 1, wherein the one or more polypeptides comprise a sacrificial polypeptide.

6. The method of claim 1, wherein the one or more polypeptides comprise a sacrificial polypeptide and a polymer-degrading enzyme.

7. The method of claim 6, wherein the melting temperature of the sacrificial polypeptide is less than the melting temperature of the polymer-degrading enzyme.

8. The method of claim 1, wherein the plurality of plastic particles are suspended in the fluid.

9-13. (canceled)

14. The method of claim 1, wherein the onset of separation occurs is associated with exposing the fluid to a shear rate greater than or equal to 1000 s⁻¹ and less than or equal to 100000 s⁻¹.

15. The method of claim 1, wherein at least 25% of the plastic particles are separated from the fluid.

16-17. (canceled)

18. The method of claim 1, wherein the fluid is at rest for a duration greater than or equal to 10 minutes after the denaturation of polypeptide in the fluid.

19. The method of claim 1, wherein the fluid is at rest when some of the plurality particles associate with some of the polypeptides in the fluid.

20. A method, comprising:
separating a mixture comprising a plurality of plastic particles and one or more denatured polypeptides, from a supernatant comprising a fluid, wherein:
the concentration of at least one of the polypeptides in the supernatant is less than or equal to 1×10⁻³ M.

21. The method of claim 20, wherein the onset of separation occurs is associated with the fluid having a temperature greater than or equal to the melting point of at least one of the one or more polypeptides.

22. The method of claim 20, wherein the separating does not involve filtration and/or centrifugation.

23. The method of claim 20, wherein the concentration of at least one of the polypeptides in the fluid after separation is less than or equal to 1×10⁻³ M and greater than or equal to 1×10⁻⁷ M.

24. A composition, comprising:
one or more denatured polypeptides; and
a plurality of plastic particles, having an average maximum dimension less than or equal to 2 mm, wherein the composition is the product of at least partial isolation from a solution or suspension comprising the plastic particles and the polypeptide.

25. The composition of claim 24, wherein at least one of the polypeptides, prior to denaturation, is a polymer-degrading enzyme.

26. The composition of claim 24, wherein at least some the plurality of plastic particles are bonded to at least some of the polypeptides.

27. The composition of claim 24, wherein at least some of the polypeptides are adsorbed onto at least some of the plurality of plastic particles.

28-38. (canceled)
